(19) United States
(12) Patent Application Publication
(10) Pub. No.: US 2023/0201514 A1
(43) Pub. Date: Jun. 29, 2023

(54) SYSTEMS AND METHODS FOR NITRIC OXIDE GENERATION AND TREATMENT

(71) Applicant: The General Hospital Corporation, Boston, MA (US)

(72) Inventors: Warren M. Zapol, Cambridge, MA (US); Lorenzo Berra, Cambridge, MA (US); Caio Morais, Quincy, MA (US); Stefano Gianni, Boston, MA (US); Ryan W. Carroll, Boston, MA (US); Walfre Franco, Westborough, MA (US); Binglan Yu, Lexington, MA (US); R. Rox Anderson, Boston, MA (US)

(21) Appl. No.: 18/008,316

(22) PCT Filed: Jun. 7, 2021
(86) PCT No.: PCT/US2021/036269

§ 371 Date: Dec. 5, 2022

Related U.S. Application Data
(60) Provisional application No. 63/035,103, filed on Jun. 5, 2020.
(60) Provisional application No. 63/035,451, filed on Jun. 5, 2020.
(60) Provisional application No. 63/035,458, filed on Jun. 5, 2020.
(60) Provisional application No. 63/089,043, filed on Oct. 8, 2020.

(51) Int. Cl. A61M 16/12; A61M 16/10; A61M 16/20

(52) U.S. Cl. CPC A61M 16/12; A61M 16/105; A61M 16/208; A61M 16/1005; A61M 2205/3344; A61M 2202/0266; A61M 2202/0275; A61M 2016/102; A61M 2230/20

(57) ABSTRACT

Systems and methods for generating and delivering nitric oxide are provided. In one aspect, a nitric oxide generator includes an inlet arranged to receive a gas including nitrogen and oxygen, an outlet, a pair of electrodes arranged downstream of the inlet and configured to generate nitric oxide from the gas, a pressure regulator configured to selectively adjust a pressure of the gas surrounding the electrodes, an accumulator in communication with the pressure regulator, a nitric oxide sensor arranged to measure a concentration of nitric oxide at the outlet, and a controller in communication with the pair of electrodes, the pressure regulator, and the nitric oxide sensor. The controller is configured to selectively instruct the pressure regulator to adjust the pressure of the gas surrounding the electrodes in response to the concentration of nitric oxide measured at the outlet by the nitric oxide sensor.

### CROSS-REFERENCE TO RELATED APPLICATIONS

This application claims priority to each of U.S. Provisional Patent Application No. 63/035,103 filed Jun. 5, 2020, U.S. Provisional Patent Application No. 63/035,451 filed Jun. 5, 2020, U.S. Provisional Patent Application No. 63/035,458 filed Jun. 5, 2020, and U.S. Provisional Patent Application No. 63/089,043 filed Oct. 8, 2020, each of which is incorporated herein by reference in its entirety.

### STATEMENT REGARDING FEDERALLY-SPONSORED RESEARCH

This invention was made with government support under, NHLBI B-BIC/NCAI (#U54HL119145), each awarded by the National Institutes of Health. The government has certain rights in the invention.

### BACKGROUND

Nitric oxide (“NO”) can be used to reduce the level of pulmonary artery pressure. For example, NO is approved by the food and drug administration (“FDA”) to treat hypoxic newborns with persistent pulmonary hypertension.

### BRIEF SUMMARY

In some aspects, the present disclosure provides systems and methods for treating sick patients and healthy individuals with inhaled nitric oxide. These systems and methods are applicable for a variety of NO sources, including but not limited to NO from a compressed gas cylinder, electrically generated NO, and chemically derived NO. Also provided is a nitric oxide generator including an inlet arranged to receive a gas including nitrogen and oxygen, an outlet, a pair of electrodes arranged downstream of the inlet and configured to generate nitric oxide from the gas, a pressure regulator configured to selectively adjust a pressure of the gas surrounding the electrodes, an accumulator in communication with the pressure regulator, a nitric oxide sensor arranged to measure a concentration of nitric oxide at the outlet, and a controller in communication with the pair of electrodes, the pressure regulator, and the nitric oxide sensor. The accumulator is configured to add volume to a flow path between the inlet and the outlet to store and maintain the pressure of the gas surrounding the electrodes. The controller is configured to selectively instruct the pressure regulator to adjust the pressure of the gas surrounding the electrodes in response to the concentration of nitric oxide measured at the outlet by the nitric oxide sensor.

In some aspects, the present disclosure provides a nitric oxide generator that includes an inlet arranged to receive a gas including nitrogen and oxygen, an outlet, a pair of electrodes arranged downstream of the inlet and configured to generate nitric oxide from the gas, a nitric oxide sensor arranged to measure a concentration of nitric oxide at the outlet, and a controller in communication with the pair of electrodes and the nitric oxide sensor. The controller is configured to selectively adjust a signal sent to the electrodes to provide a concentration of nitric oxide greater than or equal to about 150 ppm at the outlet.

In some aspects, the present disclosure provides a nitric oxide delivery system that includes an inlet, a first outlet, a main conduit connected between the inlet and the first outlet, a nitric oxide generator including a pair of electrodes configured to generate a predetermined concentration of nitric oxide, and a reservoir connected to the main conduit downstream of the nitric oxide gas source. The reservoir is configured to add a predetermined amount of volume a flow path defined between the inlet and the first outlet. The nitric oxide delivery system further includes a scavenger arranged between the reservoir and the first outlet, a filter arranged between the reservoir and the first outlet, an inspiratory valve arranged downstream of the reservoir and configured to allow gas flow only in a direction from the reservoir toward the first outlet, and an expiratory valve arranged downstream of the first outlet and configured to allow gas flow only in a direction from the first outlet to a second outlet.

In some aspects, the present disclosure provides a nitric oxide delivery system that includes an inlet, a first outlet, a main conduit connected between the inlet and the first outlet, a nitric oxide generator including a pair of electrodes configured to generate a predetermined concentration of nitric oxide, and a reservoir connected to the main conduit downstream of the nitric oxide gas source. The reservoir is configured to add a predetermined amount of volume a flow path defined between the inlet and the first outlet. The nitric oxide delivery system further includes a scavenger arranged between the reservoir and the first outlet, a filter arranged between the reservoir and the first outlet, and a connector arranged downstream of the reservoir and including an inspiratory valve and an expiratory valve. The inspiratory valve is configured to allow flow only in a direction from the reservoir toward the first outlet, and the expiratory valve is configured to allow flow only in a direction from the first outlet to a second outlet. The nitric oxide deliver system further includes an oxygen gas source connected to the connector at a location downstream of the inspiratory valve and configured to provide supplemental oxygen gas to the first outlet.

In some aspects, the present disclosure provides a method for generating and delivering nitric oxide that includes supplying a gas including nitrogen and oxygen to a spark chamber with a pair of electrodes, electrically driving the pair of electrodes to generate a predetermined concentration of nitric oxide, supplying the generated nitric oxide to a reservoir, flowing the generated nitric oxide from the reservoir through a scavenger and an inspiratory valve, mixing supplemental oxygen gas into the generated nitric oxide downstream of the inspiratory valve, and delivering the generated nitric oxide and the supplemental oxygen to an outlet.

In some aspects, the present disclosure provides a method for treating or preventing COVID-19 (SARS-CoV-2) infection. The method includes administering a concentration of nitric oxide gas greater than or equal to 150 ppm to a respiratory system of a patient for at least one inspiration.

In some aspects, the present disclosure provides a method for treating or preventing COVID-19 (SARS-CoV-2) infection in a pregnant patient. The method includes determining an oxygen requirement, upon determining that the oxygen requirement is below a predetermined threshold, administering a concentration of nitric oxide gas greater than or equal to 150 ppm to a respiratory system of a pregnant patient for at least 30 minutes, and upon determining that the oxygen requirement is above the predetermined threshold, continuously administering a concentration of nitric oxide gas greater between about 5 ppm and about 20 ppm to the respiratory system of the pregnant patient and administering a concentration of nitric oxide gas greater than or equal to 150 ppm to the respiratory system of the pregnant patient for at least 30 minutes.

In some aspects, the present disclosure provides a method for generating and delivering nitric oxide. The method includes supplying a gas including nitrogen and oxygen to a spark chamber with a pair of electrodes, electrically driving the pair of electrodes to generate an predetermined inhaled concentration of nitric oxide. The predetermined inhaled concentration of nitric oxide is greater than or equal to about 150 ppm. The method further includes flowing the generated nitric oxide from through a scavenger, delivering the generated nitric oxide to a patient, and monitoring a methemoglobin level of the patient when delivering the generated nitric oxide.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### DESCRIPTION OF THE DRAWINGS

The following drawings are provided to help illustrate various features of non-limiting examples of the disclosure, and are not intended to limit the scope of the disclosure or exclude alternative implementations.

FIG. 1 shows a schematic illustration of a nitric oxide (“NO”) delivery system.

FIG. 2 shows a schematic illustration of an NO generator.

FIG. 3 shows a schematic illustration of another NO generator.

FIG. 4 shows a schematic illustration of another NO generator.

FIG. 5 shows a schematic illustration of an NO delivery sensor system.

FIG. 6 shows a schematic illustration of another NO delivery system.

FIG. 7 shows a flowchart of a process for delivering NO gas to a patient.

FIG. 8 shows an example of a high-pressure electric NO (“eNO”) generator.

FIG. 9 shows a block diagram of a high pressure eNO generator of FIG. 8.

FIG. 10 shows a schematic of the inner components of the high-pressure eNO generator of FIG. 8.

FIG. 11 shows three graphs of NO concentration (ppm) verses air flow rate (L/min) for different duty cycles of the power delivered to an electrical spark generator of the eNO generator of FIG. 8.

FIG. 12 shows a schematic of an experimental setup for testing the eNO generator of FIG. 8.

FIG. 13 shows four graphs of NO concentration (ppm) vs. minute ventilation (L/min) for different pressures within the NO generator, and for different duty cycles of power delivered to the electrical spark generator of the eNO generator of FIG. 8.

FIG. 14 shows a graph of the predicted log SARS-COV-2 value vs. the days after randomization for treatment and control groups after treatment with the eNO generator of FIG. 8.

FIG. 15 shows an example of a portable NO generator.

FIG. 16 shows the parts of the portable NO generator of FIG. 15.

FIG. 17 shows a front isometric view of the portable NO generator of FIG. 15.

FIG. 18 shows a side isometric view of the portable NO generator of FIG. 15, with the cover opened.

FIG. 19 shows an image of a display of a particle counter.

FIG. 20 shows another image of the display of FIG. 19.

FIG. 21 shows a graph of the NO and NO₂ concentration (ppm) versus the time (days) during continuous NO generation with the portable NO generator of FIG. 15.

FIG. 22A shows an isometric view of a new spark plug of the portable NO generator of FIG. 15.

FIG. 22B shows another isometric view of a new spark plug of the portable NO generator of FIG. 15.

FIG. 23A shows an isometric view of the spark plug of FIGS. 22A and 22B after 37 days of use with the portable NO generator of FIG. 15.

FIG. 23B shows another isometric view of the spark plug of FIG. 23A.

FIG. 24 shows a top view of new tubing connections to the NO generation chamber before the 37 days of use with the portable NO generator of FIG. 15.

FIG. 25 shows another top view of the tubing connections to the NO generation chamber after the 37 days of use with the portable NO generator of FIG. 15.

FIG. 26 shows an image of a user breathing high doses of NO generated from air by the portable NO generator of FIG. 15 with a snug fitting mask.

FIG. 27 shows a graph of the NO concentration (ppm) and NO₂ concentration (ppm) of inspired gas delivered to a healthcare worker over time (seconds) with the portable NO generator of FIG. 15.

FIG. 28 shows a graphic representation of a delivery device.

FIG. 29 shows a graph of the representative tracing of NO and NO₂ concentrations over time in minutes during the 160 ppm NO inhalation in a healthy healthcare worker.

FIG. 30 a system for delivering inhaled nitric oxide (NO) during spontaneous breathing.

FIG. 31 shows a schematic illustration of the experimental setup for testing the system of FIG. 30.

FIG. 32 shows a graph of the NO and NO₂ signals synchronized with the airway flow during a bench test with the experimental setup of FIG. 31.

FIG. 33 shows graphs of the changes in inspiration NO (ppm) during incremental inspiratory flow, with and without the reservoir bag using the experimental setup of FIG. 31.

FIG. 34 shows a graph of the inspiratory NO₂ concentrations (ppm) at increasing NO concentrations (ppm) with and without the reservoir bag. FiO₂ was 0.21 in each step with the experimental setup of FIG. 31.

FIG. 35 shows a graph of the nitric oxide concentration (ppm) in static and dynamic conditions (during mechanical ventilation) at different air flows: 5 L/min (black dots), 10 L/min (grey dots) and 15 L/min (white dots) with the experimental setup of FIG. 31.

FIG. 36 shows a graph of NO₂ concentration with increasing NO concentrations (50 ppm, 150 ppm, 200 ppm) at 2 levels of FiO₂ (0.21 and 0.4) with (continuous line) or without (dotted line) the calcium hydroxide scavenger in the experimental setup of FIG. 31.

FIG. 37 shows a graph of NO and NO₂ concentration during 15 minutes of NO gas inhalation using the experimental setup of FIG. 31 at a target NO dose of 160 ppm.

FIG. 38 shows a graph of exhaled nitrogen dioxide concentration (line with circles) during a single breath in a healthy subject, with the solid line representing flow.

FIG. 39 shows an NO delivery device and administration, with panel A of FIG. 39 showing the NO delivery device built from respiratory care components for spontaneously breathing patients, and panel B of FIG. 39 showing NO administration in an adult patient using the delivery device.

FIG. 40 shows graphs of Vital variables and laboratory variables in severe coronavirus disease 2019 patients receiving nitric oxide (NO) treatment, with panel A of FIG. 40 showing cardiopulmonary variables of patients receiving 160 parts per million NO with no negative effect on mean arterial pressure (MAP), panel B of FIG. 40 showing heart rate, panel C of FIG. 40 showing respiratory rate (data shown as median and 95% CI), panel D of FIG. 40 showing that oxygenation was not impaired and showed to be stable for normoxemic and hypoxemic cases, panel E of FIG. 40 showing a course of laboratory results for patient 1 with CRP being C-reactive protein, and panel F of FIG. 40 showing a course of laboratory results for patient 2.

FIG. 41 shows a clinical flowchart for nitric oxide therapy in pregnant patients with coronavirus disease 2019 (COVID-19) at Massachusetts General Hospital.

FIG. 42 shows a schematic illustration of a nitric oxide delivery system for spontaneous breathing patients.

FIG. 43 shows graphs of different parameters for baseline testing, with panel A of FIG. 43 showing saturation of oxygen, panel B of FIG. 43 showing respiratory rate, panel C of FIG. 43 showing mean arterial pressure, and panel D of FIG. 43 showing heart rate before, during and after treatment.

FIG. 44 shows graphs of inflammatory markers before, during, and at discharge, with panel A of FIG. 44 showing the C-Reactive Protein (CRP) over time: one observation for patient, and panel B of FIG. 44 showing the C-Reactive Protein (CRP), Interleukin 6 (IL-6), Lymph count of Patient 6 over time.

FIG. 45 shows an NO delivery system.

FIG. 46 shows graphs of the effect of nitric oxide (NO) inhalation on respiratory rate (RR) and oxygenation over time using the NO delivery system of FIG. 45, with panel A of FIG. 46 showing a graph of SpO₂(%)/FiO₂(%) (n=33 treatments) over time, panel B of FIG. 46 shows the RR (n=75 treatments) over time, and panel C of FIG. 46 shows RR rate over days.

FIG. 47 shows a graph of the methemoglobin concentration during nitric oxide (NO) inhalation therapy (Rx).

FIG. 48 shows a timeline of antibiotic therapies, antibiotic susceptibility pattern of the primary pathogen (Burkholderia cepacia complex (Burkholderia multivorans)), and colony forming units (CFU) of this pathogen relative to the initiation of high-dose inhaled nitric oxide therapy on day 1.

FIG. 49 shows a graph of the relationship between therapies, vital signs, and inflammatory markers in an adolescent with CF, treated with intravenous antibiotics and high-dose inhaled nitric oxide, with the first and second hospital admissions being shown on the left and right panels of FIG. 49, respectively, and duration of therapy with either intravenous antibiotics or inhaled nitric oxide is shown at the top of FIG. 49.

FIG. 50 shows a schematic representation of the inhaled nitric oxide delivery system.

FIG. 51 shows a graph of the methemoglobin levels (mean±standard deviation) from data recorded at 5-minute intervals during 30-minute and 60-minute inhalations of high-dose nitric oxide with the delivery system of FIG. 50.

FIG. 52 shows a flowchart of a proposed study.

FIG. 53 shows a nitric oxide gas delivery system.

FIG. 54 shows an apparatus used to deliver inhaled nitric oxide.

FIG. 55 shows a graph of a non-invasive peripheral saturation of methemoglobin (SpMet) monitoring before NO gas start and at the end of NO administration, with iNO: pressurized NO/N₂ cylinder; eNO: electric NO generator.

FIG. 56 shows a graphs of Nitric oxide (NO) and nitrogen dioxide (NO₂) concentration during of the 15-minute study gas administrations, with panel A of FIG. 56 depicting the use of a pressurized cylinder (iNO) as a nitric oxide source, and panel B of FIG. 56 depicting the use of gas source being the electric NO generator (eNO).

FIG. 57 shows a graphs of NO and NO₂ concentrations for the iNO and eNO, with panel A of FIG. 57 showing the NO concentration for the iNO and eNO with the intra-tidal concentration variation shown (minimum on the left and maximum on the right for each NO source), and panel B of FIG. 57 showing the NO₂ concentration for the iNO and eNO with the intra-tidal concentration variation shown (minimum on the left and maximum on the right for each NO source).

FIG. 58 shows various graphs of inspiratory and expiratory NO (thicker line in the upper row) and nitrogen dioxide (NO₂) (line in the bottom row with circles) concentrations during three consecutive breaths, which is panel A (the first column), panel B (the middle column), and panel C (the right column).

FIG. 59 shows graphs of peripheral oxygen saturation (SpO₂) in panel A and heart rate (HR) in panel B before NO gas administration and at the end of NO administration, with iNO: pressurized NO/N₂ cylinder and eNO: electric NO generator.

### DETAILED DESCRIPTION

The use of the terms “downstream” and “upstream” herein are terms that indicate direction relative to the flow of a fluid and/or gas. The term “downstream” corresponds to the direction of flow, while the term “upstream” refers to the direction opposite or against the direction of flow.

In general, NO delivery is underutilized in practice, which can be tied to its difficulty in obtaining NO gas sources (e.g., pressurized NO/N₂ gas cylinders). In addition, NO has been largely clinically absent for treating other conditions aside from individuals with persistent pulmonary hypertension. NO gas is typically generated and pressurized in gas cylinders with other generally non-reactive gases (e.g., N₂), which are subsequently delivered to medical facilities (e.g., hospitals). Because NO reacts spontaneously to form other undesirable byproducts including nitrogen dioxide (NO₂) in the gas cylinder, and even more so upon interaction with ambient air (e.g., that has a significant level of O₂ gas), proper NO delivery for patients has been difficult. Thus, with difficulty in delivering NO gas to patients, few manufacturers of NO gas remain at least due to the diminished demand, which makes NO gas sources scarce.

Even with NO gas sourced, NO delivery for patients can still be difficult. For example, the large cylinders of NO gas make portability of NO gas within the medical facility limited (e.g., due to the inability to easily transport the NO gas cylinders). Thus, in some cases, only specific rooms have the NO gas cylinders available for NO delivery, which can lead to logistical issues (e.g., patients being in NO stocked rooms without requiring NO gas treatment). NO gas in cylinders is packaged with a balance of nitrogen. Thus, delivery of high concentrations of NO from a cylinder can significantly decrease the amount of oxygen delivered to the patient (e.g. 10% oxygen reduction for 80 ppm inhaled concentration from a 800 ppm NO cylinder).

Some aspects of the disclosure provide advantages to these issues (and others) by providing improved systems and methods for NO generation, delivery, treatment and/or prevention. For example, some embodiments of the disclosure contemplate the usage of relatively high inhaled concentrations of NO gas (e.g., greater than about 150 ppm) for treating and preventing respiratory illnesses (e.g., COVID-19 induced pneumonia, bacterial induced pneumonia, etc.) that negatively impact normal gas exchange in the respiratory system. For example, the systems and methods described herein provide high levels of NO gas concentrations in clinically relevant flow rates without significant concomitant reductions in inhaled oxygen levels. High concentration NO can greatly treat and prevent respiratory diseases—especially those caused by pathogens—because high levels of NO gas concentrations can improve the negatively impacted gas exchange in the lungs, can reduce inflammation (e.g., decrease an overly active in a particularly non-helpful manner), and can target and deactivate (e.g., “kill”) pathogens (e.g., viruses, bacteria, fungi including molds) that cause the respiratory illness. For example, while NO gas cylinders in theory could deliver high NO gas concentrations at high flow rates, current systems do not exist that appropriately deliver high NO gas concentrations in a safe manner (e.g., without chemical byproducts, such as NO₂) and either significant impacts on oxygen levels or requiring supplementary oxygen.

Some aspects of the disclosure provide advantages to these issues (and others) by providing a portable NO generator that can generate high NO gas concentrations in clinically relevant flow rates (2-150 lpm). The portable NO generator can include all the necessary components within the same housing, including an electrical spark generator, a power source (e.g., a rechargeable battery), pumps, filters, scavengers, etc. In this way, the portable NO generator can function as a standalone unit (e.g., a self-contained system), which prevents the usage of NO gas cylinders that are cumbersome, not easily portable, and require operator skill to appropriately utilize. In some cases, the portable NO generator can include multiple electrical spark generators, or gliding arc electrodes to further increase the NO generation ability of the NO generator. In addition, the scavengers, filters, and the electrical spark generators can be removably coupled to the housing, which can make replacing these components easier, including when the component has reached its maximum number of uses (e.g., and needs to be replaced). This is particularly helpful because the NO generator creates higher production levels (the mathematical product of NO concentration with mass flow rate) of NO gas, which can cause components (e.g., the electrical spark generator, the filter, the scavenger, etc.) to reach their end of lifetime more quickly than conventional NO generators.

Some aspects of the disclosure also provide an NO delivery system that is configured to deliver relatively high inhaled concentrations of NO gas (e.g., greater than about 150 ppm). The NO delivery system can deliver the high concentration of NO gas by using inspiration and expiration driven by the patient's diaphragm or via mechanical ventilation with a ventilator. In addition, the NO delivery system can deliver high production levels of NO gas, while still maintaining the delivery of normal (or high) fraction of inspired oxygen levels (e.g., where normal fraction of inspired oxygen levels are about 0.21, and a high fraction of inspired oxygen is greater than about 0.21 including up to about 0.42, and sometimes as high as 100%). This can be particularly helpful because the relatively high NO gas level and the normal (or high) O₂ level can work together in tandem to greatly improve oxygen delivery to the patient (e.g., in a mutualistic manner, where the high NO concentration opens the blood vessels and the higher O₂ level increases the propensity for oxygen exchange in the lungs).

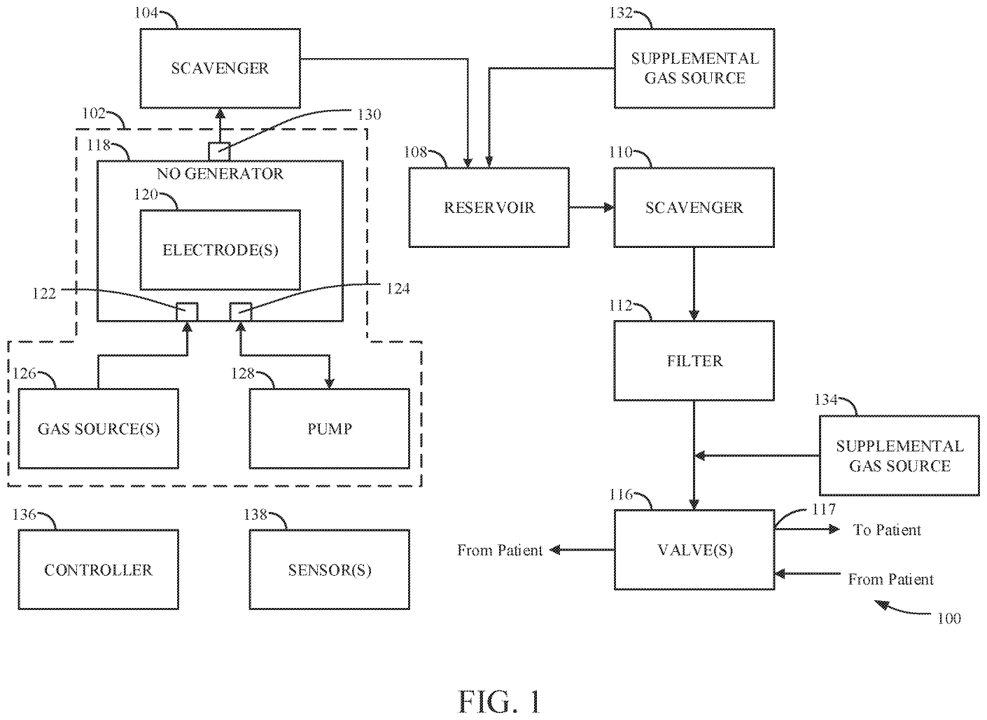

FIG. 1 shows a schematic illustration of a nitric oxide (“NO”) delivery system 100. The NO delivery system 100 can include an NO gas source 102, a first scavenger 104, a reservoir 108, a second scavenger 110, a filter 112, and valve(s) 116. The NO gas source is configured to provide NO gas to the first scavenger 104 and other portions of the NO delivery system 100. In some cases, the NO gas source 102 can be a tank filled with NO gas (and other gases such as N₂). For example, the NO gas source 102 can include a pressurized cylinder of NO gas that when connected to the first scavenger 104 (or other components of the NO delivery system 100), delivers NO gas to the first scavenger 104. As another example, the NO gas source 102 can include a container filled with NO gas and a pump in communication with the container to drive NO gas out of the container and to the first scavenger 104 (or other portions of the NO delivery system).

In some aspects, and as illustrated in FIG. 1, the NO gas source 102 can include an NO generator 118 that is configured to generate NO, such as from a pulsed electric arc reaction between gaseous oxygen and nitrogen. The NO generator 118 can include a housing that is sealed from the ambient environment to define an interior volume. In some cases, the NO generator 118 can include an electrode, at least one pair of electrodes, or a gliding arc 120, which can be positioned within the interior volume of the housing of the NO generator 118. The NO generator 118 can include ports 122, 124 that are each in fluid communication with the interior volume of the housing. The port 122 can function as an inlet that receives gas from a gas source 126 (e.g., oxygen gas, ambient air, or a mixture of oxygen and nitrogen) and directs the gas into the interior volume of the housing. In some cases, the gas source 126 can include a pump to drive the gas into the interior volume of the NO gas source 102. In some configurations, while the NO generator 118 is illustrated in FIG. 1 as having a single port 122 (e.g., a single inlet), in other configurations, the NO generator 118 can have multiple ports 122. In this case, for example, a second gas source (e.g., different from the gas source 126) can be in in fluid communication with the other port 122. In this way, the gas source 126 can be an ambient air source (e.g., or a nitrogen gas source), and the second gas source can be an oxygen gas source that can also include a pump to drive the oxygen gas into the other port 122. Thus, the gas source 126 and the oxygen gas source can provide a substantially (e.g., deviating by less than 20%) one to one ratio of nitrogen gas to oxygen gas into the interior volume of the housing of the generator 118 (e.g., which can facilitate the generation of higher amounts and concentrations of NO).

In some aspects, the NO generator 118 can include a pump 128 that can be coupled to and in fluid communication with the port 124. The pump 128 can be in fluid communication with the interior volume of the housing of the NO generator 118. The pump 128, when active, can drive gas out of the interior volume of the housing of the NO generator 118. In this case, for example, a first one-way valve can be in communication with the port 122 and a second one-way valve can be in communication with a port 130 that is also in communication with the interior volume. In this way, with the one-way valve, gas flow is blocked from flowing out of the interior volume and through the port 122, and gas is blocked from flowing into of the interior volume and through the port 130. When gas is evacuated out of the interior volume of the housing, via the pump 128, gas does not backflow to the gas source 126 (and other gas sources), and NO (and other gases) downstream of the port 130 are not forced back into the interior volume of the housing. In some cases, the pump 128 can be in gas communication with the ambient environment (or a container) to direct the evacuated gas out into the ambient environment (or into a container, such as for disposal).

In some aspects, the electrodes 120 can be positioned within the interior volume of the housing of the NO generator 118. As power is directed to the electrodes 120, a spark (or in some cases an electrical arc) is produced between the electrodes 120 thereby generating NO within the interior volume (e.g., when gaseous oxygen and gaseous nitrogen are present). In some aspects, one electrode of the pair of electrodes 120 can be connected to a power source (not shown), while the other electrode can be connected to ground (e.g., electrical ground). However, in alternative configurations, the one electrode can be electrically connected to a positive terminal of a power source, while the other electrode can be electrically connected to a negative terminal of the power source. In some cases, the pair of electrodes 120 can be structured as a sparkplug (e.g., with a central and lateral electrode). In this case, for example, the spark plug can be a single electrode sparkplug (e.g., having a central electrode and a single lateral electrode), or a multi electrode sparkplug, such as a dual electrode sparkplug (e.g., having a central electrode and two lateral electrodes), a triple electrode sparkplug, etc.

In some aspects, the NO generator 118 can include multiple pairs of electrodes 120. In this case, for example, the multiple pairs of electrodes 120 can be situated within the interior volume of the housing of the NO generator 118. In some cases, and as described below, each pair of electrodes 120 can be situated within its own housing (i.e., an electrode housing), isolated from (but in communication with) the interior volume of the housing of the NO generator 118. In this case, each pair of electrodes 120 can be removably coupled to the respective electrode housing, and each electrode housing can be removably coupled to the housing of the NO generator 118. In this way, each electrode housing (or alternatively each pair of electrodes) can be easily replaced. In some cases, each electrode housing, when coupled to the housing of the NO generator 118 can be electrically connected to the power source (e.g., and electrically disconnected when decoupled from the housing). In addition, each electrode pair when coupled to the respective electrode housing can be electrically connected to the power source. In this way, each electrode housing and each electrode pair or a gliding arc can be easily decoupled as needed (e.g., for replacement, or such as when less or more electrode pairs are needed based on the desired NO concentration). In some aspects, each pair of electrodes can have an electrical switch that, when activated, allows power from the power source to flow to the respective pair of electrodes. In this way, a controller (not shown) of the NO gas source 102 can recruit the desired number of electrode pairs, which can be based on the desired concentration of NO to be generated. For example, at relatively higher NO concentrations, additional pairs of electrodes can be recruited to generate NO.

As shown in FIG. 1, NO generated within the interior volume of the housing of the NO gas source 102 (and other gases) are emitted though the port 130 and to the first scavenger 104 situated downstream of the port 130. Alternatively, such as when the NO gas source 102 is implemented as a (pressurized) tank of NO gas, the tank can be in fluid communication with and positioned upstream of the first scavenger 104. Regardless of the configuration, the first scavenger 104 is configured to remove undesirable gaseous byproducts or other chemical contaminants from the gas delivered by the NO gas source 102. For example, the first scavenger 104 can remove nitrogen dioxide (NO₂), ozone (O³), and other undesirable chemical contaminants from the output gas. As a more specific example, the first scavenger 104 can be an oxide, a hydroxide (e.g., calcium hydroxide), etc., that can be crushed, powdered, formed into beads, etc. In this case, for example, the first scavenger 104 can be positioned and secured within a scavenger housing. For example, the scavenger housing can have an inlet and an outlet with the first scavenger 104 positioned between the inlet and the outlet. In some cases, the first scavenger 104 can have a monolith substrate that defines a three-dimensional (“3D”) shape (e.g., a disc, a block, a cylinder, a cube, a prism, etc.) that can be porous (e.g., defining a plurality of pores). In some cases, the monolith substrate can have a plurality channels directed though the 3D shape (substantially) larger than the pores, and thus the monolith substrate can have a honeycomb structure (e.g., with channels of various shapes, such as square, prisms, etc.). In some cases, this monolith substrate can be a catalytic converter, such as a selective catalytic reduction converter, which can be formed out of various materials (e.g., ceramics, such as oxides including titanium oxide, aluminum oxide, etc.) and lined with catalysts (e.g., oxides of base metals or other metals, hydroxides of metals including calcium hydroxide, magnesium oxide, etc.). In some configurations, the selective catalytic converter can be more selective for NO₂ over NO to remove away the NO₂, while preventing the decomposition of NO (e.g., the reduction of NO). For example, the catalysts can be relatively weak catalysts (e.g., metal hydroxides rather than metal oxides) that reduce NO₂ while preventing large amounts of NO from being reduced. In some aspects, the first scavenger 104 in addition to removing NO₂ from the gas, can also remove O₃ and carbon monoxide (CO) from the output gas. For example, the first scavenger 104 can reduce O₃ into gaseous oxygen, and the first scavenger 104 can reduce CO into CO₂.

In some aspects, while the first scavenger 104 is illustrated in FIG. 1 as being downstream of the NO generator 118 including the port 130 (e.g., that functions as an outlet), the first scavenger 104 can be positioned within the port 130, or can be positioned within the interior volume of the housing of the NO generator 118. In this way, gas within the NO generator 118 must pass though the first scavenger 104 before being directed to the reservoir 108.

After the gas from the NO gas source passes through the first scavenger 104, the gas is directed to the reservoir 108, which can be in fluid communication with the first scavenger 104. The reservoir 108 can also receive gas from a supplemental gas source 132 (e.g., a medical air gas source, or supplemental O₂ gas source). Similarly to the gas source 126, the supplemental gas source 132 can be implemented in different ways, such as being pressurized gas within a tank, or a container that includes a pump to drive gas out of the container and into the reservoir 108. Regardless of the configuration, gas from the supplemental gas source 132 that is introduced into the reservoir 108 and gas emitted from the first scavenger 104 (and the NO gas source 102) mixes and flows into the reservoir 108. In this way, and as described below, the reservoir 108 fills with NO gas diluted with gas from the supplemental gas source 132. In some cases, the supplemental gas source 132 may be omitted and the gas output from the NO generator 118 may flow directly into the reservoir 108.

The reservoir 108 is situated downstream of the NO gas source 102, the first scavenger 104, and the valve 106, and fills with NO gas diluted with gas from the supplemental gas source 132. The reservoir 108 can add a predetermined amount of volume the flow path between the NO generator 118 and an outlet 117 in fluid communication with a patient. In this way, the reservoir 108 can contain and maintain a substantially constant concentration of NO so that NO gas delivery to the patient is regulated (e.g., does not fluctuate beyond a predetermined tolerance of a particular concentration). For example, the added volume provided by the reservoir 108 may average the NO concentration to decrease concentration variability. In some aspects, the predetermined tolerance may be less than about plus or minus 5%, less than about plus or minus 10%, or less than about plus or minus 20%.

In some cases, the reservoir 108 can be expandable as a chamber. For example, the reservoir 108 can be formed out of an expandable material (e.g., polymers such as rubber, etc.) that can, when filled with gas, expand to store the pressurized gas. In some cases, the reservoir 108 can be formed out of different materials (e.g., non-latex materials, due to latex allergies). In some aspects, the reservoir 108 can define a volume of greater than about 0.25 liters (L), greater than about 0.5 L, greater than about 1 L, greater than about 2 L, or about 3 L. In general, the larger volume defined by the reservoir 108 can be correlated with an increased ability of the reservoir to maintain and stabilize the concentration of NO at the outlet 117. The particular size chosen for the reservoir 108 may be governed by the particular flow and NO concentration requirements for a given application.

In some aspects, the second scavenger 110, which can be structured in a similar manner as the first scavenger 104, can be positioned downstream of the reservoir 108. In this way, as NO gas that is located within the reservoir 108 flows downstream, the NO gas passes through the second scavenger 110 to remove any additional chemical contaminants from NO gas. For example, in some cases, the NO gas can come in contact with gaseous oxygen within the reservoir 108 and form NO₂, which can be removed (e.g., reduced) from the NO gas after passing through the second scavenger 110. In some cases, such as when the NO gas source 102 includes the NO generator 118, the size (e.g., the volume) of the second scavenger 110 can be smaller than the size (e.g., the volume) of the first scavenger 104. In this way, the second scavenger 110 can be made smaller because there are less chemical contaminants than at the site of NO generation (e.g., electrical sparks or arcs generate higher amounts of NO₂ and other chemical byproducts than does the spontaneous formation of them when the precursors are housed in the same container). In some configurations, the NO delivery system 100 may include a single scavenger (e.g., either the first scavenger 104 or the second scavenger 110).

In some aspects, the filter 112 can be in fluid communication with the second scavenger 110 and can be positioned downstream of the reservoir 108. In some configurations, the filter 112 can be positioned upstream of the reservoir 108. The filter 112 can remove particulates, including pathogens (e.g., viruses, bacteria, fungi, etc.), and can block passage of aerosolized materials (e.g., generated from persons, which can be pathogen laden or generated by erosion of the electrodes 120). In some cases, the filter 112 can include a filter housing that houses the filter 112, which can include an input to receive gas from the reservoir 108, and an output to deliver gas to the valve 114. In this case, the filter 112 can be positioned between the input and output of the filter housing. The filter 112 can include pores sized to block particles of different sizes from flowing downstream though the filter 112. For example, the filter 112 can have pores sized to block at least 99.95% of particles with sizes less than or equal to 0.3 μm. Accordingly, in some cases, the filter 112 can be a high-efficiency particulate air (“HEPA”) filter.

In some aspects, the gas flowing from the filter 112 can be mixed with a gas (e.g., oxygen) from a supplemental gas source 134 (e.g., an oxygen gas source), which can be implemented in similar ways as the previously described gas sources (e.g., being pressurized tanks of gas, including a container with a pump to drive the gas out of the container, etc.). In some cases, such as when the gas is oxygen (e.g., from the supplemental gas source 134), introducing the oxygen gas close to the patient (e.g., downstream of the filter 112) allows less time for the oxygen to react with the NO to form undesirable NO₂. In other words, this allows for simultaneous oxygen gas and NO gas delivery to a patient, in which oxygen gas delivery can be particularly important for patients that have pneumonia or COVID-19, or other conditions in which gas exchange in the lungs has been worsened.

In some aspects, the NO delivery system 100 can include one or more valves 116 that are positioned downstream of the filter 112. The valve 116 is configured to allow gas flow from the reservoir 108 (e.g., including NO gas therein) during inspiration of the patient, while blocking gas flow to the reservoir 108 during expiration of the patient (e.g., in other words blocking back flow in an upstream direction to the reservoir 108) and allowing this gas to flow into the ambient environment. This can be implemented in different ways. For example, in some cases, the valve 116 can be a three-way valve having at least two positions. During inspiration, a negative pressure is generated (e.g., by the inhalation of the patient), which causes the valve 116 to be in a first position, allowing gas communication between the reservoir 108 (e.g., including the NO gas) and a first outlet of the valve 116, while blocking fluid communication between a second outlet (in fluid communication with the ambient environment) and either of the input or the first output of the valve 116. Conversely, during expiration, a positive pressure is generated (e.g., by expiration of the patient), which causes the valve 116 to be in a second position, allowing fluid communication between the first output of the valve 116 and the second output of the valve 116 (including the ambient environment), while blocking fluid communication between the input of the valve 116 and either of the outputs of the valve 116. In this way, NO gas flows to the respiratory system of the patient during inhalation (e.g., via the first output), triggered by the electronics of the ventilator but does not escape into the ambient environment during expiration (e.g., via the second output). In some cases, the three way valve can have a spring loaded valve seat that can switch between the first and second positions.

In other configurations, rather than the valve 116 being a three-way valve, the NO delivery system 100 can have two one-way valves 116. In this case, for example, the NO delivery system 100 can have a three port connector, with two ports each having a one-way valve 116. The first one way valve can be oriented in a first port of the three port connector so that gas flow is blocked from flowing from a third port (from the patient) of the three port connector and upstream through the first port, while gas flow is allowed to flow downstream through the first port and through the third port. The second one way valve can be oriented in second port so that gas flow is blocked from flowing from the ambient environment and through the third port (and into the third port), while gas flow is allowed to flow from the third port and out through the second port into the ambient environment. Regardless of the configuration, the valve 116 (or valves 116) can allow NO rich gas to flow to the patient during inspiration, while allowing CO₂ rich gas to flow out into the ambient environment (and not upstream) during expiration.

In some aspects, the NO delivery system 100 can include a controller 136 and sensors 138. The controller 136 can be in communication with some (or all) of the components within the NO delivery system 100, as appropriate. For example, the controller 136 can send instructions to components of the NO delivery system 100 (e.g., to cause a component to implement a task), and the controller 136 can receive data from components of the NO delivery system 100. In some configurations, the controller 136 can be in electrical communication (e.g., wired or wireless) with the gas source 126, the pump 128, and the electrodes 120. The controller 136 can be implemented in different ways. For example, the controller 136 can include typical components used such as a processor, memory, a display, inputs (e.g., a keyboard, a mouse, a graphical user interface, a touch-screen display, etc.), communication devices, etc. In some cases, the controller 136 can simply be implemented as a controller having a processor and memory. The controller 136 can communicate with other controllers and systems. The controller 136 can receive and share information from a mechanical ventilator.

In some aspects, the NO delivery system 100 can include one or more sensors 138 that can be in electrical communication (e.g., wired or wireless) with the controller 136 (or other controller, such as a controller device of the NO generator 118). For example, the one or more sensors 138 can include a flow sensor, a pressure sensor, a temperature sensor, a NO sensor, a NO₂ sensor, an O₂ sensor, etc., positioned at various locations along the NO delivery system 100. For example, the NO delivery system 100 can include an NO sensor, and an NO₂ sensor downstream of the filter 111 (e.g., or downstream of the output 117). The NO sensor can sense the concentration of NO (e.g., the amount of NO) in the inhalation gas stream (e.g., in parts per million (“ppm”)), while the NO₂ sensor can sense the concentration of NO₂ (e.g., the amount of NO₂) in the inhalation gas stream. In this way, the actual levels of NO and NO₂ that the patient is receiving is closer to the measured levels of NO and NO₂ (e.g., rather than when the NO sensor and NO₂ sensors are positioned more upstream). In some cases, the NO generator 118 can also include an NO sensor and an NO₂ sensor positioned within the interior volume of the housing of the NO generator 118, or positioned upstream of the NO generator 118 (and downstream of the reservoir 108). In this way, the controller 136 can ensure that the reservoir 108 holds a substantially constant concentration of NO (e.g., a predetermined concentration of NO). For example, the NO delivery system 100 can include a flow sensor (and a pressure sensor) in fluid communication with the port 130. Then, the controller 136 can change the flow rate of gas delivered by the supplemental gas source 132 (e.g., by changing a valve position, or by changing the speed of the pump of the supplemental gas source 132), based on the sensed NO concentration (the sensed flow rate, and the sensed pressure). In this way, the concentration level of NO gas upstream of the reservoir 108 can be adjusted to the predetermined concentration of NO prior to reaching the reservoir 108. This allows the reservoir 108 to contain a substantially stable (and predetermined) concentration of NO gas regardless of amount produced by the NO generator 118. In other words, the controller 136 can compensate for fluctuations in NO generation by the NO generator 118.

In some aspects, the NO generator 118 can include an N₂ sensor, an O₂ sensor, and an NO₂ sensor positioned within the interior volume of the housing of the NO generator 118. For example, the N₂ sensor can sense the concentration of N₂ gas within the interior volume, the O₂ sensor can sense the concentration of O₂ gas within the interior volume, and the NO₂ sensor can sense the concentration of NO₂ gas within the interior volume. Each of these parameters (e.g., the concentration of N₂ gas, O₂ gas, and NO₂ gas within the interior volume) can be used by the controller 136 to control the gas source 126 and the pump 128 to better regulate gas contents within the interior volume to facilitate higher generation of NO and lower generation of byproducts (e.g., NO₂). For example, the controller 136 can cause the gas source(s) 126 to change the flow of the gas delivered to the interior volume of the housing (e.g., by changing a position of a valve, by changing the speed of the pump, etc.), based on the sensor value from the N₂ sensor, or the sensor value from the O₂ sensor. In this way, such as when the O₂ concentration falls below a threshold value (or the N₂ concentration exceeds a threshold value), the controller 136 can cause the gas source(s) 126 to decrease or increase gas flow (e.g., by adjusting the speed of the pump, adjusting the opening of the valve) into the interior volume of the housing to balance the ratio between the N₂ and O₂ until it reaches a predetermined ratio (e.g., a one to one ratio), which can be facilitate a higher level of NO formation for a given flow rate.

In some aspects, as an alternative to or in addition to the pressure, flow, and sensor-based control described herein, the controller 136 can actively control the electrodes 120 based on a commanded NO concentration, a maximum predetermined NO₂ concentration, or both. For example, the controller 136 may be configured to send an electrical signal (e.g., a pulsed square wave) to the electrodes 120 to initiate a series of sparks and the generation of NO. The electrical signal may be selectively modified by the controller 136 (e.g., by increasing/decreasing duty cycle, pulse amplitude/width, and/or a pulse period and/or a frequency) to modify an NO concentration being output by the NO generator 118. In some aspects, the controller 136 may be configured to selectively adjust a signal sent to the electrodes to provide an inhaled concentration of nitric oxide greater than or equal to about 150 ppm at the outlet 117. In some aspects, the controller 136 may be configured to selectively adjust a signal sent to the electrodes 120 to provide a concentration of nitric oxide at the outlet 117 greater than or equal to about 200 ppm, or greater than or equal to about 300 ppm, or greater than or equal to about 400 ppm, or greater than or equal to about 500 ppm, or greater than or equal to about 600 ppm, or greater than or equal to about 700 ppm, or greater than or equal to about 800 ppm, or greater than or equal to about 900 ppm, or greater than or equal to about 1000 ppm, or greater than or equal to about 1100 ppm, or greater than or equal to about 1200 ppm, or greater than or equal to about 1300 ppm, or greater than or equal to about 1400 ppm, or greater than or equal to about 1500 ppm, or greater than or equal to about 1600 ppm, or greater than or equal to about 1700 ppm, or greater than or equal to about 1800 ppm, or greater than or equal to about 1900 ppm, or greater than or equal to about 2000 ppm, or greater than or equal to about 2100 ppm, or greater than or equal to about 2200 ppm, or greater than or equal to about 2300 ppm, or greater than or equal to about 2400 ppm, or greater than or equal to about 2500 ppm. In some aspects, the controller 136 may be configured to selectively adjust a signal sent to the electrodes 120 to provide a concentration of nitric oxide in the NO generator 118 (e.g., within a spark chamber) greater than or equal to about 200 ppm, or greater than or equal to about 300 ppm, or greater than or equal to about 400 ppm, or greater than or equal to about 500 ppm, or greater than or equal to about 600 ppm, or greater than or equal to about 700 ppm, or greater than or equal to about 800 ppm, or greater than or equal to about 900 ppm, or greater than or equal to about 1000 ppm, or greater than or equal to about 1100 ppm, or greater than or equal to about 1200 ppm, or greater than or equal to about 1300 ppm, or greater than or equal to about 1400 ppm, or greater than or equal to about 1500 ppm, or greater than or equal to about 1600 ppm, or greater than or equal to about 1700 ppm, or greater than or equal to about 1800 ppm, or greater than or equal to about 1900 ppm, or greater than or equal to about 2000 ppm, or greater than or equal to about 2100 ppm, or greater than or equal to about 2200 ppm, or greater than or equal to about 2300 ppm, or greater than or equal to about 2400 ppm, or greater than or equal to about 2500 ppm. In some aspects, the controller 136 may be configured to selectively adjust a signal sent to the electrodes 120 to provide a concentration of nitric oxide at the outlet 117 between about 150 ppm and about 2500 ppm, or between about 300 ppm and about 2500 ppm, or between about 500 ppm and about 2500 ppm, or between about 1000 ppm and about 2500 ppm, or between about 150 ppm and about 2000 ppm, or between about 300 ppm and about 2000 ppm, or between about 150 ppm and about 2500 ppm, or between about 500 ppm and about 2000 ppm, or between about 1000 ppm and about 2000 ppm. In some aspects, the controller 136 may be configured to selectively adjust a signal sent to the electrodes 120 to provide a concentration of nitric oxide in the NO generator 118 (e.g., within a spark chamber) between about 150 ppm and about 2500 ppm, or between about 300 ppm and about 2500 ppm, or between about 500 ppm and about 2500 ppm, or between about 1000 ppm and about 2500 ppm, or between about 150 ppm and about 2000 ppm, or between about 300 ppm and about 2000 ppm, or between about 150 ppm and about 2500 ppm, or between about 500 ppm and about 2000 ppm, or between about 1000 ppm and about 2000 ppm.

In some aspects, the one or more sensors 138 can include physiological sensors used to monitor physiological characteristics of the patient (e.g., during NO delivery). For example, the physiological sensors can include a blood oxygenation sensor, a methemoglobin sensor, an blood oxygenation sensor, a blood pressure sensor, a heart rate sensor, an ECG, etc., each of which can be used to control or monitor delivery of NO to the patient. As a more specific example, the methemoglobin sensor can sense the methemoglobin level of the patient, while the blood oxygenation sensor can sense the blood oxygenation level of the patient. The controller 136 can then augment or monitor delivery of NO to the patient based on the methemoglobin level or the blood oxygenation level exceeding a threshold. In particular, the controller 136 can increase the flow of gas from the gas source 126 or the supplemental gas sources 132, 134 thereby diluting the concentration of NO, can stop the sparking of the electrodes 120, and/or can activate a stop valve (not shown) that blocks flow of NO gas through the NO delivery system 100 (e.g., blocks flow from the NO source 102 and to the reservoir 108, or blocks flow from the reservoir 108 and to a downstream component), when the methemoglobin level is above a predetermined threshold, and/or a blood oxygenation level is below a predetermined threshold. In some aspects, the one or more sensors 138 can include a pulse co-oximeter.

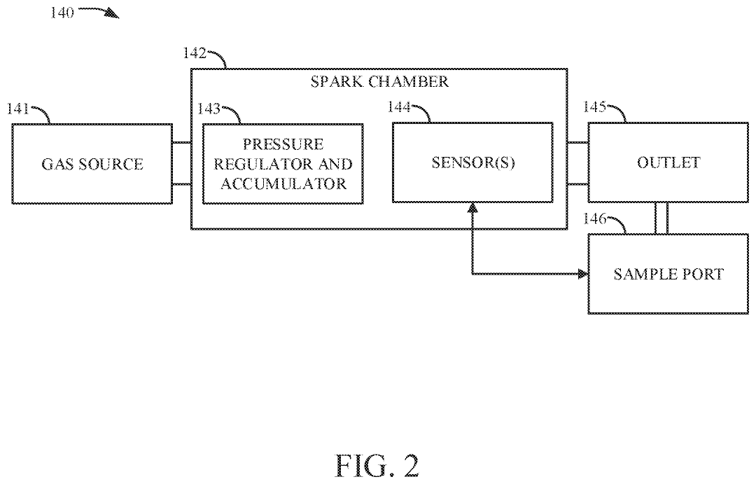

FIG. 2 shows a schematic illustration of an NO generator 140, which can be a specific implementation of the NO generator 118. The NO generator 140 can include a gas source 141, a spark chamber 142, a pressure regulator 143, sensors(s) 144, an outlet 145, and a sample port 146. The spark chamber 142 may include one or more pairs of electrodes configured to produce NO within the spark chamber 142 (e.g., when gaseous oxygen and gaseous nitrogen are present).

The pressure regulator 143 is positioned downstream of the gas source 141, which can be an ambient air source, and is configured to stabilize or adjust the pressure delivered within the spark chamber 142. That is, the pressure regulator 143 may be configured to selectively adjust a pressure surrounding the electrodes. In some cases, the NO generator 140 can also include an accumulator in fluid communication with the pressure regulator 143 (e.g., positioned upstream or downstream of the pressure regulator 143). The accumulator receives gas from the gas source 141, and can store the pressure of gas so that the spark chamber 142 receives gas at consistently the pressure set by the pressure regulator 143. In other words, the accumulator adds volume to the flow path between the inlet (e.g., the gas source 141) and the outlet 145 to store and maintain the pressure of gas delivered by the gas source 141. In some cases, the pressure regulator 143 can be adjusted to change the output pressure of the pressure regulator 143. For example, the pressure regulator 143 can be an electronic or manual pressure regulator, which can be selectively adjusted to vary its output pressure (e.g., via the instruction by a controller or manual manipulation by a user) that is delivered to the spark chamber 142. While the pressure regulator and accumulator 143 are illustrated as being positioned inside the spark chamber 142, in other configurations, the pressure regulator and accumulator 143 can be positioned outside of the spark chamber 142.

The NO generator 140 can include the sensors 144, which can include a pressure sensor, a flow sensor, a NO sensor, a NO₂ sensor, etc. In some aspects, the sensors 144 can sense various related parameters and allow for control of pressure within the spark chamber 142, including adjusting the speed of a pump (e.g., to increase or decrease the pressure delivered to the spark chamber 142), adjusting the output pressure of the pressure regulator 143 (e.g., to increase or decrease the pressure within the spark chamber 142), etc.

The NO sensor can sense the concentration of NO (e.g., the amount of NO) at the sample port 146 (e.g., in parts per million (“ppm”)), while the NO₂ sensor can sense the concentration of NO₂ (e.g., the amount of NO₂) at the sample port 146. In some cases, the NO generator 140 can also include an NO sensor and an NO₂ sensor positioned externally from the spark chamber 142. In addition, the flow rate of gas out of the NO outlet 145 can be sensed by a flow sensor.

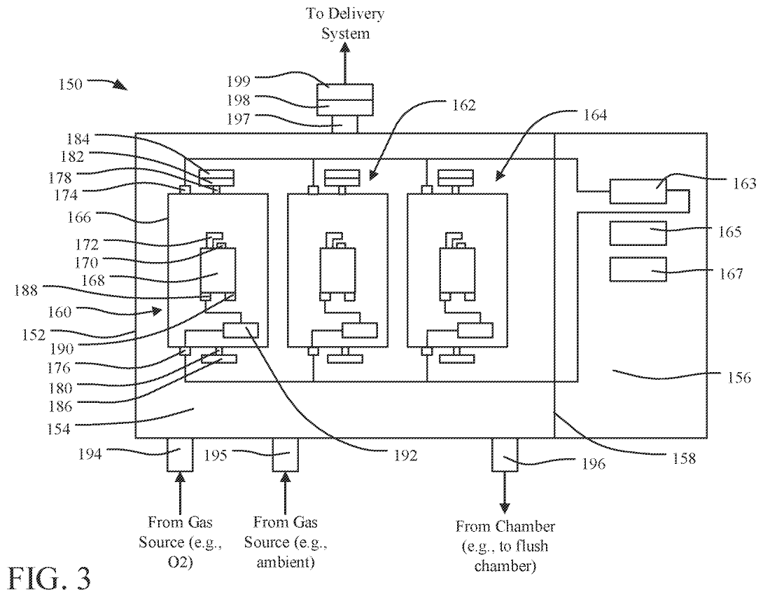

FIG. 3 shows a schematic illustration of a NO generator 150, which can be a specific implementation of the NO generators 118, 140. The NO generator 150 can include a housing 152 with compartments 154, 156 separated by a wall 158. The housing 152 can be fully (or partially) enclosed. For example, when the housing 152 is fully enclosed, an interior volume of the housing 152 is isolated from the ambient environment. The interior volume of the housing 152 can be separated by the wall 158 into the compartments 154, 156. In some cases, the compartments 154, 156 can be used to separate the components of the NO generator 150 from each other. For example, the compartment 154 can include NO generation units 160, 162, 164 positioned therein, while the compartment 156 can include a power source 163 (or multiple power sources), a controller 165, and a pump(s) 167 positioned therein. The controller 165 can be implemented in different ways. For example, the controller 165 can include typical components used such as a processor, memory, a display, inputs (e.g., a keyboard, a mouse, a graphical user interface, a touch-screen display, etc.), communication devices, etc. In some cases, the controller 165 can simply be implemented as a controller having a processor and memory. The controller 165 can communicate with other controllers and systems.

In some aspects, each NO generation unit 160, 162, 164 can be positioned within the interior volume of the housing 152, and in particular, the compartment 154 of the housing 152, and can be configured to generate NO gas from nitrogen gas and oxygen gas. The NO generation unit 160 can include an electrode housing 166, an park generator 168 having at least one pair of electrodes 170, 172 that are separated from each other by a gap, electrical connectors 174, 176, ports 178, 180, a scavenger 182, and filters 184, 186. The electrode housing 166 can define an interior volume that can be fully enclosed (or partially enclosed) and isolated from the interior volume of the housing 152 (e.g., the compartment 154). The electrical spark generator 168 can be positioned within the interior volume of the electrode housing 166, and thus can be isolated from the interior volume of the generator 150.

In some aspects, the electrical spark generator 168 can be removably coupled to the electrode housing 166 to selectively bring (and remove) the electrical spark generator 168 into (and out of) electrical connection with the power source 163 (e.g., via the electrode housing 166). For example, the electrical spark generator 168 can include electrical connectors 188, 190 that can respectively engage an electrical connector (or electrical conductor) of the electrode housing 166. In this way, when the electrical connectors 188, 190 contact a respective electrical connector of the electrode housing 166, the electrical connector 190 electrically connects to the electrical connector 174, while the electrical connector 188 electrically connects to the electrical connector 176. In this way, with the electrical connectors 174, 176 electrically connected to the power source 163, the electrical connectors 188, 190 and thus the electrodes 170, 172 are also electrically connected to the power source 163. The removably coupled configuration of the electrical spark generator 168 can be advantageous in that the electrical spark generator 168 can be easily removed and replaced without having to replace the entire system. In some configurations, the electrical spark generator 168 can include power circuitry 192 (e.g., which can include a spark coil, an amplifier(s), voltage regulators, a waveform generator, etc.) to provide the electrical power necessary to generate an electrical spark between the electrodes 170, 172. The power circuitry 192 can be electrically connected to the power source 163, via the electrical contact 176 of the electrode housing 166, and can be in selectively electrically connected to the electrical connector 188. In this way, when the electrical spark generator 168 is decoupled from the electrode housing 166 (e.g., removed for replacement), a different electrical spark generator 168 can be coupled to the electrode housing 166 thereby electrically connecting the power circuitry 192 to the electrical spark generator 168 via the electrical connector 188. Thus, replacement of the electrical spark generator 168 does not require replacement of the power circuitry 192.

In addition to the electrical spark generator 168, the electrode housing 166 can also be removably coupled to the housing 152 to selectively bring (and remove) the electrode housing 166 into (and out of) electrical connection with the power source 163. For example, when the electrode housing 166 is coupled to the housing 152, the electrical connector 174 contacts a first electrical conductor that is electrically connected to the power source 163, and the electrical connector 178 contacts and a second electrical conductor that is also electrically connected to the power source 163 thereby bringing the electrode housing 166 (and the electrical components therein) into electrical communication with the power source 163. In this way, the NO generation unit 160 can be easily removed to replace the NO generation unit 160 (e.g., when components of the NO generation unit 160 have reached the end of their usage life). In addition, such as when less NO concentration is desired to be delivered to the patient, the NO generation unit 160 can be removed.

In some aspects, the electrical connectors 174, 178, 188, 190 can be implemented in different ways. For example, the electrical connectors 174, 178, 188, 190 can each include a protrusion (or recess) that interfaces with a corresponding recess (or protrusion) to both secure the component and electrically connect the component. In some cases, each electrical connector 174, 178, 188, 190 can include a spring to ensure that the electrical connector remains is in contact with the corresponding electrical connector (or conductor), such as when the spring is biased.

Each port 176, 178 can be in fluid communication with the interior volume of the electrode housing 166 and can be in fluid communication with the interior volume of the housing 152. The port 176 can function as an input to direct gas into the interior volume of the electrode housing 166, while the port 178 can function as an output to direct gas out of the interior volume of the electrode housing 166. In some aspects, the filter 186, which can be structured in a similar manner as the filter 112, and can be removably coupled to the electrode housing 166 at the port 180. In this case, for example, when the filter 186 is coupled to the electrode housing 166 at the port 180, the filter 186 is brought in fluid communication with the port 180. In this way, gas received through the filter 186 is filtered prior to being directed into the interior volume of the electrode housing 166, which can prevent particulates from undesirably corroding the electrodes 170, 172. Similarly, the filter 184, which can also be implemented in a similar manner as the filter 112, can be removably coupled to the electrode housing 166 at the port 178. When the filter 184 is coupled to the electrode housing 166 at the port 178, gas within the interior volume of the electrode housing 166 passes through the filter 184 prior to being emitted into the interior volume of the housing 152. In some cases, the scavenger 182, which can be implemented in a similar manner as the scavengers 104, 110, can also be removably coupled to the filter 184 or the electrode housing 166. While the scavenger 182 is illustrated as being downstream of the filter 184, in other configurations, the scavenger 182 can be positioned upstream of the filter 184. Regardless of the configuration, the scavenger 182 can be in fluid communication with the port 178 so that gas that passes from the interior volume of the electrode housing 166 and into the interior volume of the housing 152 is removed of gaseous contaminants (e.g., NO₂). In this way, gaseous contaminants that are generated by the electrodes 170, 172 can be removed and prevented from passing into the interior volume of the housing 152.

In some aspects, the removably coupled configuration of the filters 184, 186 and the scavenger 182 is desirable at least because they can be easily replaced after being spent. For example, the scavenger 182 can change color after being spent (e.g., the reactive material being used up), and thus, when this occurs, the scavenger 182 can be decoupled from the filter 184 (or the electrode housing 166) and another scavenger 182 can be coupled thereto. In some aspects, each of the filters 184, 186, and the scavenger 182 can include a respective housing to secure and retain the component. In some cases, the filters 184, 186 can be movably engaged with a respective filter housing, while the scavenger 182 can be movably engaged with a respective scavenger housing. In this way, the respective components can be removed and replaced without having to decouple the respective housings.

In some aspects, each NO generation unit 160, 162, 164 can be structured in a similar manner, and thus the description of the NO generation unit 160 also pertains to the NO generation units 162, 164. For example, each of the NO generation units 162, 164 can include an electrode housing, an electrical spark generator, power circuitry, electrical connectors, ports, filters, a scavenger, etc. In some aspects, while each NO generation unit 160, 162, 164 are illustrated in FIG. 3 as including a respective electrode housing, in other configurations, some or all of the electrode housings can be removed, so that with an electrode housing removed the corresponding electrical spark generator is exposed to the interior volume of the housing 152. In some aspects, each NO generation unit 160, 162, 164 can be in communication with the controller 165, which can control operation of each NO generation unit 160, 162, 164. For example, the controller 165 can turn on (or off) each NO generation unit 160, 162, 164, and can adjust the frequency or amplitude of the power provided to the respective electrical spark generator of the respective NO generation unit 160, 162, 164. For example, the power circuitry of each NO generation unit 160, 162, 164 (e.g., the power circuitry 192 of the NO generation unit 160) can include an electrical switch (e.g., a transistor, relay, etc.) that can be opened or closed (e.g., by the controller 165) to selectively turn on (and off) power to the particular electrical spark generator. As another example, the controller 165 can adjust the amplitude of the power provided to an electrical spark generator by adjusting the gain of an amplifier of a power circuitry. As yet another example, the controller 165 can adjust the frequency of the power provided to an electrical spark generator by adjusting the frequency provided by a waveform generator of the power circuitry. Regardless of the configuration, the adjustability of the electrical spark generators by the controller 165 (and adjustability of power delivered to each electrical spark generator), can be desirable for generating different quantities of NO for different therapies. For example, the controller 165 can recruit additional electrical spark generators (or can increase the power amplitude and frequency for each electrical spark generator) for higher inhaled NO concentrations. Then, when lower inhaled NO concentrations are desired, the controller 165 can turn off (or decrease the power amplitude and frequency for each electrical spark generator).

In some aspects, the housing 152 can include ports 194, 195, 196, 197, which can each be in fluid communication with the interior volume of the housing 152. The ports 194, 195 can function as inputs and can each be in fluid communication with a gas source. For example, an O₂ gas source can be in fluid communication with the port 194 to emit O₂ gas into through the port 194 and into the interior volume of the housing 152, while an ambient air source can be in fluid communication with the port 195 to emit ambient air through the port 195 and into the interior volume of the housing 152. Conversely, the ports 196, 197 can function as outputs. For example, a pump (e.g., one of the pumps 167) can (periodically) drive gas out of the interior volume of the housing 152, through the port 196, and into a chamber (or into the ambient environment). As another example, the port 197 can also deliver gas from the interior volume of the housing 152, through the port 197, and to the other components downstream (e.g., via a pump, which can be one of the pumps 167).

In some aspects, the NO generator 150 can include a filter 198 and a scavenger 199. The filter 198 can be implemented in a similar manner as the previously described filters, and can be coupled to the housing 152 at the port 197 so that gas passing through the filter 198 is filtered. Similarly, the scavenger 199 can also be implemented in a similar manner as the other previously described scavengers, and can be coupled to the housing 152 at the port 197 (or the filter 198). In this way, gas passing through the scavenger 199 is removed of undesirable gaseous products. In some aspects, the filter 198 can be removably coupled to the housing 152 (or the scavenger 199), and the scavenger 199 can be removably coupled to the housing 152 (or the filter 198). In this way, the filter 198 and the scavenger 199 can be easily replaced. While the scavenger 199 is illustrated in FIG. 3 as being positioned downstream of the filter 198, in some configurations, the scavenger 199 can be positioned upstream of the filter 198. In some configurations, the NO generator 150 may include one of the scavenger 182 and the scavenger 199. For example, the scavenger 182 may be omitted and the scavenger 199 may be the sole scavenger in the NO generator 150.

In some aspects, the pump(s) 167 can be in communication with the power source 163, and thus the pump 167 can control each of the pumps 167. In some cases, such as when the pumps 167 are electrical pumps, the pumps 167 can be powered by the power source 163. Each port can include one of the pumps 167 to drive gas flow through the respective port. For example, a pump can drive gas flow through the port 194 and into the interior volume of the housing 152, a pump can drive gas flow through the port 196 and into the interior volume of the housing 152, a pump can drive gas flow through the port 198 and out of the interior volume of the housing 152, and a pump can drive gas flow through the port 197 and out of the interior volume of the housing 152. As another example, each NO generation unit 160, 162, 164 can include a pump at each port. In this case, a pump (of the pumps 167) can drive gas flow through the port 180 and into the interior volume of the electrode housing 166, and a pump (of the pumps 167) can drive gas flow through the port 178 and out of the interior volume of the electrode housing 166. In some cases, the housing of each pump 167 can be positioned within the compartment 156, while conduits that fluidly connect the particular components can be routed from the compartment 156.

In some aspects, the power source 163 can be implemented in different ways. For example, the power source 163 can be an alternating current (“AC”) source, or can be a direct current (“DC”) source. Ins some cases, the power source 163 can include other electrical components to appropriately deliver electrical power to the components (e.g., voltage regulator, a rectifier, inverters, transformers, etc.). In some aspects, the power source 163 can be an energy storage device (e.g., a battery). For example, the power source 163 can be a rechargeable battery (e.g., a lithium ion battery). In this way, for example, with the power source 163 being a rechargeable battery the NO generator 150 can be more portable as it is less reliant on local power sources (e.g., power outlets). Then, periodically, the rechargeable battery can be recharged (e.g., when close to a power source). In some aspects, while one power source 163 is illustrated in FIG. 3, in alternative configurations, the NO generator 150 can include multiple power sources, with each power source powering a respective electrical spark generator.

In some aspects, the NO generator 150 can include sensors (not shown) that can be in communication with the controller 165 and can facilitate better NO generation (and undesirable byproduct removal). For example, the NO generator 150 can include NO sensors, NO₂ sensors, O₂ sensors, flow sensors, pressure sensors, a blood oxygenation sensor, a methemoglobin sensor, a blood oxygenation sensor, a blood pressure sensor, a heart rate sensor, an ECG, etc.

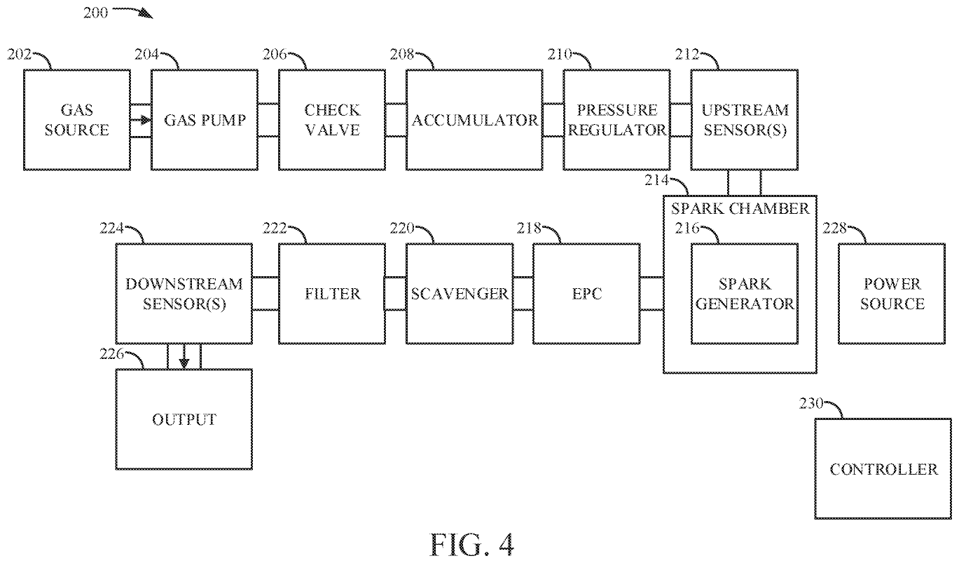

FIG. 4 shows a schematic illustration of an NO generator 200, which can be a specific implementation of the NO generators 118, 140, 150. The NO generator 200 can include a gas source 202, a gas pump 204, a check valve 206, an accumulator 208, a pressure regulator 210, upstream sensors 212, a spark chamber 214, a spark generator 216, an electronic pressure control unit 218 (i.e., an “EPC”), a scavenger 220, a filter 222, downstream sensors 224, an output 226, a power source 228, and a controller 230. The controller 230 can be implemented in different ways. For example, the controller 230 can include typical components used such as a processor, memory, a display, inputs (e.g., a keyboard, a mouse, a graphical user interface, a touch-screen display, etc.), communication devices, etc. In some cases, the controller 230 can simply be implemented as a controller having a processor and memory. The controller 230 can communicate with other controllers and systems.

The gas pump 204 can be positioned downstream of the gas source 202 and can drive downstream flow of gas from the gas source 202. The gas pump 204 can be implemented similarly to the other previously described pumps. The check valve 206 is positioned downstream of the gas pump 204 and can block upstream flow of gas from the gas source 202, while allowing downstream flow of gas. The accumulator 208 can be positioned downstream of the check valve 206 (and the gas pump 204), and similarly to the accumulator of the NO generator 140, is configured to store pressurized gas (e.g., maintain the pressure of the gas). In some configurations, adjusting the flow rate (e.g., by adjusting the speed of the gas pump 204) can adjust the pressure delivered to the accumulator 208.

The pressure regulator 210 is positioned downstream of the accumulator 208 (and of the gas pump 204) and can regulate the pressure of gas delivered to the spark chamber 214. For example, the pressure regulator 210 can output a pressure that is lower than the pressure received by the accumulator 208, but which is maintained at a substantially constant output pressure (e.g., largely free of pressure fluctuations) due to the inclusion of the accumulator 208. In some cases, the output pressure of the pressure regulator 210 can be adjustable (e.g., by a controller 230). In this way, the output pressure provided by the pressure regulator 210 can be selectively adjusted, for example, depending on the desired NO gas concentration to be generated. The NO generator 200 can include upstream sensors 212 (e.g., sensors upstream of the scavenger 220). The upstream sensors 212 can include a pressure sensor, a flow sensor, an O₂ sensor, an N₂ sensor, each of which can measure a characteristic of the gas flow delivered to the spark chamber 214.

The spark chamber 214 is positioned downstream of the pressure regulator 210, and can include an electrical spark generator 216 (or multiple electrical spark generators). The spark chamber 214 is provided with pressurized gas (from the accumulator 208 and the pressure regulator 210) at an inlet of the spark chamber 214, while gas including NO gas, is emitted out through the outlet of the spark chamber 214 to the EPC 218. The EPC 218, which can be positioned downstream of the spark chamber 214, can be configured to maintain a constant output pressure, and can be controlled by the controller 230. For example, the controller 230 can cause the EPC 218 to provide a constant output pressure to components downstream of the EPC 218. The scavenger 220 can be positioned downstream of the EPC 218 and receives gas including NO gas at the output pressure provided by the EPC 218. The scavenger 220 can be implemented similar to the previously described scavengers, and thus can remove undesirable gaseous products from the gas including NO₂. In some cases, the scavenger 220 being positioned downstream of the EPC 218 is advantageous in that the EPC 218 can provide an output pressure that is lower than the input pressure into the spark chamber 214. In this way, the lower pressure can be better for passage through the scavenger 220 (e.g., with a lower flow rate a larger percentage of gas is exposed to the scavenger 220 for a longer period of time), while higher pressures can be advantageous for NO generation in the spark chamber 214.

The filter 222 is positioned downstream of the scavenger 220, and can be implemented in a similar manner as the previously described filters. The downstream sensors 224 can be positioned downstream of the scavenger 220 and can include a NO senor, a NO₂ sensor, a pressure sensor, a flow sensor, etc., each of which can sense parameters of the gas outputted from the output 226 for control of the NO generator 200 (e.g., as described above with regard to FIG. 2). The power source 228 can be electrically connected to each of the components of the NO generator 200, as appropriate, including the gas pump 204, the pressure regulator 210 (e.g., when the pressure regulator 210 is an electric pressure regulator), the upstream sensors 212, the electric spark generator(s) 216, the EPC 218, the downstream sensor(s) 224 and the controller 230. The controller 230 can be in communication with some or all of the components of the NO generator 200, as appropriate, including the gas pump 204, the pressure regulator 210, the spark generator(s) 216, the EPC 218, the downstream sensors 224, etc. In this way, the controller 230 can receive data from and control aspects of each component in communication with the controller 230.

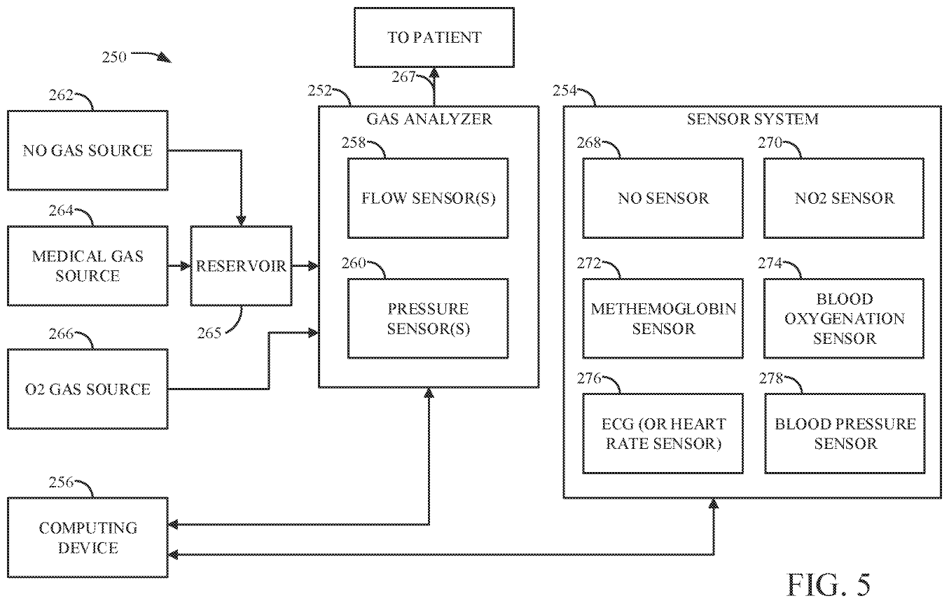

FIG. 5 shows a schematic illustration of an NO delivery sensor system 250. The NO delivery sensor system 250 can include a gas analyzer 252, a sensor system 254, and a controller 256. The controller 256 can be implemented in different ways. For example, the controller 256 can include typical components used such as a processor, memory, a display, inputs (e.g., a keyboard, a mouse, a graphical user interface, a touch-screen display, etc.), communication devices, etc. In some cases, the controller 256 can simply be implemented as a controller having a processor and memory. The controller 256 can communicate with other controllers and systems.

The gas analyzer 252 can include flow sensors 258, and pressure sensors 260. For example, the flow sensors 258 can include at least three flow sensors with a first flow sensor sensing the flow rate of gas delivered by an NO gas source 262, a second flow sensor sensing the flow rate of gas delivered by a medical gas source 264, and a third flow sensor sensing the flow rate of gas delivered by an O₂ gas source 266. Similarly, the pressure sensors 260 can include at least three pressure sensors with a first pressure sensor sensing the pressure of the gas delivered by the NO gas source 262, a second pressure sensor sensing the pressure of the gas delivered by the medical gas source 264, and a third pressure sensor sensing the pressure of the gas delivered by the O₂ gas source. In some configurations, the NO gas source 262 may be one of the NO generator 118, the NO generator 140, the NO generator 150, or the NO generator 200. The NO gas source 262 and the medical gas source 264 may combine into a reservoir 265. The reservoir 265, similar to the reservoir 108, can add a predetermined amount of volume the flow path between the NO gas source 262 and an outlet 267 in fluid communication with a patient. In this way, the reservoir 265 can contain and maintain a substantially constant concentration of NO so that NO gas delivery to the patient is regulated (e.g., does not fluctuate beyond a predetermined tolerance of a particular concentration). In some aspects, the predetermined tolerance may be less than about plus or minus 5%, less than about plus or minus 10%, or less than about plus or minus 20%. In some aspects, the reservoir 108 can define a volume of greater than about 0.25 liters (L), greater than about 0.5 L, greater than about 1 L, greater than about 2 L, or about 3 L.

The sensor system 254 can include multiple sensors that can sense parameters to control components of an NO delivery system. For example, the sensor system 254 can include an NO sensor 268, a NO₂ sensor 270, a methemoglobin sensor 272, a blood oxygenation sensor 274, an ECG 276, and a blood pressure sensor 278. The NO sensor 268 and the NO₂ sensor 270 can each be in fluid communication with a conduit that delivers NO gas to a subject. For example, the NO sensor 268 and the NO₂ sensor 270 can be positioned within the conduit. The NO sensor 268 and the NO₂ sensor 270 can each be implemented in a similar manner as the previously described NO sensors NO₂ sensors, respectively. In some cases, the NO sensor 268 and the NO₂ sensor 270 can sense parameters that can control aspects of an NO delivery system. For example, the NO sensor 268 can sense the NO concentration, which can be used (e.g., by the controller 256) to increase the flow rate of gas delivered by the NO gas source 262, to decrease the flow rate of gas delivered by the medical gas source 264, or to decrease the flow rate of gas delivered by the O₂ gas source 266. As another example, the NO₂ sensor 270 can sense the concentration of NO₂, which can be used (e.g., by the controller 256) to decrease the flow rate of gas delivered by the NO gas source 262, to increase the flow rate of gas delivered by the medical gas source 264, or to increase the flow rate of gas delivered by the O₂ gas source 266.

In some cases, the sensor system 254 can include patient based sensors, which can sense patient parameters to control aspects of the NO delivery system. For example, the patient based sensors can include the methemoglobin sensor 272, the blood oxygenation sensor 274, the ECG 276, and the blood pressure sensor 278. The methemoglobin sensor 272 is configured to sense the concentration of methemoglobin circulating in the patient's blood stream, and can be implemented in different ways. For example, the methemoglobin sensor 272 can be a pulse co-oximeter. The methemoglobin concentration sensed by the methemoglobin sensor 272 can be used (e.g., by the controller 256) to control aspects of the NO delivery system including decreasing (or stopping altogether) the flow rate of gas delivered by the NO gas source 262, increasing the flow rate of gas delivered by the medical gas source 264, or increasing the flow rate of gas delivered by the O₂ gas source 266. Similarly, the other patient based sensors including the blood oxygenation sensor 274, the ECG 276, and the blood pressure sensor 278 can each control aspects of the NO delivery system in a similar manner as the methemoglobin sensor 272. For example, the blood oxygenation sensor 274 can sense the blood oxygenation level of within the blood stream of the patient, the ECG 276 can sense the heart rate of the patient, and the blood pressure sensors 278 can sense the blood pressure of the patient, each of which can be used (e.g., by the controller 256) to decrease (or to stop altogether) the flow rate of gas delivered by the NO gas source 262, increase the flow rate of gas delivered by the medical gas source 264, or increase the flow rate of gas delivered by the O₂ gas source 266. In some aspects, the sensor system 254 can include a respiratory rate sensor, which can sense the respiratory rate of a patient, and which can be used to control aspects of the NO delivery system.

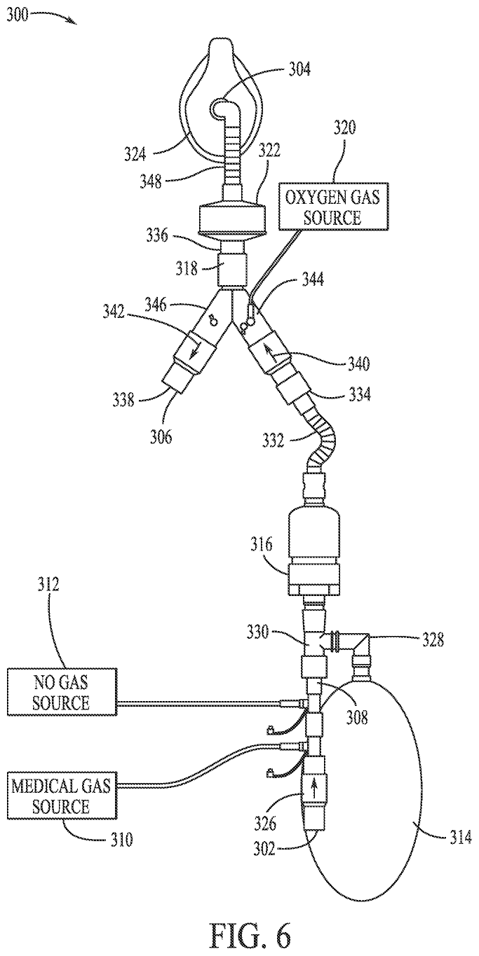

FIG. 6 shows a schematic illustration of an NO delivery system 300, which can be a specific implementation of the NO delivery system 100. The NO delivery system 300 can define an inlet 302 that allows gas to travel into an through the NO delivery system 300, and outlets 304, 306 that allow gas to travel out of the NO delivery system 300 and into the ambient environment. For example, the outlet 304 may be a first outlet that is configured to be placed in communication with a patient, and the outlet 306 may be a second outlet in communication with the ambient environment. The NO delivery system 300 can include a main conduit 308, a medical gas source 310, an NO gas source 312, a reservoir 314, a scavenger 316, a three port connector 318, an oxygen gas source 320, a filter 322, and a patient interface 324. In some configurations, the NO gas source 312 may be one of the NO generator 118, the NO generator 140, the NO generator 150, or the NO generator 200. The medical gas source 310 (e.g., air or O₂) and the NO gas source 312 are each in fluid communication with the main conduit 308, and each are positioned downstream of the inlet 302. However, in alternative configurations, the inlet 302 can be directly connected to the medical gas source 310 (e.g., via tubing) so that medical air is delivered through the inlet 302. In some cases, the inlet 302 can be sealed from the ambient environment so that medical air and not ambient air is only delivered through the inlet 302. In some configurations, the NO gas source 312 is fluidly connected to the main conduit 308 downstream of the location that the medical gas source 310 is fluidly connected to the main conduit 308. However, in other configurations, the medical gas source 310 can be fluidly connected to the main conduit 308 downstream of the location that the NO gas source 312 is fluidly connected to the main conduit 308.

In some aspects, the NO delivery system 300 can include a one-way valve 326 that is positioned within the main conduit 308 and downstream of the inlet 302. In particular, the one-way valve 326 can be positioned between the inlet 302 and the location in which the medical gas source 310 is fluidly connected to the main conduit 308, and between the inlet 302 and the location in which the NO gas source 312 is fluidly connected to the main conduit 308. The one-way valve 326 can be configured to allow downstream flow through the NO delivery system 300, while blocking upstream flow through the NO delivery system 300. As shown in FIG. 3, the reservoir 314 is in fluid communication with the main conduit 308 and is positioned downstream of the inlet 302, the medical gas source 310, and the NO gas source 312. In addition, the reservoir 314 can be oriented so that an axis defined by opposing ends of the reservoir 314 is substantially (i.e., deviating by less than 20% from) parallel to a flow path through the NO delivery system 300 defined from the inlet 302 and to the location in which the reservoir 314 is fluidly connected to the main conduit 308. In some cases, the NO delivery system 300 can include an elbow connector 328, which has a first end coupled to the main conduit 308 at a location 330 and a second end opposite the first end coupled to an opening of the reservoir 314. Although the elbow connector 328 is illustrated as having a substantially 90 degree bend between the opposing ends, in other configurations, the elbow connector 328 can have other bend angles.

In some aspects, and as illustrated, the reservoir 314 extends entirely in an upstream direction from the location 330 (e.g., no portion of the reservoir 314 extends downstream of the location 330). In some cases, the reservoir 314 can extend in an upstream direction past the inlet 302 (e.g., when the reservoir 314 is inflated). The reservoir 314 is shown as being expandable, and thus the reservoir 314 can be formed out of elastic materials (e.g., polymers, rubber, etc.) that can be selectively expanded and retracted based on the amount of gas positioned within the reservoir 314. The reservoir 314 can add a predetermined amount of volume the flow path between the inlet 302 and the outlet 304 (e.g., the first outlet) in fluid communication with a patient. In this way, the reservoir 314 can contain and maintain a substantially constant concentration of NO so that NO gas delivery to the patient is regulated (e.g., does not fluctuate beyond a predetermined tolerance of a particular concentration). In some aspects, the predetermined tolerance may be less than about plus or minus 5%, less than about plus or minus 10%, or less than about plus or minus 20%. In some aspects, the reservoir 314 can define a volume of greater than about 0.25 liters (L), greater than about 0.5 L, greater than about 1 L, greater than about 2 L, or about 3 L.

The reservoir 314 is advantageously positioned downstream of both the NO gas source 312, and the medical gas source 310 so that NO gas (from the NO gas source 312, and medical air (from the medical gas source 310) fill the reservoir 314 with a (predetermined) concentration of NO gas, which can be determined by the gas flow delivered to the main conduit 308 from the NO gas source 312, from the medical gas source 310, and from the inlet 302 (e.g., ambient air flow, if applicable, through the inlet 302). In this way, the NO gas delivered to the patient through the outlet 304 remains at a substantially constant NO concentration and is less prone to NO concentration fluctuations (e.g., as would be otherwise without the reservoir 314 filled with NO gas).

As shown in FIG. 6, the scavenger 316, which can be implemented in a similar manner as the previously described scavengers, can be positioned upstream of the reservoir 314, and in particular, upstream of the location 330. In this way, as gas is delivered from the reservoir 314 and downstream through the main conduit 308, the gas is passed through the scavenger 316 and is removed of undesirable gaseous products. In some cases, the scavenger 316 can define a portion of the main conduit 308. Thus, the scavenger 316 can be positioned within the flow path of the main conduit 308. In some aspects, the cross-sectional area of the main conduit 308 at the scavenger 316 can be larger than the cross-sectional area of other portions of the main conduit 308 (e.g., the cross-sectional area of the inlet 302). In some cases, the cross-sectional area of the main conduit 308 can be larger than the cross-sectional area of a portion of the main conduit 308 upstream of the scavenger 316 and a portion of the main conduit 308 downstream of the scavenger 316. In this way, the gas can be exposed to a larger surface area to increase the removing ability of the scavenger 316 (e.g., rather than a scavenger with a lower surface area the size of the portions of the main conduit 308 upstream or downstream of the scavenger 316). In some aspects, the scavenger 316 can include calcium hydroxide.

In some aspects, the NO delivery system 300 can include a flexible tube 332 positioned upstream of the scavenger 316. The flexible tube 332 can also define a portion of the main conduit 308. The flexible tube 332 can be coupled to the scavenger 316 (and other components that are coupled thereto), and allows the scavenger 316 to adjust its position and orientation relative to an end of the flexible tube 332 (e.g., the most downstream end of the flexible tube 332). For example, the flexible tube 332 can have one or more curves with different radii of curvature to accommodate for the change in positioning and orientation of the scavenger 316 and other components. As a specific example, the flexible tube 332 can have a serpentine shape.

The three port connector 318 can be positioned upstream of the scavenger 316 and upstream of the flexible tube 332. The three port connector 318 can define ports 334, 336, 338, some of which can define the main conduit 308. For example, the ports 334, 336 can further define the main conduit 308, while the port 338 branches off of the main conduit 308. In some aspects, the NO delivery system 300 can include one-way valves 340, 342, each of which can be positioned within the three port connector 318. For example, the one-way valve 340 can be an inspiratory valve positioned in a first branch 344 of the three port connector 318 between the ports 334, 336, while the one-way valve 342 can be a expiratory valve positioned in a second branch 346 of the three port connector 318 between the ports 336, 338. The one-way valve 340 can be configured to allow downstream flow through the NO delivery system 300, while blocking upstream flow through the NO delivery system 300. Conversely, the one-way valve 342 can be configured to allow upstream flow through the NO delivery system 300, while blocking downstream flow through the NO delivery system 300. Thus, during inhalation gas flows in a downstream direction through the NO delivery system 300, passing through the one-way valve 340, and is prevented from flowing from the ambient environment and through the one-way valve 342 in a downstream direction. Conversely, during exhalation, gas flows in an upstream direction through the one-way valve 342 and out into the ambient environment, while gas flow is blocked from flowing in a downstream direction through one-way valve 340. In some aspects, and as illustrated the three port connector 318 can have a y-shape.

The oxygen gas source 320 can be in communication with the first branch 344 at a location downstream of the valve 344 (e.g., the inspiratory valve). Introducing the oxygen gas close to the patient (e.g., downstream of the valve 344) allows less time for the oxygen to react with the NO to form undesirable NO₂. In other words, this allows for simultaneous oxygen gas and NO gas delivery to a patient, in which oxygen gas delivery can be particularly important for patients that have pneumonia or COVID-19, or other conditions in which gas exchange in the lungs has been worsened.

The filter 322 can be implemented in similar ways as the previously described filters. For example, the filter 322 can be a HEPA filter. The filter 322 is positioned downstream of the three port connector 318. In some cases, the filter 322 can have a larger cross-section than all the cross-sections of the three port connector 318. For example, the filter 322 can have a larger cross-section compared to the cross-section of the main conduit 308 upstream of the filter 322, and the cross-section of the main conduit 308 downstream of the filter 322. In this way, a larger surface area of the filter 322 is exposed to filter the gas flowing along the main conduit 308, and at the same time the increase in cross-section advantageously decreases the flow rate of gas flowing through the filter 322, which can better filter particulars out of the gas. In some cases, a flexible connector 348 can be positioned downstream of the filter 322 and can be situated between the filter 322 and the patient interface 324. The flexible connector 348 can also define a portion of the main conduit 308. The flexible connector 348 can advantageously allow for the adjustment in orientation and position of the patient interface 324 relative to other components of the NO delivery system 300 including the filter 322 and components upstream of the filter 322.

The patient interface 324 can be configured to engage the face, the mouth, etc., of the patient to deliver gas including NO gas to the respiratory system of the patient. For example, as shown in FIG. 6, the patient interface 324 can be a mask, however, in alterative configurations the patient interface 324 can be a mouthpiece that is inserted into the oral cavity for the subject. In some cases, the patient interface 324 can be a cannula that is inserted into the nasal cavity of patient, can include a nasal pillow(s) (e.g., a pair of nasal pillows) that are each inserted into a nostril of a patient, or can be an intratracheal catheter (e.g., an intratracheal non-kinking scoop catheter). While not shown, the patient interface 324 can include other features to couple the patient interface 324 to a portion of the head of the patient, including straps, bands, clasps, clips, etc.

As shown in FIG. 6, the patient interface 324 can define the outlet 304 of the NO delivery system 300 so that NO gas is delivered through the outlet 304 and into the respiratory system of the patient. In operation, with the patient interface 324 secured to the patient's head, inspiration by the patient (e.g., generating a negative pressure drives downstream gas flow from the NO gas contained within the reservoir 314 to and through the outlet 304 delivering NO gas to the patient. In addition, during inspiration, ambient air flows downstream through the one-way valve 326, mixing with the medial air, and the NO gas (from the NO gas source) and is delivered to the reservoir 314. During exhalation, however, positive pressure is generated, driving upstream gas flow from the patient's respiratory system through the outlet and through the one-way valve 342 and out the outlet 306 into the ambient environment. Because upstream flow is blocked by the one-way valve 340, during expiration, upstream gas flow cannot flow to past the one-way valve 340. This inspiration and expiration cycle repeats until the NO treatment session has been completed.

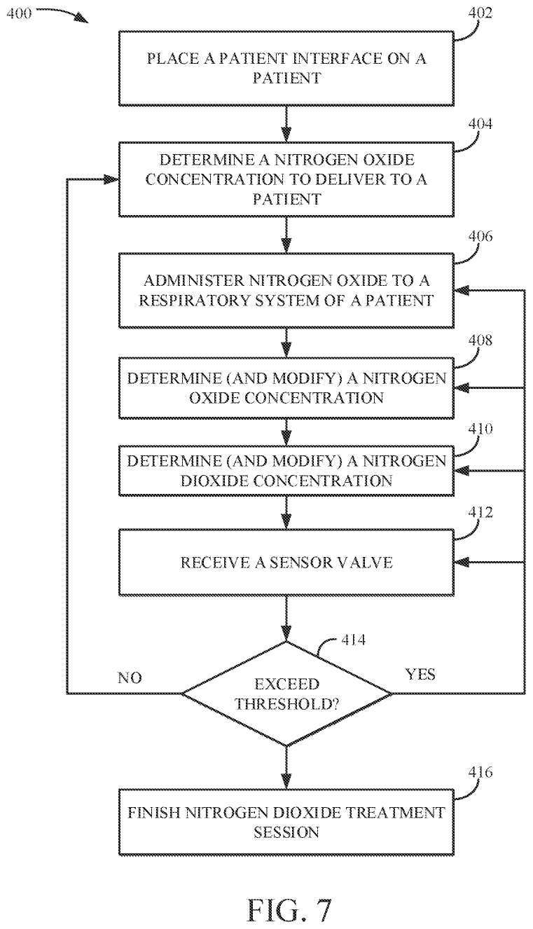

FIG. 7 shows a flowchart of a process or method 400 for generating NO gas and delivering the NO gas to a patient. The method 400 can be implemented using any of the previously described NO delivery systems, NO generators, or any of the systems described in the examples section herein. In addition, some or all of the blocks of the method 400 can be implemented using one or more controllers, as appropriate. In some aspects, the method 400 can be implemented without the aid of a positive pressure generating apparatus including a CPAP device, a ventilator, etc. In other words, the method 400 can be implemented so that the patient themselves drive inspiration and expiration of the NO gas.

At 402 the method 400 can include placing a patient interface on a patient. In some cases, this can include securing a patient interface to a head of the patient (or other portion of the patient).

At 404, the method 400 can include a controller determining an NO concentration to deliver to a patient. In some cases, this can include a controller receiving a user input that is indicative of the NO concentration to deliver to a patient. In some cases, a controller can determine the NO concentration based on a parameter of the patient, including a type of infection (e.g., a viral, a bacterial, a fungal, etc., and specific pathogens within each category), a severity of an infection (e.g., a fever being above a particular temperature, a blood oxygenation level being below a threshold, a respiratory rate being below a threshold, etc.). In some cases, the NO concentration can be based on a desired FiO₂ level to be administered to the patient, including if the FiO₂ level to be delivered is higher than a threshold (e.g., 0.21).

In some aspects, the NO concentration can be a high NO concentration, including being higher than 100 ppm. In some specific cases, the NO concentration can be greater than 150 ppm (e.g., within a range of that is substantially 150 ppm to substantially 300 ppm).

At 406, the method 400 can include administering NO at the determined concentration to the respiratory system of the patient (e.g., via the patient interface). In some cases, this can include a controller causing a NO generator to generate NO dioxide at the determined NO concentration, or at an NO concentration that is higher than the determined NO concentration (e.g., so that the NO concentration delivered by the NO generator can be diluted to the determined NO concentration). For example, a controller can adjust the pressure delivered to a NO generation chamber (e.g., that houses the one or more electrical spark generators), which can be done by the controller adjusting the speed of a pump that delivers gas to an accumulator upstream of the NO generation chamber, or by adjusting the output pressure of a pressure regulator that supplies gas to the NO generator chamber. As another example, a controller can adjust the power supplied to the electrical spark generator (e.g., by adjusting the pulse width modulation (“PWM”) of the electrical signal delivered to the electrical spark generator, the number of electrical pulses delivered to the electrical spark generator per second). As yet another example, a controller can increase the number of electrical spark generators receiving power (e.g., recruiting, or removing from recruitment electrical spark generators).

In some aspects, at the block 406, the method 400 can include administering O₂ to the patient from an O₂ source (e.g., that is separate from the NO source, which can be an NO generator), and administering medical air to the patient. In some cases, this can include mixing medical air with NO gas produced by the NO generator prior to delivery to the patient. For example, this can include diluting NO gas produced by the NO generator with a gas (e.g., medical air) until the NO gas reaches the determined NO concentration.

At 408, the method 400 can include determining an NO concentration. For example, a controller can receive, from an NO sensor, an NO concentration. Then, in some cases, a controller can determine whether or not an NO concentration exceeds a threshold (e.g., the determined NO concentration at the block 404, or an NO concentration range that includes the determined NO concentration). If the controller determines that the NO concentration exceeds the threshold, the method 400 can include a controller modifying a current NO concentration. In some cases, this can include a controller causing the NO generator to decrease (or increase) the NO generation, causing an O₂ gas source to increase (or decrease) in flow (e.g., by the controller adjusting the opening of a valve, adjusting the speed of a pump, etc.), causing an increase (or decrease in gas delivered by a gas source that mixes with the generated NO gas. If the controller determines that the NO concentration is within the threshold, in some cases, the controller can make no adjustments.

At 410, the method 400 can include a controller determining an NO₂ concentration. For example, a controller can receive, from a NO₂ sensor, an NO₂ concentration. Then, a controller can determine whether or not the NO₂ concentration exceeds (e.g., is greater than) a threshold (e.g., a concentration of substantially 1 ppm). If the controller determines that the NO₂ concentration exceeds the threshold, the method 400 can include a controller modifying a current NO₂ concentration. In some cases, this can include a controller causing the NO generator to decrease the NO generation (e.g., decrease the generated concentration of NO), causing an O₂ gas source to increase in flow (e.g., by the controller increasing the opening of a valve, increasing the speed of a pump, etc.), causing an increase in gas delivered by a gas source that mixes with the generated NO gas. In some cases, this can include a controller stopping flow of NO from an NO gas source (e.g., by closing a valve), or decreasing flow of NO from an NO gas source (e.g., by closing a valve by a particular amount, decreasing a speed of a pump, etc.). If the controller determines that the NO₂ concentration is within the threshold, in some cases, the controller can make no adjustments.

At 412, the method 400 can include a controller receiving a sensor value. In some cases, this can include a controller receiving a methemoglobin concentration from a methemoglobin sensor, receiving a blood oxygenation value from a blood oxygenation sensor, receiving a heart rate value from a heart rate sensor (e.g., an ECG), receiving a blood pressure value from a blood pressure sensor (e.g., a mean arterial pressure, a right ventricular systolic pressure, etc.), a respiratory rate sensor from a respiratory rate sensor, etc.

At 414, the method 400 can include a controller determining whether or not the sensor value exceeded a threshold. For example, a controller can determine whether or not a methemoglobin concentration exceeds a threshold value (e.g., when the sensor value is a methemoglobin concentration). If at the block 414, a controller determines that the sensor value exceeded the threshold, the method 400 can proceed back to any of blocks 406, 408, 410, 412. For example, the method 400 can proceed back to the block 408 to modify the current NO concentration, which can include a controller decreasing the NO concentration (or stopping NO delivery altogether). If, however, at the block 414 the controller determines that the sensor value has not exceeded the threshold, the method 400 can proceed back to the block 404.

While the method 400 could, in theory, operate in an infinite loop, the method 400 can proceed to block 416 to finish the NO treatment session, based on, for example, the treatment duration being elapsed for a desired duration.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

### Example 1

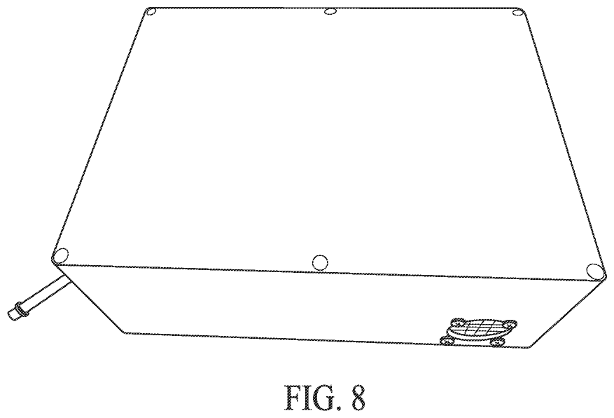

FIG. 8 shows an example of a high-pressure electric NO (“eNO”) generator with a size and a weight. The eNO generator has a length of 30 cm, a width of 23 cm, and a height of 11 cm. The weight of the eNO generator is 11.6 lbs (5.3 kg).

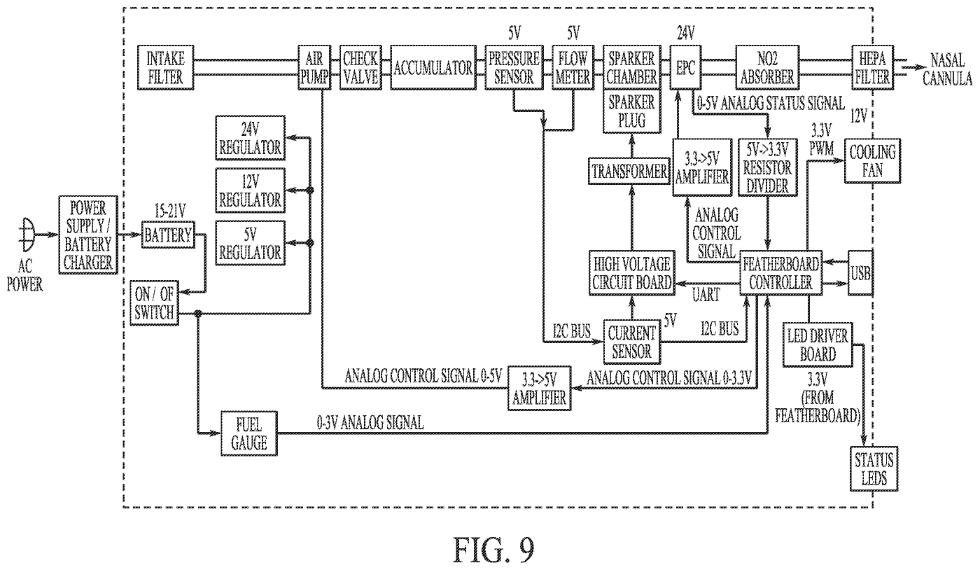

FIG. 9 shows a block diagram of a high pressure eNO generator.

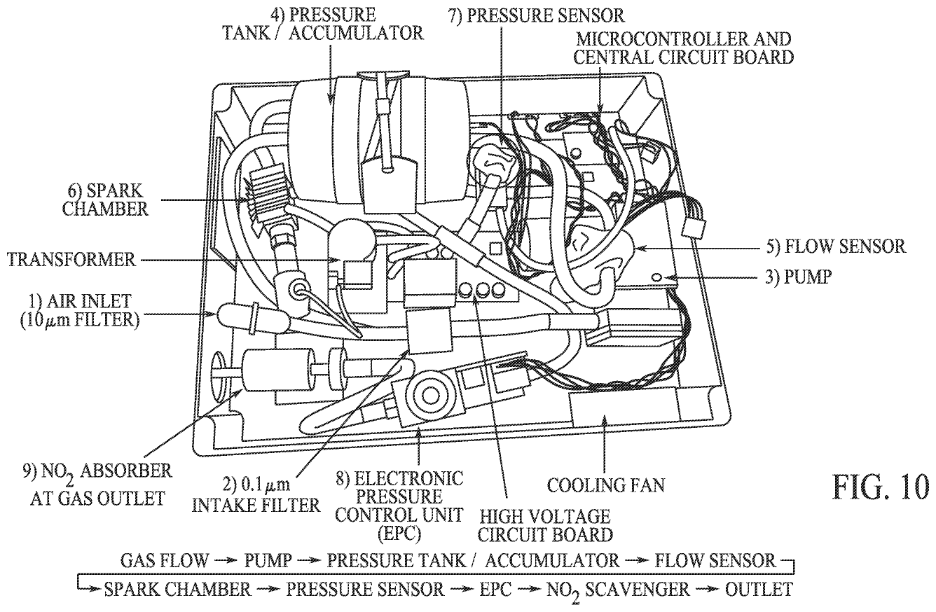

FIG. 10 shows a schematic of the inner components of the high-pressure eNO generator.

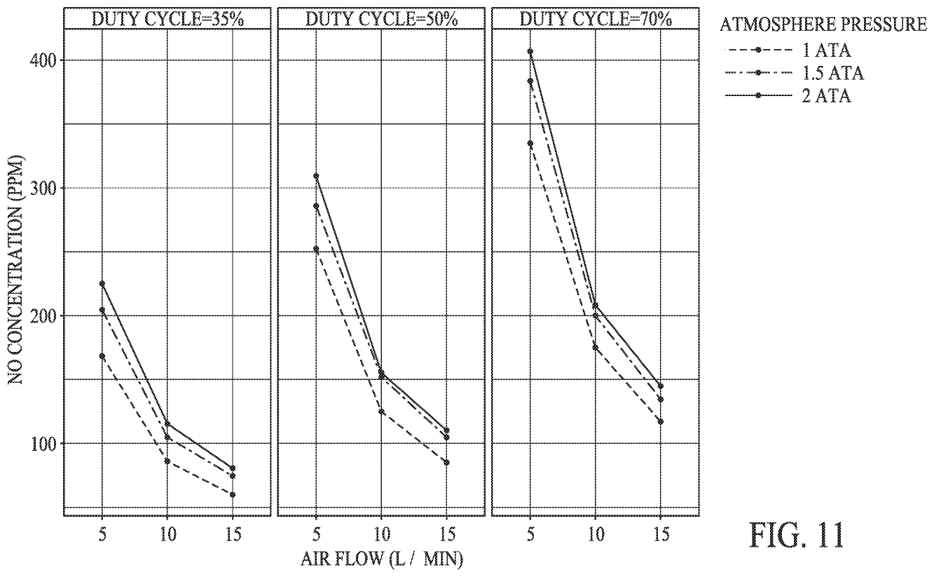

FIG. 11 shows three graphs of NO concentration (ppm) verses air flow rate (L/min) for different duty cycles of the power delivered to the electrical spark generator and for different pressures within the spark chamber. The first graph used a duty cycle of 35%, the second graph used a duty cycle of 50%, and the third graph used a duty cycle of 70%. The setup included a flow rate of gas into the NO generator at 1.5 L/min, a cooling fan at 50% of its maximum speed, and a pulse rate of 5 ms for the duty cycles. The graphs show that higher atmospheric pressure (and lower air flow rates at the output of the NO generator) enhances the production of NO.

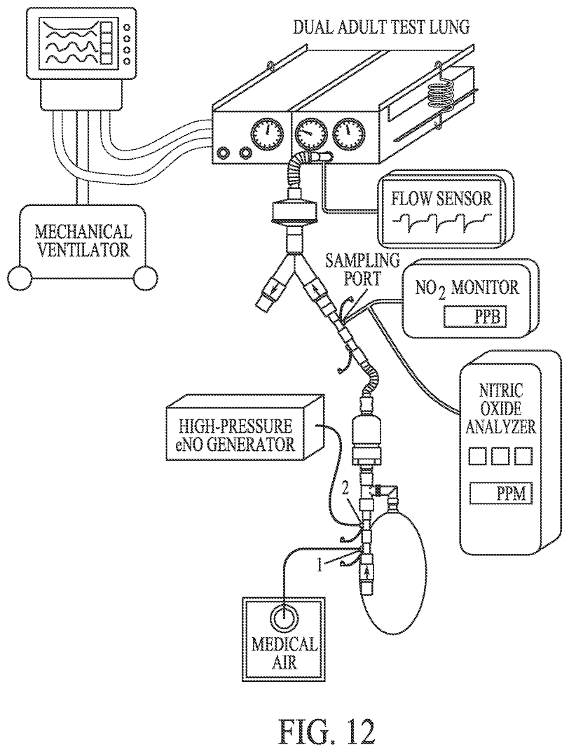

FIG. 12 shows a schematic of an experimental setup.

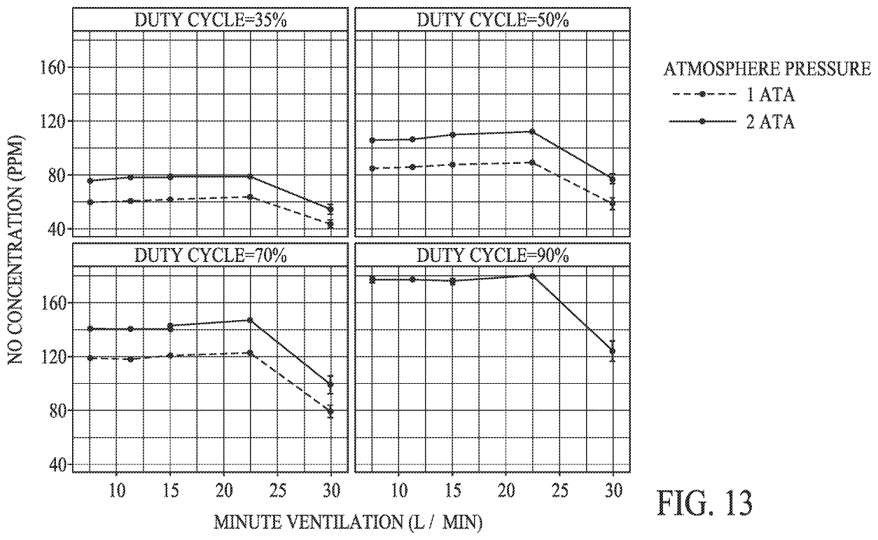

FIG. 13 shows four graphs of NO concentration (ppm) vs. minute ventilation (L/min) for different pressures within the NO generator, and for different duty cycles of power delivered to the electrical spark generator of the NO generator. These graphs show that increasing the atmospheric pressure and duty cycle of sparking enhance NO production. The NO₂ levels for all of these were below 1 ppm for all NO concentrations listed.

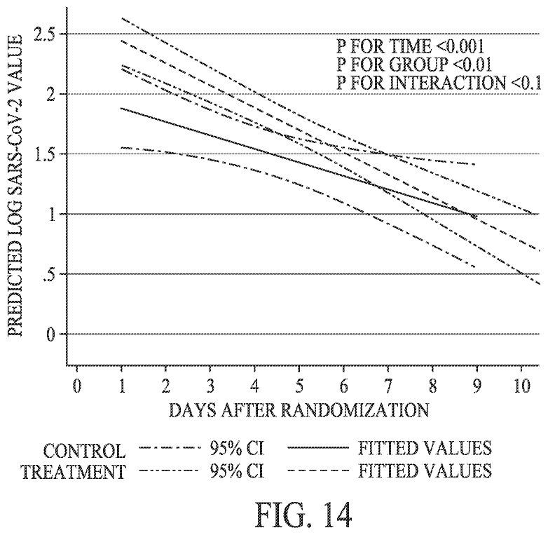

FIG. 14 shows a graph of the predicted log SARS-COV-2 value vs. the days after randomization for treatment and control groups. The viral load was decreased in the blood of intubated patients with severe COVID-19 in ICU and treated with inhalation of NO. Control: n=16, standard of care. Treatment (open label): n=16, patients inhaled 80 ppm of NO (diluted from NO/N₂ tank) for 48-hr, and NO was reduced to 40 ppm if/when PaO₂/FiO₂ was higher than 300 mmHg.

Table 1 shows the NO production at various atmospheric pressures with different electrode gaps for a continuous air flow of 3 L/min.

TABLE 1
NO production at various atmospheric pressures
with different electrode gaps

| Sea-level Atmospheric | Electrode | NO production |
|---|---|---|
| pressure (ATA) | gap (mm) | (ppm) |
| 1 | 1 | 30 ± 1 |
| 1.5 | | 33 ± 2 |
| 1 | 2 | 80 ± 1 |
| 1.5 | | 127 ± 1 |
| 1 | 3 | 120 ± 2 |
| 1.5 | | 245 ± 2 |

Table 2 shows the NO production and power consumption (voltage and current) at various atmospheric pressures with a continuous air flow of 3 L/min.

TABLE 2
NO production and power consumption
at various atmospheric pressures

| Sea-level | | | |
|---|---|---|---|
| Atmospheric | NO production | Voltage | Current |
| Pressure (ATA) | (ppm) | (kV) | (Amp) |
| 1 | 80 | 2-2.5 | 0.18-0.3 |
| 1.5 | 127 | 2.5-2.8 | 0.22-0.31 |

In summary, the high-pressure electrical NO generator continuously delivered NO at relatively high NO concentrations. Increasing atmospheric pressure enhances NO production, and inhalation of NO decreases viral load in the blood of intubated patients with severe Covid-19 in the ICU

### Example 2

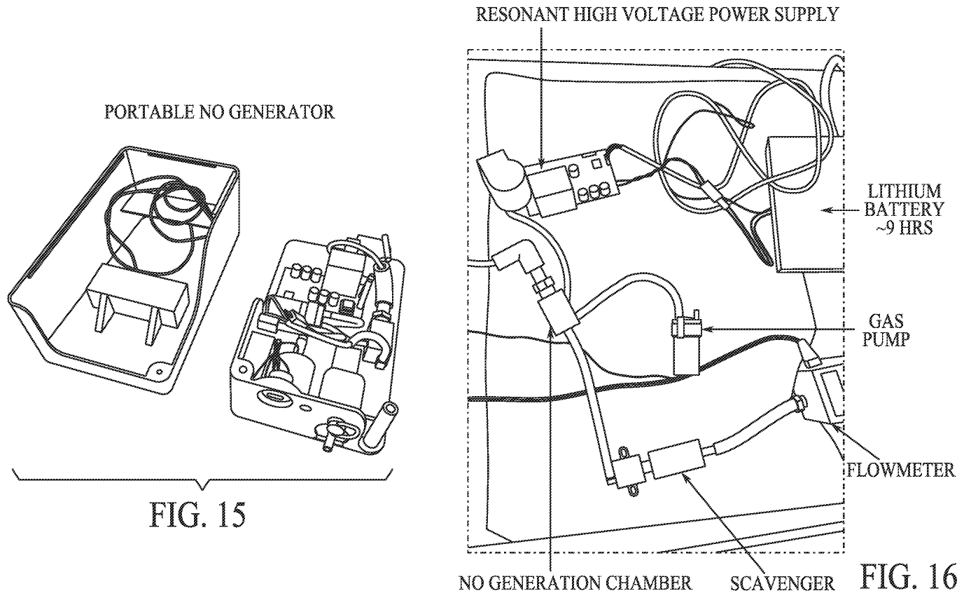

FIG. 15 shows an example of a portable NO generator.

FIG. 16 shows the parts of the portable NO generator of FIG. 15.

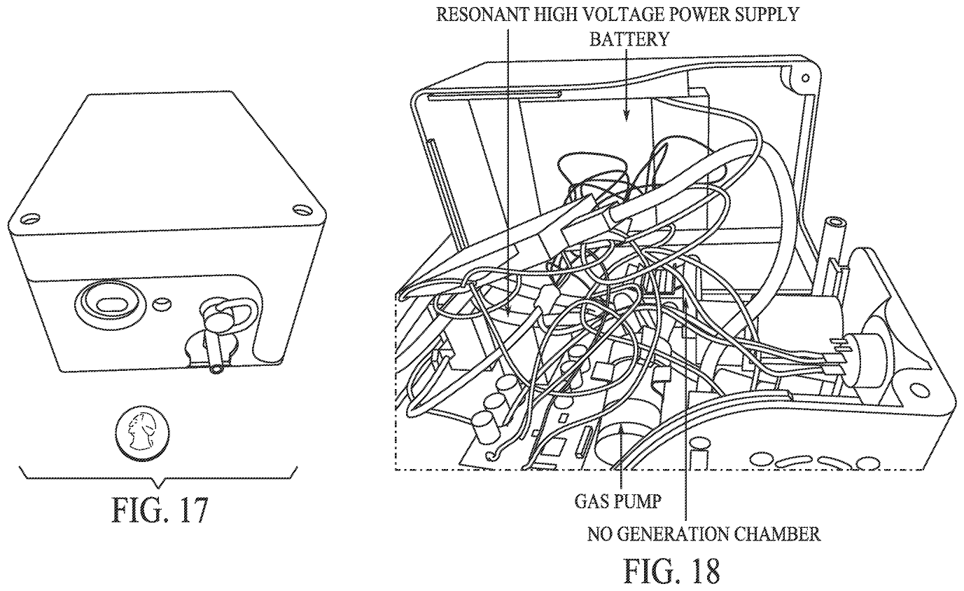

FIG. 17 shows a front isometric view of the portable NO generator of FIG. 15. The portable NO generator has a length of 21.5 cm, a width of 12.5 cm, and a height of 8.9 cm. The NO generator has a weight of 3.4 lbs.

FIG. 18 shows a side isometric view of the portable NO generator of FIG. 15, with the cover opened.

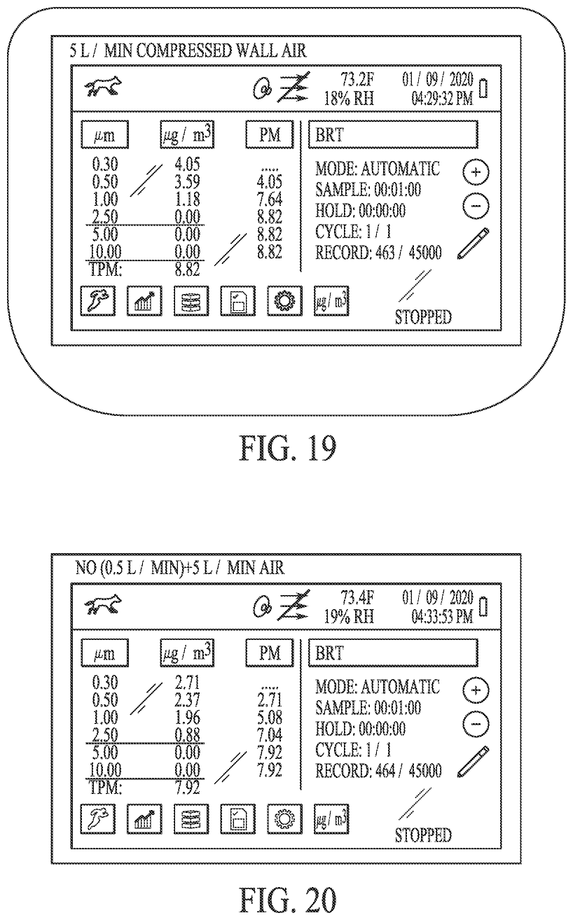

FIG. 19 shows an image of a display of a particle counter. The particle counter (Graywolf, Advanced Environment Instrumentation) detected particles within the compressed air, and the NO gas. The EPA standard is PM_{2.5}=12 μg/m³, PM₁₀=150 μg/m³.

FIG. 20 shows another image of the display of FIG. 19.

Table 3 shows the daily measurement of NO and NO₂ concentrations for 53 days of continuous NO generation.

Duty cycle = 19-22, NO is delivered at 0.5
L/min, diluted with O₂ = 5 L/min

| Time | NO | NO₂ |
|---|---|---|
| (Day) | (ppm) | (ppm) |
| Day 1 | 50.1 | 0.88 |
| (Dec. 9) | | |
| Day 2 | 51.2 | 0.91 |
| Day 3 | 50.2 | 0.86 (change a new |
| | | blue scavenger) |
| Day 4 | 52.6 | 1.13 |
| Day 5 | 51.4 | 1.21 |
| Day 6 | 52.3 | 1.32 |
| Day 7 | 53.1 | 1.44 |
| Day 8 | 50.4 | 1.36 (reduce duty |
| | | cycle to 20) |
| Day 9 | 49.8 | 0.85 (change a new |
| | | scavenger) |
| Day 10 | 50.1 | 1.10 |
| Day 11 | 50.1 | 1.47 |
| Day 12 | 52.5 | 1.10 (change a new |
| (Dec. 20) | | scavenger) |
| Day 13 | 50.2 | 1.15 |
| Day 14 | 50.3 | 1.28 |
| Day 15 | 50.9 | 1.21 (change a new |
| | | scavenger) |
| Day 16 | 50.1 | 1.18 |
| Day 17 | 49.9 | 1.25 |
| Day 18 | 50.2 | 1.10 (change a new |
| | | scavenger) |
| Day 19 | 49.8 | 1.20 |
| Day 20 | 50.3 | 1.26 |
| Day 21 | 50.5 | 1.11 (change a new |
| | | scavenger) |
| Day 22 | 50.2 | 1.15 |
| Day 23 | 49.8 | 1.20 |
| Day 24 | 50.0 | 1.25 |
| (Jan., 1, 2020) | | |
| Day 25 | 50.1 | 1.20 (change a new |
| | | scavenger) |
| Day 26 | 50.5 | 1.38 |
| Day 27 | 49.6 | 1.42 |
| Day 28 | 50.2 | 1.61 |
| Day 29 | 49.5 | 1.22 (change a new |
| | | scavenger) |
| Day 30 | 51.1 | 1.31 |
| Day 31 | 50.2 | 1.38 |
| Day 32 | 49.8 | 1.44 |
| (Jan., 9, 2020) | | |
| Day 33 | 50.1 | 1.45 |
| Day 34 | 50.6 | 1.50 |
| Day 35 | 49.5 | 1.48 |
| Day 36 | 49.8 | 1.50 |
| Day 37 | 49.3 | 1.49 Opened up to |
| (Jan., 14, 2020) | | check electrode |
| Day 38 | 50.1 | 1.10 (change a new |
| | | scavenger) |
| Day 39 | 51.2 | 1.34 |
| Day 40 | 49.6 | 1.40 |
| Day 41 | 50.2 | 1.56 |
| Day 42 | 49.2 | 1.61 |
| Day 43 | 51.1 | 1.79 |
| Day 44 | 50.3 | 1.83 |
| Day 45 | 49.8 | 1.88 |
| Day 46 | 50.4 | 1.94 |
| Day 47 | 49.7 | 1.92 |
| Day 48 | 51.2 | 1.95 |
| Day 49 | 49.6 | 2.21 |
| Day 50 | 49.2 | 2.32 |
| Day 51 | 50.1 | 0.92 (change a new |
| | | scavenger) |
| Day 52 | 51.1 | 1.21 |
| Day 53 | 49.9 | 1.32 |
| (Jan., 30, 2020) | | |

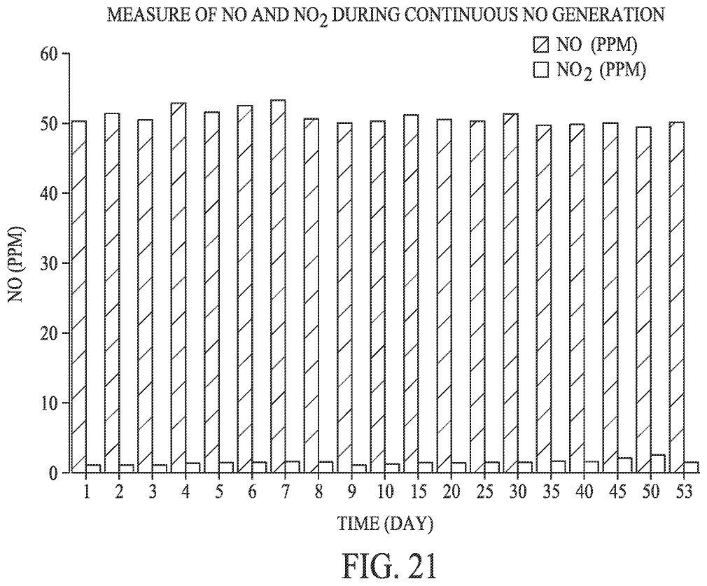

FIG. 21 shows a graph of the NO and NO₂ concentration (ppm) versus the time (days) during continuous NO generation.

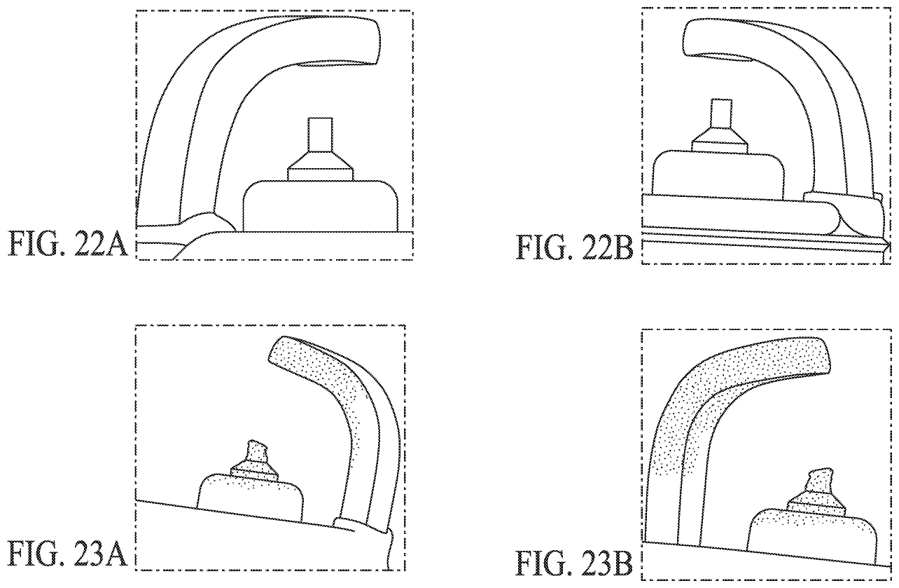

FIGS. 22A and 22B show different views of a new spark plug.

FIGS. 23A and 23 B show different views of the spark plug of FIGS. 22A and 22B after 37 days of use.

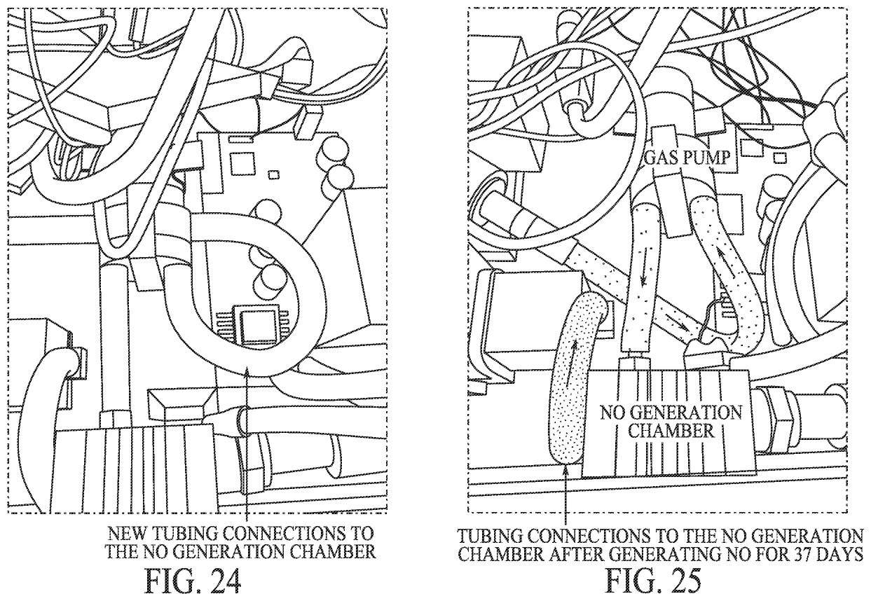

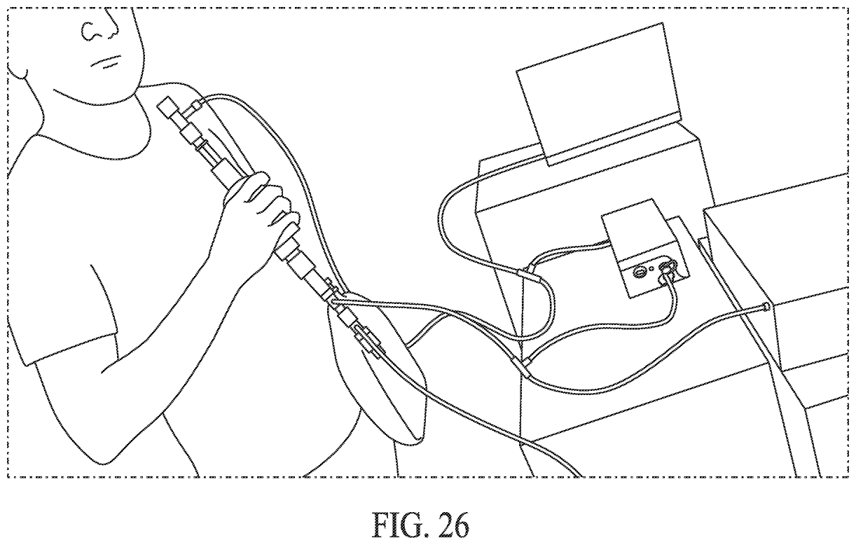

FIG. 24 shows a top view of new tubing connections to the NO generation chamber before the 37 days of use.

FIG. 25 shows another top view of the tubing connections to the NO generation chamber after the 37 days of use.

FIG. 26 shows an image of a user breathing high doses of NO generated from air by the eNO device with a snug fitting mask.

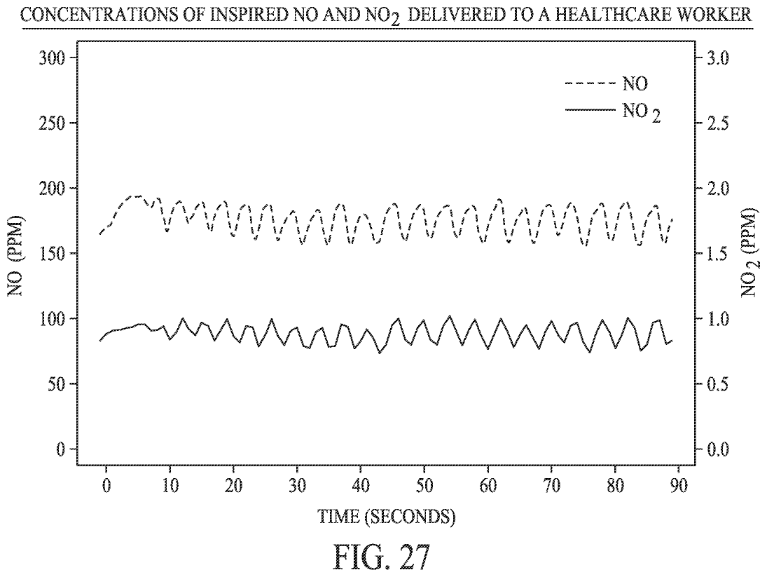

FIG. 27 shows a graph of the NO concentration (ppm) and NO₂ concentration (ppm) of inspired gas delivered to a healthcare worker over time (seconds).

In summary, the portable NO generator produces 50 ppm NO for more than 50 days, the scavenger (6.5 g CA(OH)₂) needs to be refreshed every 2-3 days to keep NO₂ levels below or at 1 ppm, and the electrodes erode during NO generation and need to be replaced every month. Regarding the in vivo tests, the portable NO generator generates high-dose of NO (e.g., 160 ppm) with the NO₂ level being below 1 ppm after a scavenger. Using a snug fitting mask, the high dose of NO can be safely delivered to a patient

### Example 3

A novel inhalation mask system to deliver high concentrations of nitric oxide gas in spontaneously breathing subjects is provided in this example. Nitric Oxide (“NO”) is administered as a gas for inhalation to induce selective pulmonary vasodilation. It is a safe therapy, with few potential risks even if administered at high concentration. No device is currently available to easily administer inhaled NO at concentrations higher than 80 parts per million (ppm) at various inspired oxygen fractions, without the need for dedicated, heavy, and costly equipment. The development of a reliable, safe, inexpensive, lightweight, and ventilator-free solution is crucial, particularly for the early treatment of non-intubated patients outside of the intensive care unit (“ICU”) and in a limited-resource scenario. To overcome such a barrier, a system for the non-invasive NO gas administration up to 250 ppm was developed using consumables and a scavenging chamber. The method has been proven safe and reliable in delivering a specified NO concentrations while limiting nitrogen dioxide levels. This example aims to provide the necessary information on how to assemble or adapt such a system for research purposes or clinical use in patients with acute respiratory syndrome coronavirus 2 (“COVID-19”) or other diseases in which NO administration might be beneficial.

NO inhalation therapy is regularly used as a life-saving treatment in several clinical settings. In addition to its pulmonary vasodilator effect, NO displays a broad antimicrobial effect against bacteria, viruses, and fungi, particularly if administered at high concentrations (>100 ppm). During the 2003 Severe Acute Respiratory Syndrome (“SARS”) outbreak, NO showed potent antiviral activity in vitro and demonstrated therapeutic efficacy in patients infected with the SARS-Coronavirus (“SARSCoV”). The 2003 strain is structurally similar to SARSCov-2, the pathogen responsible for the current Coronavirus Disease-2019 (“COVID-19”) pandemic.

The development of an effective and safe treatment for COVID-19 is a priority for the healthcare and scientific communities. To investigate the administration of NO gas at doses >80 ppm in non-intubated patients and volunteering healthcare workers, the need to develop a safe and reliable non-invasive system became apparent. This technique aims to administer high NO concentrations at different fractions of inspired oxygen (“FiO₂”) to spontaneously breathing subjects. This proposed system is currently in use to conduct a series of randomized controlled trials to study the following effects of high concentrations of NO gas. First, the effect of 160 ppm NO gas is being studied in non-intubated subjects with mild-moderate COVID-19, admitted either in the Emergency Department or as inpatients. Second, the role of high-dose NO is being examined to prevent SARSCoV-2 infection and the development of COVID-19 symptoms in healthcare providers routinely exposed to SARS-CoV-2-positive patients.

This device can be assembled with standard consumables used for respiratory therapy. The proposed apparatus is designed to non-invasively deliver a mixture of NO gas, medical air, and oxygen (“O₂”). Nitrogen dioxide (“NO₂”) inhalation is minimized to reduce the risk of airway toxicity. The current NO₂ safety threshold set by the American Conference of Governmental Industrial Hygienists is 3 ppm over an 8 hour time-weighted average, and 5 ppm is the short-term exposure limit. Conversely, the National Institute for Occupational Safety and Health recommends 1 ppm as a short-term limit of exposure. Given the increasing interest in high-dose NO gas therapy, the present report provides the necessary description of this novel device. It explains how to assemble its components to deliver a high concentration of NO for research (or other) purposes.

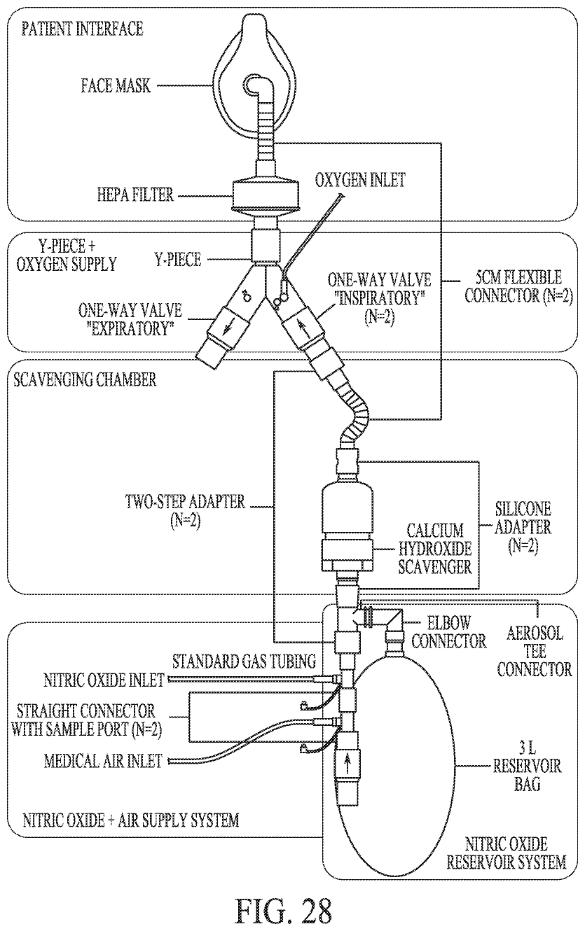

FIG. 28 shows a graphic representation of the delivery device. The single components are indicated in the figure, as named in his example. The system comprises four major parts: the patient interface; Y-piece and oxygen supply; scavenging chamber; and the NO reservoir system and NO and medical air supply system.

Building the patient interface. Take a snug-fitting, standard, non-invasive ventilation face mask of the appropriate size for the subject. Connect the mask's built-in elbow port to a high-efficiency particulate air (highly hydrophobic bacterial/viral filter, HEPA class 13) filter through the 22 mm outer diameter (O.D.)/15 mm inner diameter (I.D.) connector. Optionally, to facilitate the subject's movement and reduce the risk of disconnection, add a 15 mm O.D.×22 mm O.D./15 mm I.D. (length 5 cm-6.5 cm) flexible patient connector for an endotracheal or tracheostomy tube between the mask interface and the HEPA filter. Every effort should be made to avoid leakage of the mask interface. The “patient end” of the device could also consist of a mouthpiece, but a nose clip should be added in such a configuration.

Building the Y-piece and preparation of the O₂ supply. Take a 22 mm to 22 mm and 15 F Y-piece connector with 7.6 mm ports. Create the circuit's expiratory and inspiratory limbs on the two distal ends of the Y-piece through two opposite-sense, low-resistance, 22 mm male/female, one-way valves. Expiratory limb: on one end of the Y-piece, place the one-way valve connector allowing a proximal to distal flow only (arrow pointing downward in FIG. 28). Inspiratory limb: on the other end of the Y-piece, connect a one-way valve allowing a distal to proximal flow only (arrow pointing upward in FIG. 28). Connect the proximal end of the Y-piece (e.g., including the one-way valve) to the HEPA filter. Using standard, kink-resistant, vinyl gas tubing with universal adaptors at both ends, connect the O₂ source to the Y-piece's inspiratory limb (e.g., downstream of the one-way valve). Choose tubing of the appropriate length considering the distance between the patient and the source of the gas. In some cases, it is important that the Y-piece connector have a sampling port on the inspiratory limb. If not, an additional straight connector with a sampling port can be used to supply O₂ for inspiration for the patient.

Building and attaching the scavenging chamber. Connect a 22 mm×22 mm silicon rubber, flexible connector adapter to the proximal end of a scavenger chamber (internal diameter=60 mm, internal length=53 mm, volume=150 mL) containing 100 g of calcium hydroxide (Ca(OH)₂). Attach a 15 mm outer diameter (“OD”)×22 mm OD/15 mm inner diameter (“ID”), 5 cm-6.5 cm, flexible, corrugated tube to the silicon rubber adapter. Connect another 22 mm×22 mm silicon rubber, flexible connector adapter to the distal end of the scavenger. Add the scavenging chamber and tubing assembly to the Y piece's inspiratory limb using a 15 mm-22 mm two-step adapter.

Building and attaching the NO reservoir system. Assemble a 3-L latex-free breathing reservoir bag and a 90° ventilator elbow connector without ports (22 mm ID×22 mm). Connect the other end of the elbow to the central opening of the aerosol T-piece (horizontal ports 22 mm OD, vertical port 11 mm ID/22 mm OD). Attach the T-piece to the scavenging chamber's distal end by advancing it until it fits the silicon rubber connector tightly.

Building the NO and medical air supply system. Build the NO/air gas supply system by attaching two consecutive 15 mm OD×15 mm ID./22 mm OD connectors with 7.6 mm sampling ports and flip-top caps. Once the caps are removed, the sampling accesses can function as gas inlet ports. At the distal end of the NO/air supply system, attach another one-way inspiratory valve (arrow pointing upwards towards the bottom of FIG. 28). At the proximal end of the NO/air supply system, connect a 15/22 mm two-step adapter. Connect the proximal two-step adapter to the remaining free inlet of the green T-piece from the NO reservoir system.

Attach the air and NO gas flow lines by using standard, kink-resistant, star-lumen vinyl oxygen gas tubing for the following steps. Connect medical air to the most distal gas inlet port. Connect NO gas from an 800 ppm medical-grade NO tank (size AQ aluminum cylinders containing 2239 L of 800 ppm of NO gas at standard temperature and pressure, balanced with nitrogen; delivered volume 2197 L) to the next port downstream of the port that receives medical air. It is importing that tubing be of appropriate length to reach the gases' sources comfortably. Different tanks or generators of NO can be used as sources of gas.

Use in spontaneously breathing subjects. Set the air, O₂, and NO gas flow according to the desired FiO₂ and NO concentration. The recommended flow rates for administering NO at 80, 160, or 250 ppm are listed in Table 4 (applicable to 800 ppm cylinders only). Position the tight-fitting mask on the patient's face, similar to a non-invasive ventilation interface setup. Start the inhalation session for the desired duration.

TABLE 4
Setup of NO, O₂, and air gas flows.

| Target NO | FiO₂ | Flow setup (L/min) | | | Measured NO₂ |
|---|---|---|---|---|---|
| (ppm) | (%) | NO | O₂ | Air | (ppm) |
| 80 | 21 | 1.67 | 1.28 | 15 | 0.32 |
| | 30 | 1.89 | 3.28 | 15 | 0.32 |
| | 40 | 2.21 | 7.24 | 15 | 0.37 |
| 160 | 21 | 3.87 | 1.78 | 15 | 0.81 |
| | 30 | 4.38 | 4.31 | 15 | 1.05 |
| | 40 | 5.38 | 9.59 | 15 | 1.2 |
| 250 | 21 | 6.99 | 2.1 | 15 | 1.57 |
| | 30 | 9.1 | 7.3 | 15 | 2.35 |
| | 40 | 11.91 | 17.4 | 15 | 2.61 |

Regarding Table 4, gas flows to deliver target NO concentrations at varying FiO₂, as measured with a lung simulator in a bench experiment. NO, and O₂ flow (in L/min) were set to obtain target NO inspiratory concentration (80, 160, and 250 ppm) at the desired FiO₂ (21%, 30%, 40%). A constant medical air flow rate (15 L/min) was used in every setting. A commonly available 800 ppm NO cylinder balanced with nitrogen was used.

Representative Results. A 33-year-old respiratory therapist working at the intensive care unit (“ICU”) at Massachusetts General Hospital (“MGH”) during the surge of ICU admission for COVID-19 volunteered to receive NO as part of the trial involving healthcare workers. The trial tested the efficacy of 160 ppm of NO as a virucidal agent, thereby preventing disease occurrence in lungs at risk for viral contamination. The first session of the inhalation prophylaxis was administered before starting a shift through the described device for 15 min. For research purposes, concentrations of inhaled NO, NO₂, and O₂ were continuously measured. NO gas was administered at 3.5 L/min from an 800 ppm gas tank and mixed with air at a flow rate of 15 L/min and an O₂ flow rate of 1 L/min to maintain a FiO₂ at 21%.

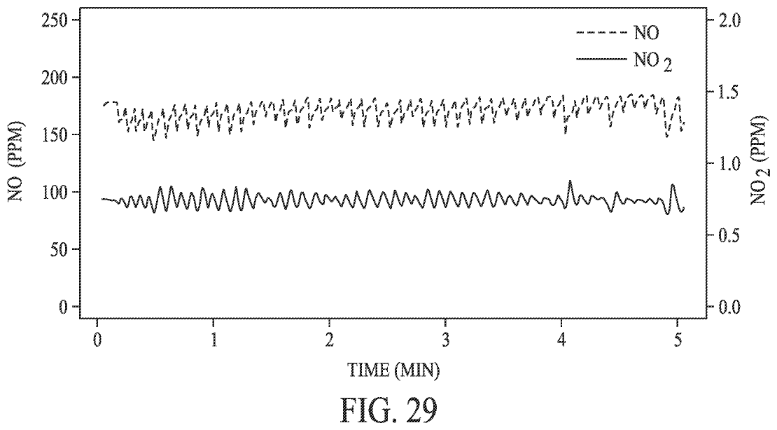

The resulting NO concentration was 160 ppm at a total gas flow rate of 19.5 L/min, measured by three standard 15 L/min flowmeters. Oxygen saturation (“SpO₂”), methemoglobin (“MetHb”), and heart rate were continuously monitored. SpO₂ remained stable at around 97%. MetHb peaked at 2.3% during NO administration before rapidly returning to the baseline value upon suspension of the gas. The subject did not experience any side effects during or after the session. The NO concentration remained stable throughout the whole period of inhalation. NO₂ peaked at 0.77 ppm and was therefore safely below the recommended toxicity threshold. A representative portion of the recorded tracings of NO and NO₂ signals is depicted in FIG. 29.

Given the increasing interest in NO gas therapy for non-intubated patients, including those with COVID-19, this example describes a device and how to assemble its components to deliver NO at concentrations as high as 250 ppm. The proposed system is built out of inexpensive consumables and safely delivers a reproducible concentration of NO gas in spontaneously breathing patients. The ease of assembly and use, together with the safety data published elsewhere, makes this system an ideal embodiment to administer a high NO gas concentration at varying FiO₂ in non-intubated patients. The methodology described herein is currently in use at MGH to investigate the effect of high concentrations of NO to treat, or prevent, COVID-19. The method can be adjusted based on the local availability of specific consumables, which may differ in brand and size from those described here. Nevertheless, a few critical steps of the protocol should be followed. The sequence of each gas supply line, the reservoir bag, and the unidirectional valves should not be altered for any reason. A high-efficiency particulate absorbing (“HEPA”) filter should also be present, particularly in case of any risk of infected bio-aerosol dispersion to the environment. Air leaks might impact the delivery of appropriate NO concentrations. Care should be exercised to use appropriately positioned and sized face masks and to avoid disconnection of the system. The availability of a scavenger chamber with at least the reported amount (100 g) of Ca(OH)₂ can be important to prevent the accumulation of NO₂ and avoid nitric acid formation upon reaction with water in the lungs. The Ca(OH)₂ scavenger is designed to undergo a chemical dye reaction upon consumption, functioning as an indicator of its residual absorbent properties. To ensure the efficiency of the scavenger in reducing NO₂ levels, the component should be changed when two-thirds of the canister have changed color.

Bench tests showed that NO₂ remained below 1 ppm for the first 60 min and never exceeded 1.3 ppm even after 5 h of exposure to 160 ppm NO. Sessions longer than five hours will likely require the scavenger to be changed. In case a cylinder is used as a source of NO gas, attention must be paid to the native NO concentration in the tank, as reported by the manufacturer. The NO, air, and O₂ flow settings for a standard NO high-pressure cylinder are reported (see Table 4). The use of cylinders with different gas concentrations, or alternative NO generating devices, would impact the flow settings necessary to deliver gas mixtures with the desired NO and O₂ concentrations. NO is diluted in nitrogen as a balance gas in most high-pressure cylinders. The higher the NO concentration, the lower the net FiO₂ is administered to the patient if no supplemental O₂ is added to the mixture. This interplay between NO concentration and FiO₂ must be considered, especially when NO is administered to an already hypoxic patient, or while assessing the efficacy of NO in terms of oxygenation improvement. The resulting SpO₂ increase might be blunted if FiO₂ is not maintained constant during NO administration. Importantly, if no supplemental O₂ is administered, a hypoxic mixture can potentially be generated by mixing high-dose NO and air.

NO has a very favorable safety profile. The molecule's very short half-life further limits the few potential adverse effects. Methemoglobinemia is the most important threat, particularly in the setting of prolonged high-dose exposure. Thus, MetHb levels should always be monitored closely. MetHb is formed in the blood upon breathing NO by the oxidation of iron present in circulating hemoglobin. Measurements can be obtained through rapid blood testing or non-invasively through methemoglobin saturation (“SpMet”) percentage monitoring. Levels up to 10% are usually well-tolerated in healthy subjects. Hemodynamic deterioration can rarely occur following NO inhalation. Rebound pulmonary hypertension is another possible risk if the prolonged administration of NO is abruptly interrupted. The device can be modified to sample gas concentrations if needed. A NO/NO₂ sampling access (15 mm straight connector with port) can be placed at the inspiratory limb, before the Y-piece. In that case, to safely add O₂ to the admixture, an additional 15 mm straight connector with a port can be placed upstream and used as an oxygen inlet. However, monitoring the inspired gas concentrations of NO and NO₂ is difficult because of technical difficulties and the need for dedicated equipment to measure ppm levels of these gases at the bedside. Despite using the same tank, slight variations in the administered concentration might occur, compared to those reported in Table 1, based on the patient's minute ventilation. Additionally, standard gas rotameters (0-15 L/min with a stainless steel ball float) do not allow increments smaller than 0.5 L. The availability of high-precision digital flowmeters, similar to those for the setup shown in Table 4, would increase the precision of the dose being administered.

Although convincingly performing in bench experiments and testing on volunteers and patients, to date, data are based on experience limited to a single center. Operators should engage in the use of this system and the administration of high-dose NO only if already experienced in the use of NO gas therapy to treat critically ill patients. Depending on the local institutional policy and agreements in force, tanks or other NO gas sources might be challenging to obtain and use as freely adjustable gas sources, outside of the limitations imposed by the delivery devices currently available on the market. NO is an endogenously produced vasodilator. Its administration as a gas therapy is currently approved by the U.S. Food and Drug Administration “for the treatment of term and near-term neonates with hypoxic respiratory failure associated with clinical or echocardiographic evidence of pulmonary hypertension”. However, NO is also routinely used in adults for pulmonary vasoreactivity testing and as rescue therapy in hypoxemic critically ill patients with or without pulmonary hypertension. The safety and tolerance of a high concentration (160 ppm) of NO have been consistently reported in studies addressing the drug's virucidal, bactericidal, and fungicidal effects. To administer high-dose NO for research purposes, internal review board (“IRB”) approval was sought and obtained. To date, the administration of inhaled NO mainly relies on gas tanks and associated bulky machinery. Tank-based delivery devices are commonly designed to administer NO gas concentrations up to 80 ppm. NO inhalation can be continuous or synchronized with the patient's inspiration. Measuring NO, NO₂ and O₂ concentrations through an electrochemical sensor cell is possible. Such expensive devices may offer technical and safety advantages compared to the construction described in this example. However, they are expensive and rarely present in more than a few units, being generally used within selected ICUs in intubated patients. As a result, the availability of NO therapy for patients outside the ICU is very limited, even at large institutions. Furthermore, the majority of currently marketed devices do not allow the off-label administration of concentrations higher than 80 ppm. Not surprisingly, by means of currently available devices, it is virtually impossible to administer NO at high concentrations on a large scale in a limited-resource setting, such as that mandated by a surge of patients and a shortage of medical supplies. Under such circumstances, the need for a simple and inexpensive, yet safe, device for the administration of this potentially beneficial therapy is critical. This system might be implemented in the future by more investigators and clinicians to safely and reliably administer NO in a reproducible way in COVID-19 and other disease states for which NO properties might be beneficial. In the described methodology, the source of NO is usually a standard gas tank. Other NO sources can be adapted to be used with this delivery system, including tankless devices and generators.

### Example 4

There is an increasing interest in safely delivering a high dose of inhaled nitric oxide (“NO”) as an antimicrobial and antiviral therapeutic for spontaneously breathing patients. An NO delivery system is described in this example. A gas delivery system was developed that utilizes respiratory circuit connectors, a reservoir bag, and a scavenging chamber containing calcium hydroxide. The performance of the system was tested using a mechanical lung, assessing the NO concentration delivered at varying inspiratory flows. Safety was assessed in vitro and in vivo by measuring nitrogen dioxide (“NO₂”) levels in the delivered NO gas. Lastly, the inspired and expired NO and NO₂ of this system was measured in 5 healthy subjects during a 15-min administration of high dose NO (160 parts-per-million, ppm) using the delivery system. The system demonstrated stable delivery of prescribed NO levels at various inspiratory flow rates (0-50 L/min). The reservoir bag and a high flow of entering air minimized the oscillation of NO concentrations during inspiration on average of 4.6 ppm for each 10 L/min increment in lung inspiratory flow. The calcium hydroxide scavenger reduced the inhaled NO₂ concentration on average of 0.9 ppm. It should be noted that other metal hydroxides such as sodium hydroxide, lithium hydroxide and potassium hydroxide can also be used. 49 NO administrations of 160 ppm were performed in 5 subjects. The average concentration of inspired NO was 164.8±10.74 ppm, with inspired NO₂ levels of 0.7±0.13 ppm. The subjects did not experience any adverse events; transcutaneous methemoglobin concentrations increased from 1.05±0.58 to 2.26±0.47. The system that was developed to administer high-dose NO for inhalation is easy to build, reliable, was well tolerated in healthy subjects.

Nitric oxide (“NO”) is a therapeutic gas that was approved by the US Food and Drug Administration (“FDA”) in 1999 for the treatment of “term and nearterm (x>34 weeks) neonates with hypoxic respiratory failure associated with clinical or echocardiographic evidence of pulmonary hypertension where it improves oxygenation and reduces the need for extracorporeal membrane oxygenation”. In addition to its pulmonary vasodilator effect, NO produces broad antimicrobial activity on bacteria and viruses such as SARS CoV, the virus responsible for the SARS epidemic in 2003.

In spontaneously breathing patients, NO gas is traditionally delivered through a mechanical ventilator or through a high flow nasal cannula (“HFNC”) system. Nitric oxide is blended with medical air and oxygen in the ventilator and may be delivered to the patient through a snug fitting facemask. Despite this approach's ability to administer NO gas, widespread adoption is challenged by the lack of a safe delivery system. In addition, delivering NO via HFNC or ventilator-driven respiratory systems potentially aerosolizes droplets with virus, which raises further concerns for safety. Two major patient safety aspects of administering high-dose NO are the generation of nitrogen dioxide (NO₂) and methemoglobin (“MetHb”). Nitrogen dioxide is formed by the reaction between NO and oxygen, and when combined with water in the airways, NO₂ forms nitric acid, leading to a caustic burn of the airways. When inhaling NO, methemoglobin is generated by oxidation of the iron contained in circulating hemoglobin. Methemoglobin cannot bind oxygen, so levels must be closely monitored in all subjects receiving NO (particularly high doses). Methemoglobin levels of 10%, or less, are well tolerated in a healthy subject. After cessation of NO treatment, intracellular methemoglobin reductase rapidly reduces RBC MetHb levels. An in-hospital system that is simple, inexpensive, and capable of delivering a constant and predictable concentration of NO over time, while minimizing NO₂, without generating aerosolized particles is needed to allow use of high-dose NO outside the intensive care unit (“ICU”).

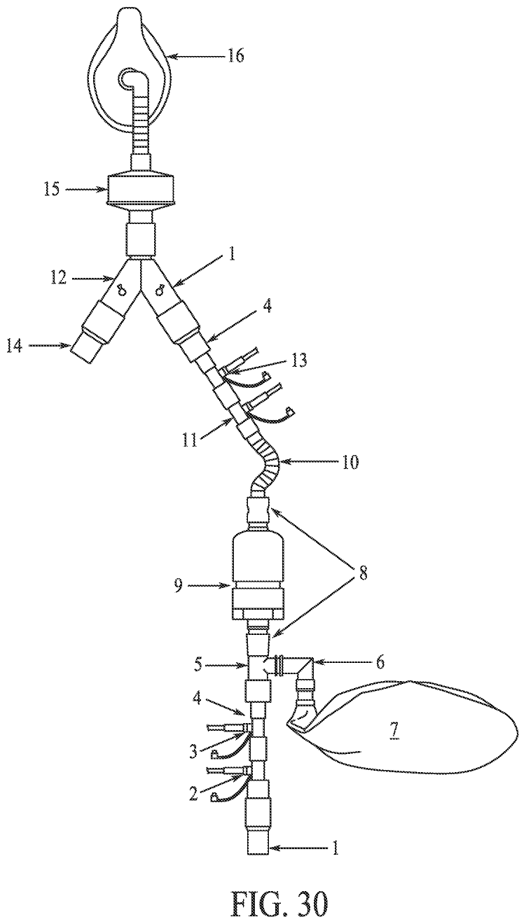

In this example, a breathing system capable of delivery high concentrations of NO was developed and studied. The performance and safety of the device was evaluated both in vitro and in healthy adults by accurately sampling and measuring NO and NO₂ concentrations in the inhaled and exhaled breath. FIG. 30 illustrates the breathing system with the components being labeled as follows: 1. Inspiratory one-way valve; 2. Gas connector for medical air; 3. Gas connector for NO; 4. Two-step adapter; 5. T Adaptor; 6. Elbow Adaptor; 7. Reservoir bag (3 L); 8. Silicone adapter; 9. NO₂ Scavenger; 10. Flex Connector; 11. Gas connector for O₂; 12. Y-piece; 13. Gas sample port for NO and NO₂ analyzers; 14. Expiratory one-way valve; 15. HEPA filter; 16. Snug fitting full face mask.

The system incorporates respiratory circuit connectors (see FIG. 30). The distal portion of the inspiratory limb begins with a one-way valve (Hudson RCI, Wayne, Pa., USA) and two gas inlet connectors (Hudson RCI, Wayne, Pa., USA), which inject medical air and NO gas, respectively. A T-connector joins a 3 L bag to the inspiratory limb. The bag serves as an NO reservoir to stabilize the NO concentration throughout the inspiratory phase. A scavenger (internal diameter=60 mm, internal length=53 mm, volume=150 mL) containing 100 g of calcium hydroxide (Spherasorb™, Intersurgical Ltd, Berkshire, UK) absorbs the NO₂ generated in the gas mixture. A flexible connector was inserted to accommodate patient movement and positioning. Two gas inlet connectors act as oxygen inlet and NO/NO₂ sampling line, respectively. A second inspiratory one-way valve was placed after the set reservoir/scavenger to avoid additional gas mixing due to expired backflow. This system was created for the treatment of subjects with coronavirus disease 2019 (“COVID-19”), and a high-efficiency particulate air (“HEPA”) filter was connected between the Y-piece and the patient interface (full face mask or mouthpiece) to remove any aerosolized virus. Active humidification was not added, and relative humidity ratio was not tested, as the device, here described, has been built for delivering intermittent, short periods of high dose nitric oxide.

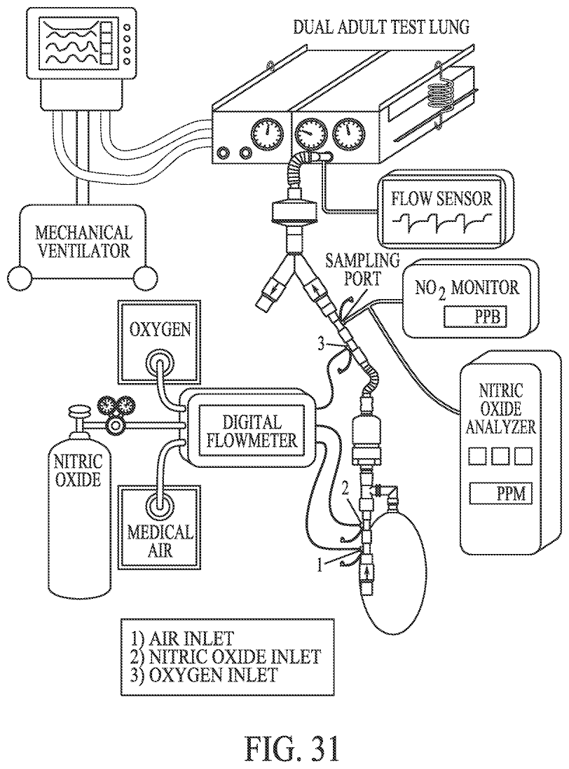

The NO delivery system performance was tested using a bench testing lung (Dual Adult Test Lung, Michigan Instruments, Michigan, USA) (see FIG. 31) and a mechanical ventilator to simulate an inspiratory effort (Hamilton G5, Hamilton Medical AG, Bonaduz, Switzerland). The ventilator was connected to the right lung, which acts as the “diaphragm” to lift the left lung by a coupling clip. An inspiratory sinusoid flow waveform was produced by the ventilator during a volume controlled ventilation mode. The iNO system was connected to the left lung (the “breathing” lung), which was set with a compliance of 0.05 L/cmH₂O. No airway resistor was added. A digital flowmeter (Mallinckrodt Puritan-Bennett PTS 2000) was used to measure the air, O₂, and NO gas flow rates. The inspired oxygen fraction was assessed with an oxygen analyzer (MiniOX® 1, Ohio Medical Corporation®, 1111 Lakeside Drive, Gurnee, Ill. 60031 USA).

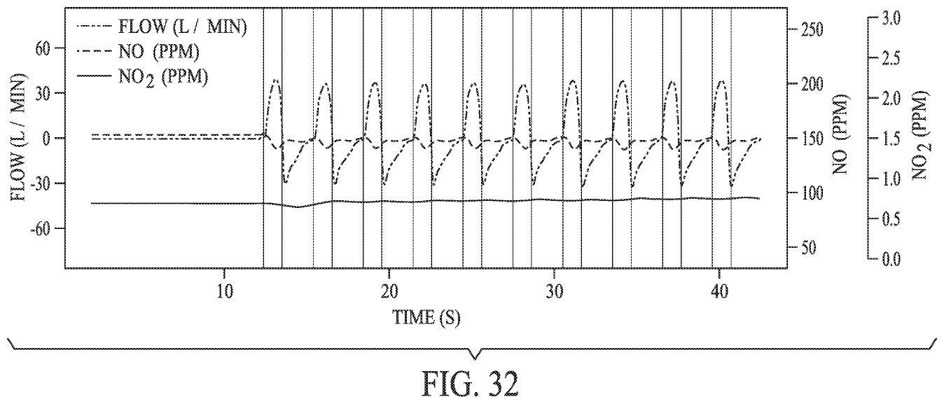

The NO concentration was measured by an NO analyzer (Sievers 280i Nitric Oxide Analyzer, GE Analytical Instruments, Boulder, Colo.) connected to the inspiratory limb via a sampling line proximal to the Y-connector. NO₂ levels were simultaneously evaluated by the Cavity Attenuated Phase Shift (“CAPS”) NO₂ monitor (Aerodyne Research Inc, Billerica, Mass.) using the same sampling port and line. NO and NO₂ concentrations were measured during the inspiratory phase (see FIG. 32). Additionally, we measured NO and NO₂ concentration using an electrochemical gas sensor from a commercially available NO delivery system (iNOmax DSIR® Plus, Mallinckrodt Pharmaceuticals, Bedminster, N.J., USA). The NO tank was provided at either 857 ppm (150 A, Airgas, Radnor Township, Pennsylvania, content=4089 L at standard temperature and pressure (“STP”)) or 800 ppm (Noxivent, size AQ aluminum cylinders Praxair Shimersville Road Bethlehem Pa., content=2239 L at STP). The duration of a 2239 L tank at STP ranged between 3.1 and 37.3 h when NO was delivered at 50 ppm or 250 ppm, respectively. One should note, however, that the delivery system we described here is independent from the tank of NO employed. By introducing a standard gas connector, an operator could use our delivery system with any desirable NO source.

To reduce the fluctuations of delivered NO, the efficacy of adding a reservoir bag was examined (e.g., for stabilizing the concentration of the inspired NO). The experimental setup involved a respiratory rate (“RR”) of 15 breaths/min; a tidal volume (“TV”) of 0.25, 0.5, 0.75 and 1 L; an inspiratory time of 1 s, and a sinusoidal flow wave. During this test the calcium hydroxide scavenger was incorporated into the system. The average inspiratory flow required to archive the set TV was used as an independent variable in the analysis. Nitric oxide concentration was measured over 2 min during the inspiratory phase of each set of tidal volume, with and without the reservoir bag. Three NO concentrations were used: 50, 150 and 250 ppm. FiO₂ was set at 0.21. Similarly, the inspired concentration of NO₂ was evaluated, using the same experimental settings, with or without the 3 L reservoir bag.

The effect of various levels of air flow on NO concentration during ventilation was examined. The inspiratory NO concentrations were examined at 5, 10, and 15 L/min of air flow. At every level of air flow, before starting ventilation, NO gas flow was set to achieve a static concentration of 180 ppm NO in the inspiratory limb of the circuit. Ventilator settings included a respiratory rate of 20 bpm, a tidal volume of 0.5 L, an inspiratory time of 1 s and sinusoidal flow wave. The performance of the system was tested to find the NO and oxygen flow required to obtain the desired concentration of inspired NO. Mechanical ventilation was set with a tidal volume 0.5 L, a respiratory rate of 20 bpm, an inspiratory time of 1 s and a sinusoidal flow wave. Three different target NO concentrations were tested: 50, 150, and 250 ppm at different FiO₂: 0.21, 0.30, and 0.40. NO₂ levels were also measured.

To assess the efficacy of the calcium hydroxide scavenger in reducing the inspiratory levels of NO₂, the same mechanical ventilator settings were used as above, including the range of target NO concentrations (50, 150, and 250 ppm), two different levels of FiO₂ (0.21 and 0.40), and measured NO₂ levels in the inspiratory limb with and without the scavenger. High-dose NO was administered using the system to healthy adult subjects as part of a randomized controlled trial conducted in the center. Each administration lasted for 15 min. FiO₂, NO and NO₂ concentrations were monitored in the inspiratory limb of the circuit. Peripheral oxygen saturation (SpO₂) and methemoglobin (MetHb) were continuously and non-invasively monitored with a pulse co-oximeter (Masimo rainbow SET, Irvine, Calif. 92618).

Additionally, the exhaled concentration of NO₂ was evaluated in one healthy subject. One hundred fifty (150) ppm NO was administered using the previously described system. To monitor the expiratory NO₂ concentration a sampling line was placed between the mouthpiece and the HEPA filter (see FIG. 30). Since the continuous gas flow from the inspiratory limb of the circuit can interfere with the measure, a 3-way stopcock (2100 series, Hans Rudolph INC. Shawnee, Kans., USA) was positioned before the Y connector. The closure of the stopcock at the beginning of exhalation stopped the washout effect of fresh gas coming from the inhalation arm of the circuit, allowing a sampling of exhaled gas only.

Linear mixed models were used to analyze the effect of the reservoir and the scavenger on the system's performance. Statistical significance was assumed at a two-tailed P value p<0.05. The statistical analyses were performed in R (R Core Team (2016). R: A language and environment for statistical computing. R Foundation for Statistical Computing, Vienna, Austria). Variables were expressed as mean and standard deviation (SD).

The total inspiratory resistance, from the inspiratory one-way valve to the HEPA filter, was on average 8.6 cmH₂O/L/s. The total expiratory resistance, measured from the HEPA filter to expiratory one-way valve, was on average 7.2 cmH₂O/L/s.

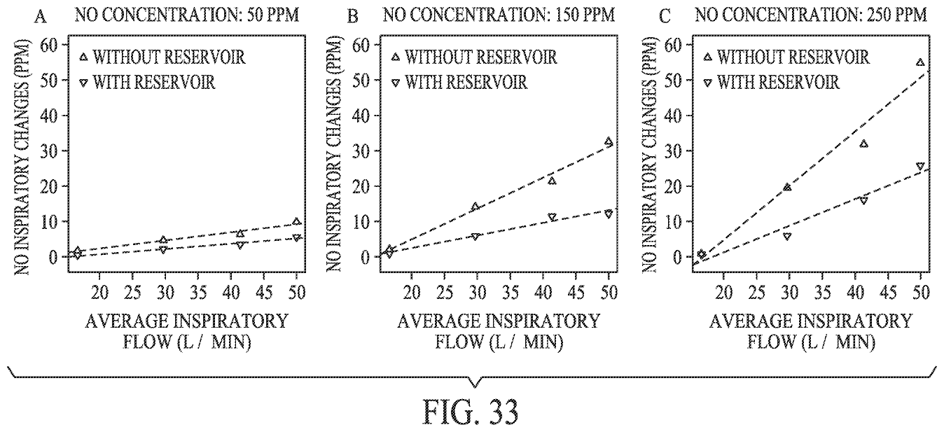

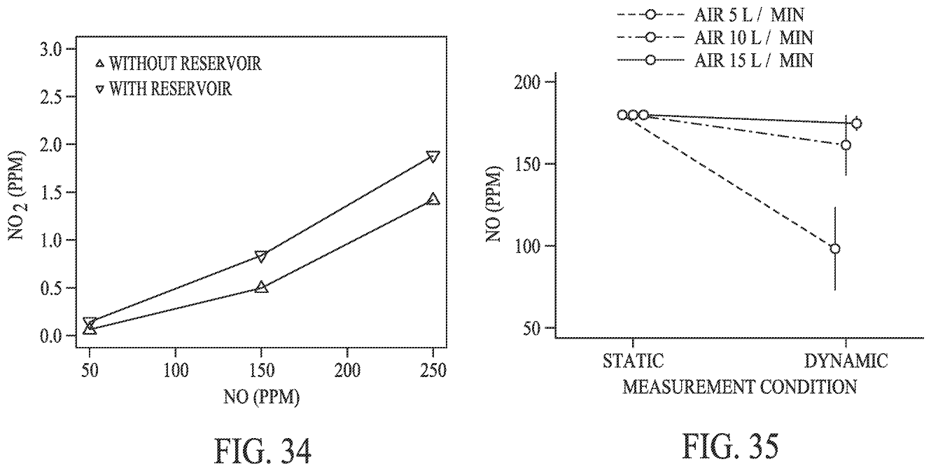

For NO concentrations of 50, 150, and 250 ppm, the reservoir bag decreased the inspiratory NO fluctuations, respectively, by 0.79 ppm (95% CI −1.7, 0.2; p″0.09), 5.2 ppm (95% CI −7.6, !2.6; p″0.004), and 7.8 ppm (95% CI −14.3, !1.3; p″0.02) for each 10 L/min increment in the average lung inspiratory flow (see FIG. 33). Nitric oxide concentrations were 50 ppm for panel A of FIG. 33, 150 ppm for panel B of FIG. 33, and 250 ppm for panel C of FIG. 33. The accumulation of NO and O₂ in the reservoir bag produced an increase of inspiratory NO₂ on average of 0.29 ppm (95% CI 0.14, 0.43; p<0.001) (see FIG. 34). The NO₂ levels were kept below 2 ppm in all NO concentrations with the reservoir bag. These data suggest that adding a reservoir bag (3 L) efficiently reduces the fluctuations of delivered NO and keeps NO₂ concentrations below the recommended level.

At a low air flow of 5 L/min, a marked reduction of NO concentration was measured in the inspiratory limb of the circuit: from 180 ppm to 96 ppm with 51 ppm of variation during the inspiratory phase. At 15 L/min, the concentration during ventilation was stable and decreasing only about 8 ppm from 180 ppm to 169 ppm (see FIG. 35). The dots of FIG. 35 represent the average inspiratory concentration, and error bars represent the intra-tidal swing of NO concentration during the inspiration phase. These findings indicate that maintaining air flow at 15 L/min reduces the effect of ventilation on the variation of the NO concentration in the respiratory limb.

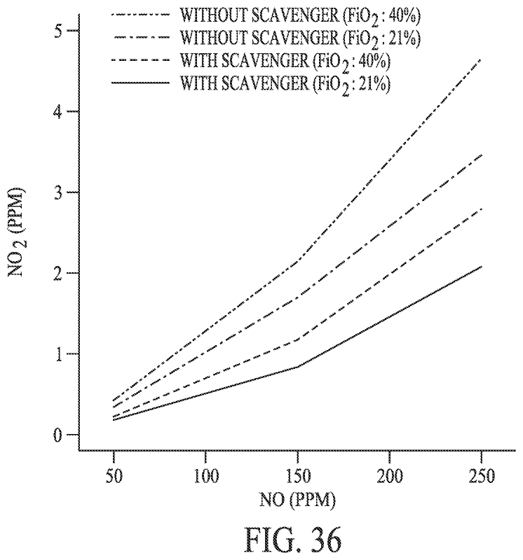

The scavenger positioned in the inspiratory limb of the circuit reduced the inhaled NO₂ concentration to an average of 0.9 ppm (95% CI −1.58, −0.22; p<0.01) (see FIG. 36). At 150 ppm of inhaled NO, the NO₂ concentration was maintained below 1.2 ppm with the FiO₂ being from 0.21 to 0.40. The data suggest that the scavenger can efficiently reduce NO₂ in the circuit for NO delivery.

The delivered NO concentrations varied depending on the flows of NO and O₂. Three concentrations of NO (50, 145, and 245 ppm) targeted for delivery were tested. First, to obtain a concentration of 50 ppm NO (48.5 ppm measured with the chemiluminescence method, 44 ppm using the electrochemical gas phase sensor), the flows of NO ranged from 1.0 to 1.4 L/min for FiO₂ levels of 0.21 and 0.41, respectively, using the 800 ppm NO/N₂ tank. Using the 857 ppm tank, NO flows were set from 0.9 to 1.2 L/min for FiO₂ levels of 0.21 and 0.41, respectively. Second, to obtain an NO concentration of 145 ppm (143 ppm measured with the chemiluminescence method, 130 ppm using the electrochemical gas phase sensor), the required NO flow was at 3.5 and 5.8 L/min of O₂ for FiO₂ levels of 0.21 and 0.41, respectively, using the 800 ppm tank. Using the 857 ppm tank, NO flows was at 3.1 L/min, and 4.5 L/min for FiO₂ levels of 0.21 and 0.41, respectively. Finally, to obtain an NO concentration of 245 ppm, the required NO flow was at 7.0 and 11.9 L/min for FiO₂ levels of 0.21 and 0.41, respectively, using the 800 ppm tank. Using the 857 ppm tank, NO flow was at 6.2 and 10.1 L/min for FiO₂ levels of 0.21 and 0.41, respectively. NO₂ concentration was measured by Cavity Attenuated Phase Shift (CAPS) NO₂ monitor and by a commonly used electrochemical gas phase sensors in hospital settings. In-vitro studies were performed by using 800 ppm of NO/N₂ tanks. Same studies, then, were repeated with 857 ppm NO/N₂ tanks. When the NO tank was set to deliver 50 ppm of NO, NO₂ concentration in the inspiratory limb of the circuit by CAPS monitoring was 0.13 at an FiO₂ of 0.21, 0.18 at an FiO₂ of 0.3, and 0.18 at an FiO₂ of 0.41. When NO delivery was increased to reach an inhaled concentration of 250 ppm of NO, NO₂ was 1.57 at an FiO₂ of 0.21, 2.35 at an FiO₂ of 0.3, and 2.61 at an FiO₂ of 0.41. By the electrochemical methodology, at 50 ppm of NO, the NO₂ concentration measured was 0.1 at an FiO₂ of 0.21, 0.3 at an FiO₂ of 0.3, and 0.3 at an FiO₂ of 0.41. At 250 ppm of NO, the NO₂ concentration measured was 2.37 at an FiO₂ of 0.21, 3.17 at an FiO₂ of 0.3, and 3.61 at an FiO₂ of 0.41. Similar NO₂ values were found when 857 ppm NO/N₂ tanks were used (see Table 5). These data provide a reference for adjusting desired NO delivery in the designed system.

TABLE 5
Nitric oxide and oxygen flow (in liters/minute) were set to
obtain a desired NO inspiratory concentration (50, 150, and 250
ppm) at a desired FiO₂. Medical air flow at 15 Liters/min
was set in every setting. Two different tanks were used with
different NO concentrations (800 and 857 ppm).

| | | | |
|---|---|---|---|
| NO | NO₂ | NO flow | O₂ flow |
| (ppm) | (ppm) | (L/min) | (L/min) |

NO/N₂ tank concentration = 800 ppm

| | | | | |
|---|---|---|---|---|
| FiO₂ = | 49 | 0.13 | 1 | 0.36 |
| 0.21 | 145.5 | 0.72 | 3.46 | 1.11 |
| | 249.5 | 1.57 | 6.99 | 2.10 |
| FiO₂ = | 48.5 | 0.18 | 1.2 | 3.50 |
| 0.30 | 140 | 0.97 | 4.25 | 4.82 |
| | 250 | 2.35 | 9.10 | 7.30 |
| FiO₂ = | 48 | 0.18 | 1.40 | 7.12 |
| 0.41 | 145 | 1.17 | 5.08 | 10.20 |
| | 241.5 | 2.61 | 11.91 | 17.4 |

NO/N₂ tank concentration = 857 ppm

| | | | | |
|---|---|---|---|---|
| FiO₂ = | 47.50 | 0.24 | 0.92 | 0.58 |
| 0.21 | 143.50 | 0.77 | 3.07 | 1.16 |
| | 237.50 | 1.98 | 6.18 | 2.37 |
| FiO₂ = | 49 | 0.26 | 1.05 | 3.46 |
| 0.30 | 143 | 1.24 | 3.84 | 5.40 |
| | 253 | 2.79 | 7.66 | 7.22 |
| FiO₂ = | 40 | 0.29 | 1.22 | 8.48 |
| 0.42 | 142 | 1.40 | 4.48 | 10.74 |
| | 236 | 3.11 | 10.05 | 15.70 |

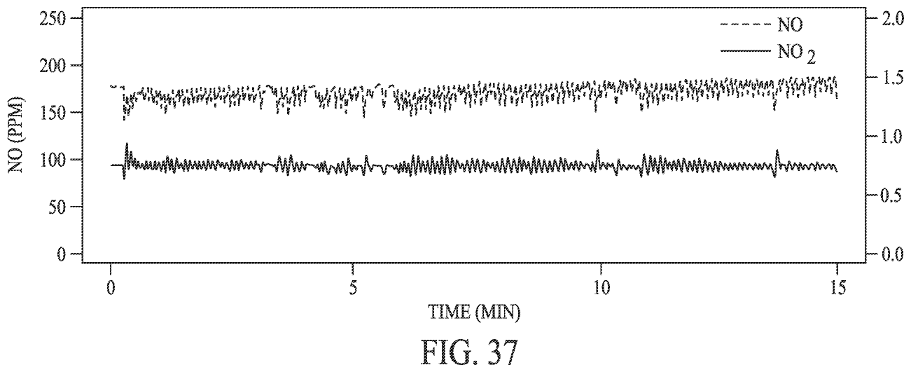

NO was administered to 5 healthy adult health care subjects: 2 males and 3 females. The median age was 32. The subjects had no history of cardiovascular or lung disease. The total number of NO administrations was 48. The average concentration of inspired NO was 164.8±10.74 ppm with NO₂ levels of 0.7=0.13 ppm; these levels remained stable throughout the administration (see FIG. 37). Oxygen was administered to keep the FiO₂ at 0.21 (see Table 5). During 15 min of administration of gaseous NO, methemoglobin levels increased from a baseline value of 1.05±0.58% to 2.26±0.47%. The subjects did not experience any discomfort during the procedure. No adverse events were reported. Despite the small number of administrations, these results indicate that breathing high concentration of NO for short periods of time using the newly developed NO breathing system is feasible and well tolerated without adverse events.

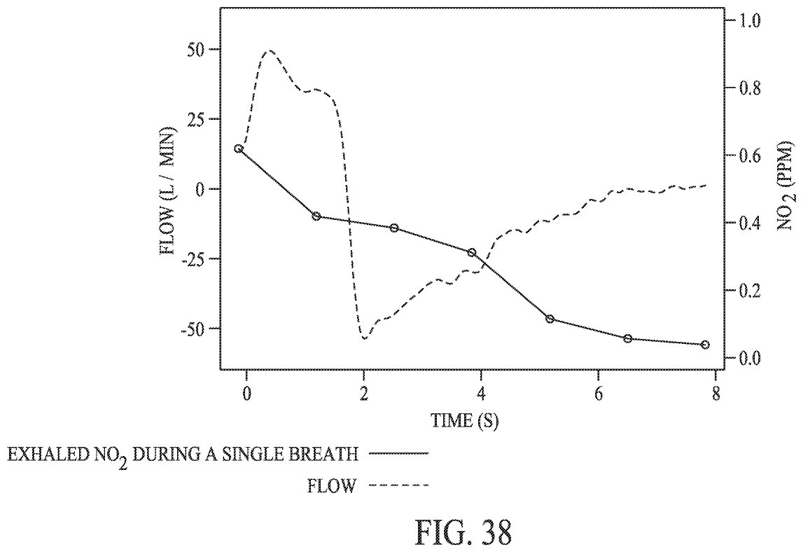

The average inspired NO and NO₂ concentrations were 153 ppm and 0.51 ppm, respectively, using an FiO₂ of 0.205. At the end of exhalation, NO₂ concentration decreased to 0.03 ppm (see FIG. 38). Exhaled FiO₂ was 0.195.

A device was built to reliably administer a high concentration of NO in spontaneously breathing subjects without the need for a mechanical ventilator. The use of a 3-Liter reservoir and high medical air flow helps this system to maintain a stable concentration of NO throughout a wide range of tidal volumes (from 0.25 to 1 L) independently form the source of gaseous nitric oxide. This system administers a concentration of NO up to 250 ppm at a range of FiO₂ from 0.21 to 0.4. Using this system, NO₂ levels are maintained at the prescribed low levels. Over the last few years, there has been increased interest in administering high-dose NO to spontaneously breathing patients outside the ICU setting and, possibly outside the hospital environment. Previous in vitro and in vivo evidence support the use of high-dose of NO as an antimicrobial. Over the past two years, a teenage patient was treated with 46-intermittent inhalations of 160 ppm NO for antibiotic resistant Burkholderia ultivorans lung infection in the setting of cystic fibrosis. No adverse events or delivery system failures were observed, and respiratory symptoms of the patient improved while the antibiotic pattern of the Burkholderia multivorans changed to allow common antibiotic coverage (e.g., Bactrim and Levofloxacin). Additionally, inhaled NO therapy has also shown to be a potent anti-inflammatory agent, reducing lung thrombosis after lung transplant.

In the setting of the current COVID-19 pandemic, administration of high-dose NO in spontaneously breathing COVID-19 patients is being tested to determine whether or not it leads to a reduced rate of hospital admission and respiratory failure requiring intubation and mechanical ventilation. Furthermore, a case series of 6 COVID-19 positive pregnant patients that received high dose (160-200 ppm) nitric oxide using the delivery system has also been tested.

To avoid overwhelming COVID-19 systemic inflammation, systems and methods to deliver NO treatment early were designed. Therapy with NO inhalation starts in the emergency department or upon hospital admission to the general care wards. One could envision treatments with NO gas with a similar prototype of NO delivery system for home use. The system that was designed and evaluated in this example allows for the administration of NO outside the ICU setting without a mechanical ventilator. If evidence continues to mount that high-dose inhaled NO is an effective anti-inflammatory, anti-thrombotic and antimicrobial agent, the system described can be used to treat or prophylactically treat many patients without the need for a dedicated mechanical ventilator. It has potential applications on the front lines, in an emergency room or rural clinic setting, or in low-resource settings, in the face of a pandemic due to a susceptible respiratory pathogen. The system is reproducible, inexpensive, reliable, and easy to build and maintain.

In conclusion, an NO delivery system that was built provides an alternative to a ventilator-based system to give high dose NO to spontaneously breathing patients. This system can efficiently administer high concentrations of NO via a comfortable fitting mask

### Example 5

Treatment options are limited for patients with respiratory failure due to coronavirus disease 2019. Conventional oxygen therapy and awake proning are options, but the use of high-flow nasal cannula and continuous positive airway pressure are controversial. There is an urgent need for effective rescue therapies. This example aims to evaluate the role of inhaled nitric oxide 160 ppm as a possible rescue therapy in non-intubated coronavirus disease 2019 patients. Retrospective evaluation of coronavirus disease 2019 patients in respiratory distress receiving nitric oxide gas as a rescue therapy. Coronavirus disease 2019 patients at high risk for acute hypoxemic respiratory failure with worsening symptoms despite use of supplemental oxygen and/or awake proning. Patients received nitric oxide at concentrations of 160 ppm for 30 minutes twice per day via a face mask until resolution of symptoms, discharge, intubation, or the transition to comfort measures only. Between Mar. 18, 2020, and May 20, 2020, five patients received nitric oxide inhalation as a rescue therapy for coronavirus disease 2019. All received at least one dosage. The three patients that received multiple treatments (ranging from five to nine) survived and were discharged home. Maximum methemoglobin concentration after 30 minutes of breathing nitric oxide was 2.0% (1.7-2.3%). Nitrogen dioxide was below 2 ppm. No changes in mean arterial pressure or heart rate were observed during or after nitric oxide treatment. Oxygenation and the respiratory rate remained stable during and after nitric oxide treatments. For two patients, inflammatory marker data was available and demonstrated a reduction or a cessation of escalation after nitric oxide treatment. Nitric oxide at 160 ppm may be an effective adjuvant rescue therapy for patients with coronavirus disease 2019.

Infection with severe acute respiratory syndrome coronavirus 2 (SARS-CoV-2) leads to coronavirus disease 2019 (COVID-19) and has various clinical manifestations, ranging from asymptomatic, or mild cold-like symptoms, to critical life-threatening conditions, including respiratory failure, septic shock, and/or multiple organ dysfunction. More than 75% of hospitalized COVID-19 patients require supplemental oxygen, and 17% to 35% of hospitalized patients are treated in an ICU, predominantly due to respiratory failure. Targeting an improvement in oxygenation, first-line treatment strategies include awake proning and supplemental oxygen. For additional oxygen delivery, nasal cannulas and nonrebreather masks are commonly used. The use of continuous positive airway pressure (“CPAP”) or high-flow nasal cannula (“HFNC”) and other noninvasive ventilation strategies is controversial in patients with acute hypoxic respiratory failure, due to concerns of the increased risk of exposing healthcare workers to infectious aerosols. If respiratory status worsens, initiation of invasive mechanical ventilation may be required to prevent life-threatening hypoxemia and additional organ damage. However, many COVID-19 patients are elderly with multiple comorbidities and maintain “do not resuscitate” and/or “do not intubate” (“DNI”) orders, further limiting treatment options. For patients with imminent respiratory failure, independent of patients' healthcare directive status, inhaled nitric oxide (NO) is offered as a rescue therapy when requested by the responsible clinicians at an institution.

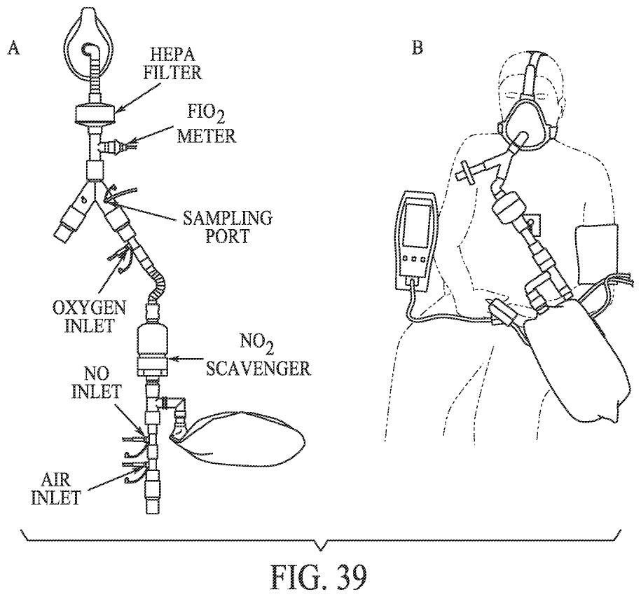

In this example, a case series is presented of spontaneous breathing severe COVID-19 adult patients that, due to clinically deteriorating respiratory conditions despite best practice, received 160 parts per million (ppm) NO for 30 minutes twice per day as a rescue therapy. Inhaled NO therapy was offered as an innovative rescue therapy for COVID-19 patients with rapidly progressive hypoxemic respiratory failure. These particular patients were not considered eligible for other rescue therapies within or outside clinical trials. Patients who received NO gas treatment as a rescue therapy, requested by managing clinicians, were retrospectively reviewed by the study team. Demographic and clinical data (cardiopulmonary function and laboratory results) were recorded. The oxygenation was expressed as peripheral oxygen saturation (SpO₂)/FiO₂ as a surrogate marker of PaO₂/FiO₂. NO was delivered at 160 ppm twice a day for 30 minutes with a custom-made delivery device for spontaneously breathing patients using a well-fitting mask (see FIG. 39). Currently, there are no guidelines for the use of NO for COVID-19 infections. The system to deliver NO gas was able to maintain the level of nitrogen dioxide, a toxic byproduct of NO gas, below the safety limit of 2 ppm. Methemoglobin, a type of hemoglobin derived by the reaction between NO and hemoglobin, was monitored noninvasively using CO-oximetry (Radical 7; Masimo, Irvine, Calif.) (see FIG. 39).

Between Mar. 18, 2020, and May 20, 2020, the response team was called upon to provide NO inhalation as rescue therapy for five patients at Massachusetts General Hospital. All patients had multiple comorbidities. None of these patients received remdesivir due to a lack of availability or a clinical contraindication. The patients' clinical characteristics are shown in Table 6.

TABLE 6
Baseline Characteristics and Treatment Course of patients with severe
coronavirus disease 2019 treated with 160 parts per million NO.

| General | Patient-1 | Patient-2 | Patient-3 | Patient-4 | Patient-5 | Patient-6 |
|---|---|---|---|---|---|---|
| Age, years | 27 | 24 | 30 | 27 | 25 | 33 |
| Gravida | G1P0 | G2P1 | G1P0 | G1P0 | G3P1 | G1P0 |
| (Parity) | | | | | | |
| Gestational age | 18 + 3 | 29 + 4 | 25 | 40 | 36 + 1 | 32 + 4 |
| at admission, | | (twins) | | | | |
| weeks + days | | | | | | |
| BMI, KG/m² | 35.1 | 34 | 25.1 | 33.3 | 30.3 | 32 |
| Underlying | Obesity | Obesity | None | Obesity | Obesity | Obesity |
| chronic disease | | | | Sickle- | | |
| | | | | cell | | |
| | | | | disease | | |
| Known | None | Gestational | None | None | None | None |
| pregnancy | | Diabetes | | | | |
| complications | | | | | | |

COVID-19 characteristics

| | | | | | | |
|---|---|---|---|---|---|---|
| Days from onset | 7 | 7 | 7 | 3 | 3 | 14 |
| of symptoms | | | | | | |
| Known positive | Yes | No | Yes | Yes | No | Yes |
| SARS-CoV-2 | | | | | | |
| test | | | | | | |
| at admission | | | | | | |
| Days of | 1 | 0 | 5 | 4 | 0 | 9 |
| SARS-CoV-2 | | | | | | |
| positivity | | | | | | |
| at admission | | | | | | |
| Disease severity | Critical | Critical | Critical | Severe | Severe | Critical |
| by first NO | | | | | | |
| administration | | | | | | |
| Respiratory | Yes | Yes | Yes | Yes | Yes | Yes |
| Rate > 30/min | | | | | | |
| SpO₂ < 93% | Yes | Yes | Yes | No | Yes | Yes |
| Oxygen | Nasal | Nasal | Nasal | Venturi | Venturi | Nasal |
| supplementation | Cannula | Cannula | Cannula | Mask | Mask | Cannula |
| (Delivery | | | | | | |
| methods) | | | | | | |
| Oxygen | 6 L/min | 4 L/min | 4 L/min | 31% | 24% | 3 L/min |
| supplementation | | | | | | |
| (L/min or %) | | | | | | |
| Lung | Yes | Yes | No | Yes | Yes | Yes |
| infiltrates > 50% | | | | | | |
| Severe | Yes | Yes | No | No | No | Yes |
| respiratory | | | | | | |
| distress | | | | | | |
| Shock | No | Yes | Yes | No | No | No |
| Multiple | No | No | No | No | No | No |
| organ | | | | | | |
| dysfunction | | | | | | |

Maternal Outcome

| | | | | | | |
|---|---|---|---|---|---|---|
| Delivered to | No | Yes | No | Yes | Yes | |
| date | | | | | | |
| Gestational | — | 30 | 36 + 2 | 40 | 36 + 1 | 38 + 2 |
| age at delivery | | | | | | |
| Type | — | C-Section | Vaginal | Vaginal | C- | C- |
| | | | | | Section | Section |
| Intubated | Yes | Yes | No | No | No | No |
| during hospital | | | | | | |
| stay | | | | | | |
| MV | 13 | 1 | — | — | — | — |
| Duration, days | | | | | | |
| NO sessions, n | 5 | 3 | 18 | 2 | 4 | 7 |
| NO dose, | 160 | 160 | 160 | 180 | 160 | 200 |
| ppm-Median | [160-160] | [160-200] | [160-160] | [160-180] | [160-160] | [200-200] |
| [Range] | | | | | | |
| Remdesivir, | 0 | 7 | 3 | 0 | 0 | 0 |
| days | | | | | | |
| Hospital LOS, | 25 | 9 | 12 | 2 | 6 | 6 |
| days | | | | | | |
| ICU LOS, days | 16 | 5 | 4 | 0 | 0 | 4 |
| Last available | Negative | Positive | Negative | Negative | Negative | Negative |
| SARS-CoV-2 | | | | | | |
| f/u test result | | | | | | |
| Days since | 23/16 | 28/28 | 17/9 | 26/22 | 21/21 | 23/14 |
| first positive | | | | | | |
| test? NO | | | | | | |
| initiation | | | | | | |

^{a}indicated that the patient received only one treatment and
^{b}indicated that the patient received only one treatment and methemoglobin remained below 3%.

Patients 1, 2, and 3 received between five to nine treatments in total. The median methemoglobin level at baseline was 0.3% (0.1-0.7%), and the peak value after 30 minutes of NO treatment was 2.0% (1.7-2.3%). Average peaks of methemoglobin of each patient are displayed in Table 6. Five minutes after cessation of the treatment, methemoglobin was 1.6% (1.0-2.0%). The three patients subjectively reported an improvement in breathing. The patients rested comfortably and, at times, fell asleep during treatment.

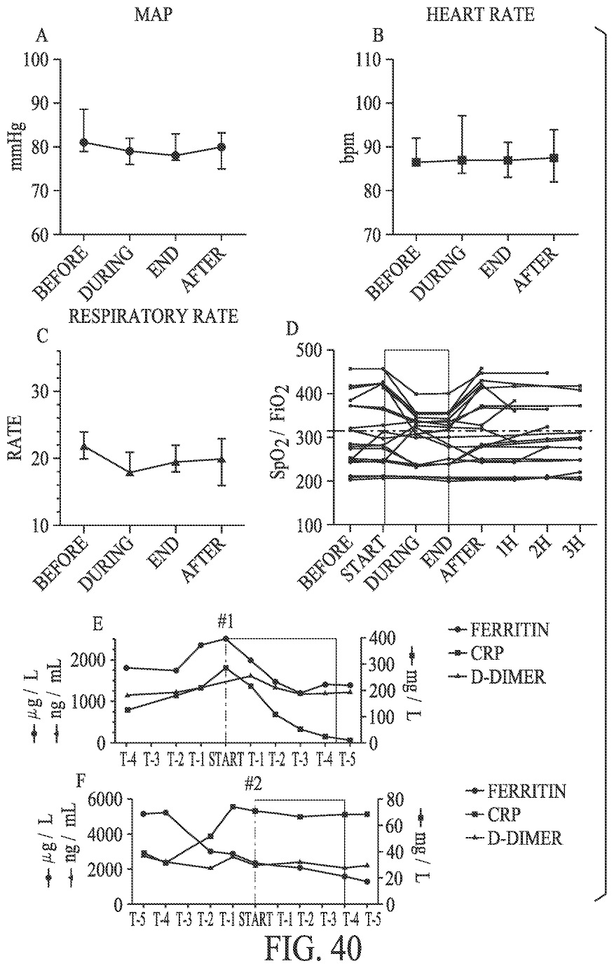

Two patients (patient 4 and patient 5) presented with severe respiratory distress, near respiratory failure and unable to maintain peripheral oxygen saturation above 90%, despite high supplemental oxygen (10 and 12 L/min) before NO gas started. Both patients were transitioned to comfort measurements within 24 hours after initiation of the first, and only, NO treatment. As shown in FIG. 40, no changes in mean arterial pressure or heart rate were observed during and after NO treatment. The respiratory rate did not change during or after the treatment (see FIG. 40). Oxygenation, expressed as SpO₂/FiO₂, remained stable during and after the treatments, whether the patient was initially hypoxemic or not (see FIG. 40). For patient 1, inflammatory markers decreased after the initiation of NO treatment (see FIG. 40). In patient 2, the inflammatory markers appear to stop rising after start of NO (see FIG. 40).

Inflammatory marker data from patient 3 are lacking. The two patients who were transitioned to comfort measurements only ultimately died. Patient 3 was intubated for 9 days and was later safely extubated and discharged from the hospital. The length of stay for Patients 1, 2, and 3 was 9, 10, and 28 days, respectively.

In this study, the administration of 160 ppm NO as rescue therapy was reported for non-intubated spontaneous breathing patients with severe COVID-19. Careful monitoring of vital signs did not demonstrate a systemic effect on blood pressure, heart rate, or respiratory rate, suggesting that a 30-minute treatment with 160 ppm does not adversely affect cardiopulmonary function in severe COVID-19 patients. Oxygenation during and after the treatment, and bedside monitoring of methemoglobin, showed that delivery of 160 ppm NO is well-tolerated, including treatment of patients with severe COVID-19. Patients tolerated the treatment exceptionally well and were often able to rest during inhalation, and their breathing effort was observed to decrease. A possible anti-inflammatory property of NO is reflected in patients 1 and 2, but further data are needed to draw a definitive conclusion.

All patients described here presented with comorbidities and had experienced worsening of symptoms over the days preceding initiation of NO therapy, with increased respiratory distress and increased supplemental oxygen demand despite the use of early proning. In noninfectious diseases, noninvasive ventilation, including CPAP or HFNC treatment, is a viable rescue treatment option, but at present, there is no evidence that using noninvasive ventilation leads to better outcomes in SARS-CoV-2 respiratory distress. In addition, noninvasive support is widely considered to generate infectious aerosols, leading to the spread of the virus, posing a high risk of exposure to healthcare workers. The respiratory guidelines at the institution restricts the use of HFNC and CPAP/bi-level positive airway pressure for the treatment of COVID-19 patients, further reducing supportive therapy options. In patients with multiple comorbidities, who are not eligible to be intubated and placed on mechanical ventilation (e.g., DNI), the lack of a viable rescue option will most likely result in death. For patients presenting with imminent respiratory failure, NO treatment might be an option, which can easily be combined with proning and antiviral treatment to slow the progression of COVID-19 infection and further clinical deterioration. Reducing hypoxia and enhancing bronchodilation may achieve some relief and alleviate suffering in a patient with COVID-19 who is otherwise comfort measures only.

NO inhalation at 10-80 ppm is Food and Drug Administration approved as a selective pulmonary vasodilator for hypoxic newborns with persistent pulmonary hypertension and is widely used off-label for adults with pulmonary hypertension and ARDS. NO-mediated bronchodilation in the setting of obstructive lung disease, as well as its anti-inflammatory effect, make NO a strong treatment option for respiratory distress due to COVID-19. Remdesivir treatment is not recommended in adults with impaired renal function and/or an elevation of liver enzymes. For patients not eligible for remdesivir treatment or for patients on remdesivir with respiratory symptoms, other therapeutic interventions may be necessary to prevent respiratory failure. In addition, a shortage of remdesivir is possible, and alternative treatment options are needed. It is reported that the administration of 160 ppm NO in non-intubated critically ill patients with COVID-19 is feasible and well-tolerated.

### Example 6

Rescue therapies to treat or prevent progression of coronavirus disease 2019 (COVID-19) hypoxic respiratory failure in pregnant patients are lacking. An aim was to treat pregnant patients meeting criteria for severe or critical COVID-19 with high-dose (160-200 ppm) nitric oxide by mask twice daily and report on their clinical response. Six pregnant patients were admitted with severe or critical COVID-19 at Massachusetts General Hospital from April to June 2020 and received inhalational nitric oxide therapy. All patients tested positive for severe acute respiratory syndrome coronavirus 2 (SARS-CoV-2) infection. A total of 39 treatments was administered. An improvement in cardiopulmonary function was observed after commencing nitric oxide gas, as evidenced by an increase in systemic oxygenation in each administration session among those with evidence of baseline hypoxemia and reduction of tachypnea in all patients in each session. Three patients delivered a total of four neonates during hospitalization. At the 28-day follow-up, all three patients were home and their newborns were in good condition. Three of the six patients remain pregnant after hospital discharge. Five patients had two negative test results on nasopharyngeal swab for SARS-CoV-2 within 28 days from admission. Nitric oxide at 160-200 ppm is easy to use, appears to be well tolerated, and might be of benefit in pregnant patients with COVID-19 with hypoxic respiratory failure.

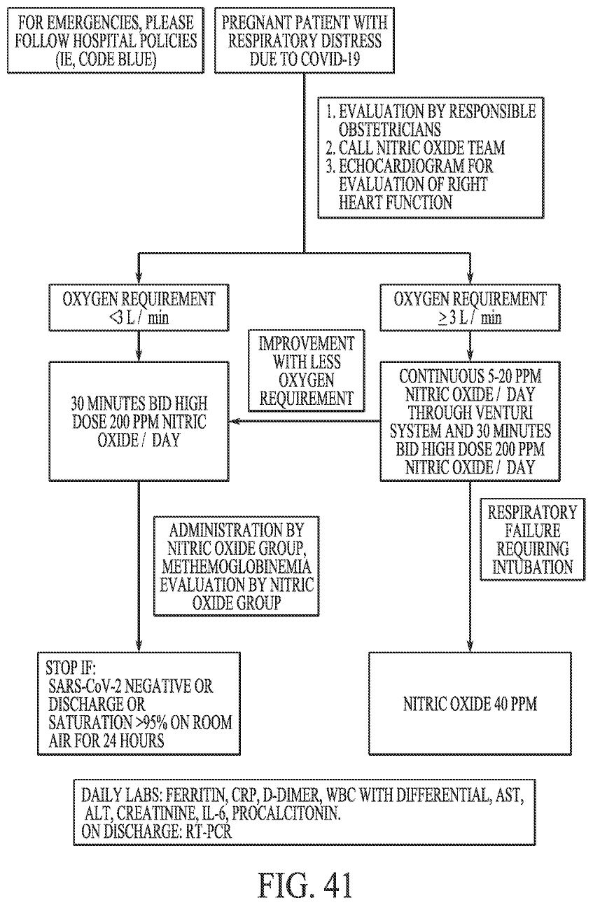

The treatment of pregnant patients with severe or critical coronavirus disease 2019 (COVID-19) with nitric oxide gas is reported in this example. Nitric oxide is a therapeutic gas that typically is delivered at 10-80 ppm to produce selective pulmonary vasodilation and improve arterial oxygenation. Owing to the efficacy of inhaled nitric oxide in adults with other causes of acute respiratory distress syndrome and pulmonary hypertension with a relatively safe therapeutic profile, an interdisciplinary team was formed to provide nitric oxide therapy to patients admitted to the hospital with severe or critical COVID-19 respiratory symptoms who were rapidly deteriorating (the nitric oxide treatment protocol is shown in FIG. 41). In the flowchart of FIG. 41, the following acronyms are used for the corresponding terms: COVID-19—coronavirus disease 2019; BID—twice a day; SARS-CoV-2—severe acute respiratory syndrome coronavirus 2; CRP—c-reactive protein; WBC—white blood cells; AST—aspartate aminotransferase; ALT—alanine aminotransferase; IL-6—interleukin-6; and RT-PCR—real time reverse transcription polymerase chain reaction. As shown in FIG. 41, the protocol includes determining an oxygen requirement, upon determining that the oxygen requirement is below a predetermined threshold (e.g., greater than or equal to 3 L/min), administering a concentration of nitric oxide gas greater than or equal to 150 ppm to a respiratory system of a pregnant patient for at least 30 minutes, and upon determining that the oxygen requirement is above the predetermined threshold, continuously administering a concentration of nitric oxide gas greater between about 5 ppm and about 20 ppm to the respiratory system of the pregnant patient and administering a concentration of nitric oxide gas greater than or equal to 150 ppm to the respiratory system of the pregnant patient for at least 30 minutes.

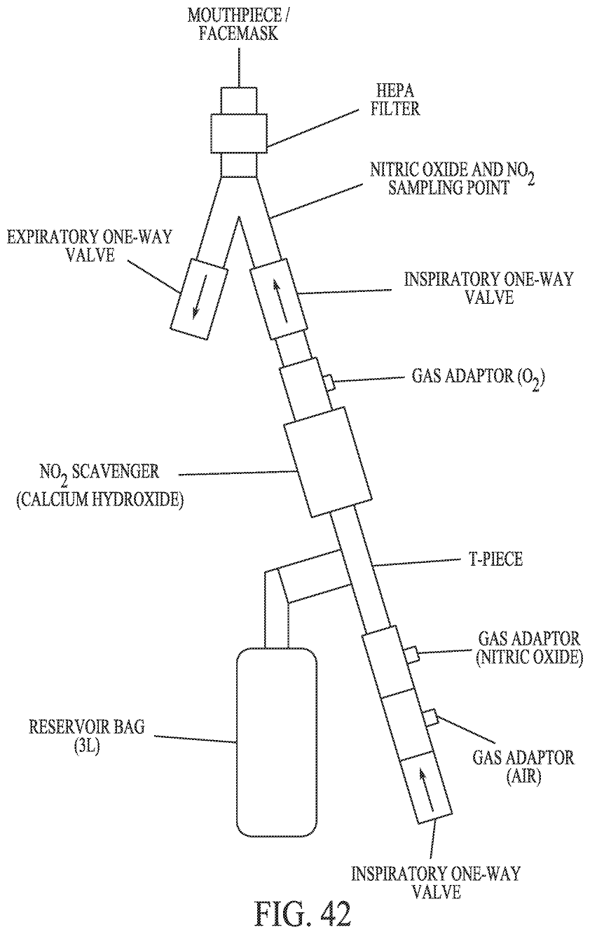

Patients were classified into two categories (severe or critical) according to the severity of respiratory, circulatory, and multiple organ involvement. An Institutional Review Board approved collection of the data from the Massachusetts General Hospital medical records. The nitric oxide gas was provided to spontaneously breathing, awake patients using a snug-fitting mask connected to the breathing circuit (see FIG. 42). The nitric oxide mixture was delivered at a concentration between 160 and 200 ppm at the desired FiO₂ concentration. Nitrogen dioxide concentrations had been measured previously using the same breathing circuit and a test lung in the laboratory and were maintained at concentrations below 1.5 ppm. Nitrogen dioxide is a toxic free radical gas derived from the reaction of nitric oxide with oxygen that can cause, at high concentrations, lung damage through direct epithelial cells injury. Treatment sessions lasted between 30 minutes and 1 hour. Before, during, and after each treatment session, SpO₂, methemoglobin saturation (%), heart rate, and noninvasive blood pressure were monitored continuously. During nitric oxide breathing, methemoglobin is formed by oxidation of iron (from Fe²⁺ to Fe²⁺). Ferric hemoglobin (MetHb [Fe³⁺]) is unable to transport oxygen. To avoid tissue hypoxia in respiratory failure, the goal was to maintain methemoglobin below 5%.

Data on the safety and efficacy of nitric oxide administration (SpO₂, heart rate, blood pressure, and methemoglobin) were reported in each patient's chart on completion of each session and are summarized as “baseline” (before starting nitric oxide delivery), “iNO” (approximately 15 minutes into the nitric oxide session), and “post-iNO” (5-10 minutes after nitric oxide delivery). Data are presented as the median and interquartile range or median (minimum and maximum values). All statistical analysis was conducted using Prism 8.4.2.

Six pregnant women with severe or critical COVID-19 were admitted to Massachusetts General Hospital from April 1 to Jun. 11, 2020. Nitric oxide inhalation was started in all patients as a rescue therapy on request by the treating clinical team within 48 hours after admission. Patient characteristics are shown in Table 7).

TABLE 7
Maternal Baseline Characteristics.

| General | Patient-1 | Patient-2 | Patient-3 | Patient-4 | Patient-5 | Patient-6 |
|---|---|---|---|---|---|---|
| Age, years | 27 | 24 | 30 | 27 | 25 | 33 |
| Gravida | G1P0 | G2P1 | G1P0 | G1P0 | G3P1 | G1P0 |
| (Parity) | | | | | | |
| Gestational age | 18 + 3 | 29 + 4 | 25 | 40 | 36 + 1 | 32 + 4 |
| at admission, | | (twins) | | | | |
| weeks + days | | | | | | |
| BMI, KG/m² | 35.1 | 34 | 25.1 | 33.3 | 30.3 | 32 |
| Underlying | Obesity | Obesity | None | Obesity | Obesity | Obesity |
| chronic disease | | | | Sickle- | | |
| | | | | cell | | |
| | | | | disease | | |
| Known | None | Gestational | None | None | None | None |
| pregnancy | | Diabetes | | | | |
| complications | | | | | | |

COVID-19 characteristics

| | | | | | | |
|---|---|---|---|---|---|---|
| Days from onset | 7 | 7 | 7 | 3 | 3 | 14 |
| of symptoms | | | | | | |
| Known positive | Yes | No | Yes | Yes | No | Yes |
| SARS-CoV-2 | | | | | | |
| test at admission | | | | | | |
| Days of | 1 | 0 | 5 | 4 | 0 | 9 |
| SARS-CoV-2 | | | | | | |
| positivity | | | | | | |
| at admission | | | | | | |
| Disease severity | Critical | Critical | Critical | Severe | Severe | Critical |
| by first NO | | | | | | |
| administration | | | | | | |
| Respiratory | Yes | Yes | Yes | Yes | Yes | Yes |
| Rate > 30/min | | | | | | |
| SpO₂ < 93% | Yes | Yes | Yes | No | Yes | Yes |
| Oxygen | Nasal | Nasal | Nasal | Venturi | Venturi | Nasal |
| supplementation | Cannula | Cannula | Cannula | Mask | Mask | Cannula |
| (Delivery | | | | | | |
| methods) | | | | | | |
| Oxygen | 6 L/min | 4 L/min | 4 L/min | 31% | 24% | 3 L/min |
| supplementation | | | | | | |
| (L/min or %) | | | | | | |
| Lung | Yes | Yes | No | Yes | Yes | Yes |
| infiltrates > 50% | | | | | | |
| Severe | Yes | Yes | No | No | No | Yes |
| respiratory | | | | | | |
| distress | | | | | | |
| Shock | No | Yes | Yes | No | No | No |
| Multiple organ | No | No | No | No | No | No |
| dysfunction | | | | | | |

Maternal Outcome

| | | | | | | |
|---|---|---|---|---|---|---|
| Delivered to | No | Yes | No | Yes | Yes | No |
| date | | | | | | |
| Gestational | — | 30 | 36 + 2 | 40 | 36 + 1 | 38 + 2 |
| age at delivery | | | | | | |
| Type | — | C-Section | Vaginal | Vaginal | Vaginal | Vaginal |
| Intubated | Yes | Yes | No | No | No | No |
| during hospital | | | | | | |
| stay | | | | | | |
| MV | 13 | 1 | — | — | — | — |
| Duration, days | | | | | | |
| NO sessions, n | 5 | 3 | 18 | 2 | 4 | 7 |
| NO dose, | 160 | 160 | 160 | 180 | 160 | 200 |
| ppm-Median | [160-160] | [160-200] | [160-160] | [160-180] | [160-160] | [200-200] |
| [Rangel | | | | | | |
| Remdesivir, | 0 | 7 | 3 | 0 | 0 | 0 |
| days | | | | | | |
| Hospital LOS, | 25 | 9 | 12 | 2 | 6 | 6 |
| days | | | | | | |
| ICU LOS, days | 16 | 5 | 4 | 0 | 0 | 4 |
| Last available | Negative | Positive | Negative | Negative | Negative | Negative |
| SARS-CoV-2 | | | | | | |
| f/u test result | | | | | | |
| Days since | 23/16 | 28/28 | 17/9 | 26/22 | 21/21 | 23/14 |
| first positive | | | | | | |
| test? NO | | | | | | |
| initiation | | | | | | |

MV = mechanical ventilation, LOS = length of stay, IQR = Interquartile range, and f/u = follow-up. Patients were classified as Severe if they presented with respiratory rate > 30 and lung infiltrates > 50%, while they were classified Critical if a severe organ insufficiency, identified as Severe respiratory distress, shock, or multiple organ dysfunction occur.

During the first 48 hours of hospitalization, all six patients required supplemental oxygen; four were treated in the intensive care unit for 3-17 days, and two were intubated for 30 hours-14 days. During mechanical ventilation, the intermittent high-concentration nitric oxide gas was suspended and continuous low-dose (less than 40 ppm) inhaled nitric oxide was delivered by the ventilator at the discretion of the intensive care unit team as per standard clinical protocols. A high concentration of nitric oxide gas treatments resumed after extubation. All patients were discharged home in stable condition (hospital length of 2-25 days) and were shown to be independent in their daily activities based on the Katz Index of Independence in Activities of Daily Living (see Table 8). at the 28-day follow-up.

TABLE 8
Activities of Daily Living, Mobility and
Instrumental Activities of Daily Living

| | Hospital | ICU | | Katz |
|---|---|---|---|---|
| Patient | length of | length of | Intubation | activities |
| # | stay [d] | stay [d] | time [h] | score at 28 days |
| 1 | 25 | 17 | 334 | 6 |
| 2 | 10 | 6 | 30 | 6 |
| 3 | 12 | 3 | 0 | 6 |
| 4 | 2 | 0 | 0 | 6 |
| 5 | 6 | 0 | 0 | 6 |
| 6 | 5 | 4 | 0 | 6 |

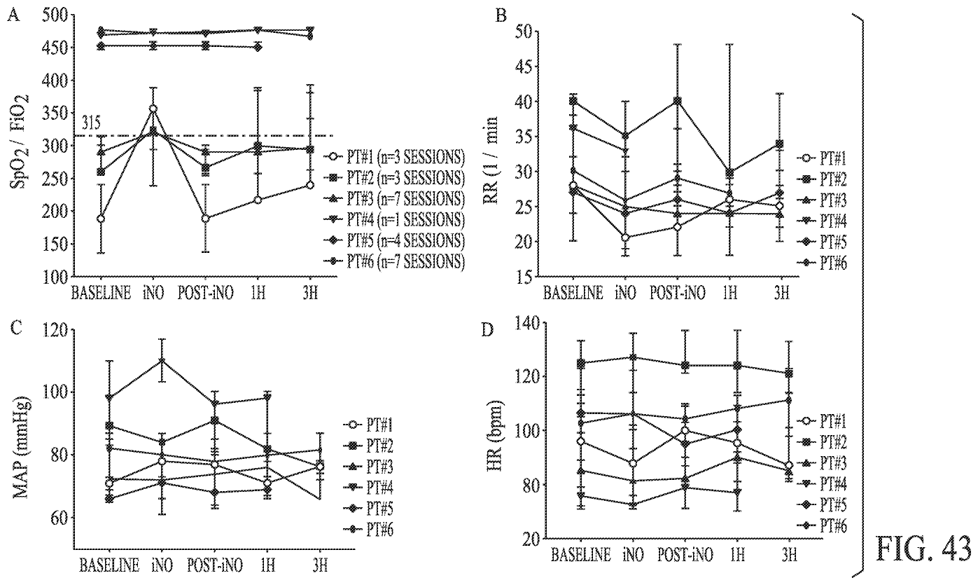

A total of 39 treatments of 160-200 ppm of nitric oxide gas inhalation were delivered to the six patients (ranging from 2 to 18 treatments per patient). Thirteen of the 26 treatments administered to patient 1, patient 2, and patient 3 occurred when baseline hypoxia was present (SpO₂%/FiO₂% ratio<315 is equal to [PaO₂]/FiO₂<300 mmHg). FIG. 43 illustrates the results of the treatments with values being presented as median [minimum-maximum] for each patient, with before iNO initiation, “iNO”: 15-minutes into the treatment, “Post-iNO”: 5-10 minutes after the end of the treatment, “1-h” and “3 h”: 1 and 3 hours after the end of NO. The shapes at iNO correspond to values during NO treatment. During the treatment, systemic oxygenation improved immediately during the delivery of nitric oxide in each of these treatment sessions (see FIG. 43 panel A). Nitric oxide inhalation was associated with rapid subjective relief of shortness of breath in all patients, and respiratory rates decreased (see FIG. 43 panel B), returning to being tachypneic 3 hours after the treatment (ranging from 0 to 16 hours after the treatment). Heart rate and mean systemic arterial pressure were unchanged compared with baseline (see FIG. 43 panels C and D).

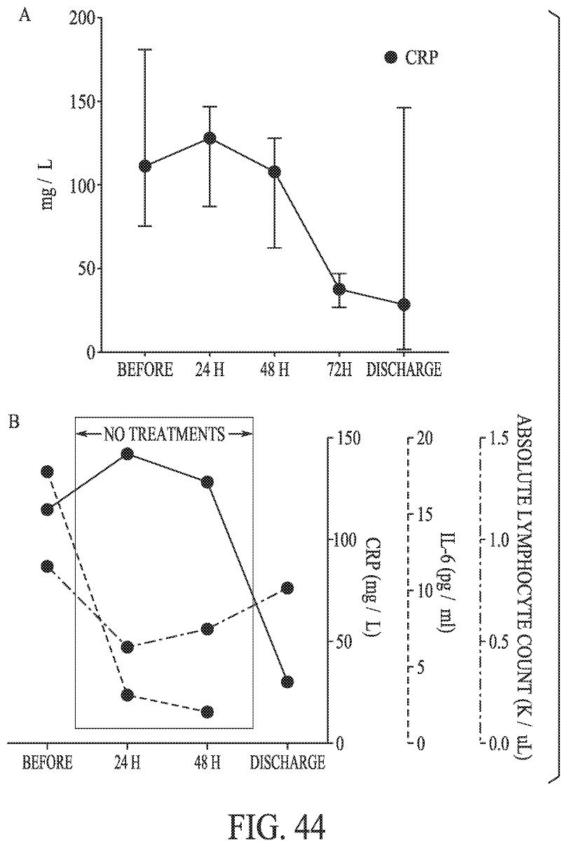

By the 28th day after initial hospitalization, five of six patients had tested negative twice for severe acute respiratory syndrome coronavirus 2 (SARS-CoV-2) by real time reverse transcription polymerase chain reaction from a nasopharyngeal swab, and one patient remained positive (see Table 7). C-reactive protein levels were measured daily in all patients, and levels decreased in association with the initiation of nitric oxide (see FIG. 44 panel A). In patient 6, initiation of nitric oxide therapy was associated with decline in C-reactive protein and interleukin-6 levels and a slow improvement in absolute lymphocyte counts (see FIG. 44 panel B). Inhalation of nitric oxide was well tolerated in all patients. No acute adverse effects were observed in any patient. Methemoglobin (baseline 0.9%, interquartile range 0.5-1.3%) was measured continuously and peaked at 2.5% (interquartile range 2.0-3.0%). During hospitalization, patients 4 and 5 received inhaled nitric oxide (5-20 ppm) and supplemental oxygen during delivery to maintain an SpO₂ level above 95%. Patient 4 delivered vaginally, and patient 5 delivered by cesarean.

Patient 2 required intubation for acute respiratory distress syndrome and hemodynamic shock and delivered twins by cesarean under general anesthesia at 30 weeks of gestation. She developed acute kidney injury (stage 2) the day after the emergent cesarean delivery (creatinine 1.12 mg/dL, with a baseline level of 0.51 mg/dL), and her kidney function returned to baseline value before discharge. Apgar scores were 2, 5, and 6 at 1, 5, and 10 minutes, respectively, for newborn A and 4 and 7 at 1 and 5 minutes, respectively, for newborn B. Birth weights were 1.34 and 1.60 kg, respectively. Both newborns were intubated and received surfactant at delivery and were extubated on day 3. All newborns tested negative on real time reverse transcription polymerase chain reaction for SARS-CoV-2 infection after delivery and remained in good condition by day 28 of maternal admission; the twins delivered at 30 weeks of gestation remain in the neonatal intensive care unit owing to prematurity, and the other newborns have been discharged home. Of the three women who remained pregnant at discharge, two have delivered without complications, although one had a late preterm birth at 36 weeks of gestation.

The treatment of severe or critical COVID-19 is difficult and not well studied. Although this is a preliminary report on the use of nitric oxide gas from a small cohort of spontaneously breathing pregnant patients with COVID-19, there are several plausible reasons for using nitric oxide in this population and newborns, including a reasonable safety profile; potential antiviral, anti-inflammatory, and mild bronchodilatory action; and selective pulmonary vasodilation, which may improve maternal and fetal oxygenation. Acute kidney injury is a known complication of nitric oxide treatment; one patient developed acute kidney injury, though a causal relationship between development of acute kidney injury and administration of nitric oxide cannot be derived from this data, and the patient had clinical events that may have contributed to the acute kidney injury, independent of nitric oxide treatments. Future patients treated with high-dose nitric oxide should be monitored for the development of acute kidney injury.

Inhaled nitric oxide gas has been used in pregnancy and has not been associated with teratogenicity. No adverse events were reported in the medical record or by the clinicians providing high dose nitric oxide to the patients (L.B., A.J.K., W.H.B., R.W.C., F.I., and M.G.C.). Transcutaneous methemoglobin levels peaked on average at 2.5%.

It is hypothesized that free-radical nitric oxide gas exerts virucidal action by direct nitrosation of critical virion proteins required for infection, replication, and transmission. However, the precise antiviral effects of nitric oxide have yet to be determined. In summary, high-dose inhaled nitric oxide was well tolerated and was associated with improved oxygenation and respiratory rate for pregnant patients with severe or critical COVID-19. The potential benefits of nitric oxide inhalation therapy to improve outcomes in patients with COVID-19 need to be tested in prospective randomized trials.

Up to 200 ppm of inhaled NO gas was administered to six pregnant patients with severe-critical COVID-19. NO gas has been shown previously not to be teratogenic. NO is quickly metabolized in the lungs into metabolites (e.g., mostly nitrate) and does not cause a reduction in systemic vascular resistance. In the lungs, NO gas increases right heart function by decreasing pulmonary vasoconstriction (1), improves oxygenation by improving ventilation/perfusion matching (2), kills the virus by nitrosation of the viral proteins, which prevents possible viral invasion in the placenta (3), induces bronchodilation improving ventilation (4), promotes anti-inflammation (5), and, ultimately, by raising maternal oxygenation, improves oxygen delivery to the physiologically low oxygen tension fetal circulation (6).

### Example 7

Inhaled nitric oxide (NO) is a selective pulmonary vasodilator and mild bronchodilator. This multicenter study evaluated the feasibility and effects of high-dose inhaled NO in non-intubated spontaneously breathing patients with Coronavirus disease-2019 (COVID-19). This is an interventional study to determine whether NO at 160 parts-per-million (ppm) inhaled for thirty minutes twice daily might be beneficial and safe in non-intubated COVID-19 patients. Twenty-nine COVID-19 patients received a total of 217 intermittent inhaled NO treatments for 30 minutes at 160 ppm between March and June 2020. Breathing NO acutely decreased the respiratory rate of tachypneic patients and improved oxygenation in hypoxemic patients. The maximum level of nitrogen dioxide delivered was 1.5 ppm. The maximum level of methemoglobin (MetHb) during the treatments was 4.7%. MetHb decreased in all patients 5 minutes after discontinuing NO administration. No adverse events during treatment, such as hypoxemia, hypotension, or acute kidney injury during hospitalization occurred. In the NO treated patients, one patient of 29 underwent intubation and mechanical ventilation, and none died. The median hospital length of stay was 6 days [interquartile range 4-8]. No discharged patients required hospital readmission nor developed COVID-19 related long-term sequelae within 28 days of follow-up. In spontaneous breathing patients with COVID-19, the administration of inhaled NO at 160 ppm for thirty minutes twice daily promptly improved the respiratory rate of tachypneic patients and systemic oxygenation of hypoxemic patients. No adverse events were observed. None of the subjects was readmitted or had long-term COVID-19 sequelae.

There are few respiratory therapeutic options for the acute symptomatic treatment of spontaneously breathing patients with pneumonia due to coronavirus disease 2019 (COVID-19). Supplemental inhaled oxygen, the use of high-flow nasal cannula (HFNC), awake prone positioning, continuous positive airway pressure, and non-invasive ventilation have been used in non-intubated COVID-19 patients. However, to date, none of these treatments has been shown to improve the clinical outcome of patients with COVID-19 pneumonia. Inhaled nitric oxide (NO) is a selective pulmonary vasodilator, approved by the Food and Drug Administration in 1999 at doses up to 80 parts per million (ppm) for the treatment of newborns with hypoxic respiratory failure due to persistent pulmonary hypertension. Inhaled NO is used as a rescue therapy to improve oxygenation of mechanically ventilated patients with severe acute respiratory distress syndrome (ARDS). Administration of NO gas induces bronchodilation, suppresses inflammation and thrombosis, and exerts antimicrobial effects.

A face mask apparatus allowing safe delivery of high-dose inhaled NO (up to 250 ppm) to spontaneously breathing patients was recently developed. In two recent case series, this apparatus administered 160-200 ppm NO gas to five COVID-19 patients with acute respiratory failure and to six pregnant women with severe and critical COVID-19 pneumonia. Inhaled NO improved oxygenation, reduced respiratory rate, and decreased plasma levels of inflammatory markers.

In this example, the safety and efficacy of breathing NO at 160 ppm, twice daily for 30 minutes in 29 spontaneously breathing, non-intubated, hospitalized patients with mild-to-moderate COVID-19-induced pneumonia was evaluated.

The patients received inhaled NO as part of a randomized clinical trial. Since the pre-specified randomized sample size (240) was not reached, we herein present the treatment group's data including the inhaled concentration of NO and nitrogen dioxide (NO₂), transcutaneous methemoglobin (MetHb) levels and vital signs before, during, and 5 minutes after ceasing NO treatment. Four indicators were used as safety endpoints: the incidence of MetHb elevation above 5% during NO treatment, the occurrence of rebound pulmonary hypertension (PH) following cessation of NO inhalation, the occurrence of acute hypotension or acute desaturation during the use of NO gas, and the proportion of patients who developed acute kidney injury (AKI). Serum creatinine was measured to detect AKI. AKI evaluation was included because previous studies suggested that renal injury may be associated with inhaling NO in patients with ARDS. The study also evaluated 6 outcome variables: 1) the incidence of tracheal intubation and mechanical ventilation within 28 days after study enrollment; 2) the 28-day all-cause mortality and number of days to clinical recovery, defined as the absence of fever, decreased respiratory rate to less than 24 breaths/min, absence of cough for three consecutive days, or hospital discharge; 3) hospital LOS, 4) hospital readmission rate, 5) a negative reverse transcriptase polymerase chain reaction (rt-PCR) for SARS-CoV-2 at 28 days after initiation of NO treatment, and 6) vital signs and selected plasma inflammatory markers. Vital signs and laboratory results were evaluated before the initiation of NO treatment and daily thereafter as the closest value to 8 am.

To visually assess pulmonary involvement, chest radiographs (CXRs) before NO treatment were evaluated by a subspecialty-trained thoracic radiologist with 23 years of experience. Adult patients admitted to the medical ward with 1) SARS-CoV-2 infection confirmed by rt-PCR assay, and 2) presence of COVID-19 symptoms such as cough or tachypnea (respiratory rate >24 breaths/minute) were included in the study. Patients on long-term oxygen therapy and those requiring HFNC therapy at the time of screening were excluded. Pregnant patients and patients with do not resuscitate, do not intubate, or comfort measures only orders were also excluded.

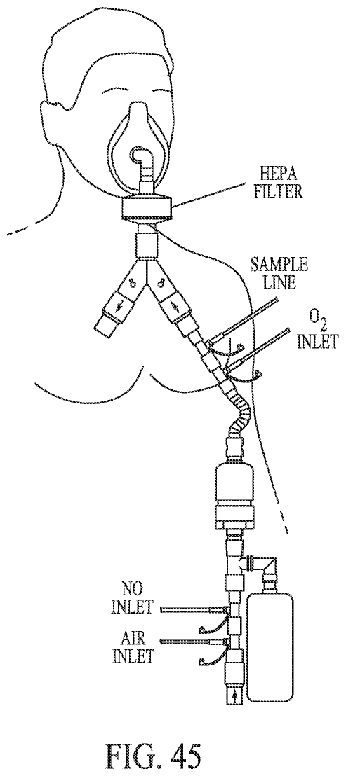

The patients received inhaled NO at 160 ppm for 30 minutes twice per day up to 14 days (28 treatments) or until hospital discharge, or a negative rt-PCR for SARS-CoV-2 (nasopharyngeal swab), or lack of respiratory symptoms for three consecutive days. Inhaled NO was delivered via the face mask apparatus previously reported (see FIG. 45), which had provided high-dose inhaled NO gas (up to 250 ppm) to spontaneously breathing patients. In FIG. 45, the NO delivery system included a non-rebreathing circuit that included standard respiratory circuit connectors, a reservoir bag, a scavenging chamber containing calcium hydroxide, and a snug-fitting mask was used to reach 160 ppm NO breathing. NO balanced in nitrogen (Praxair, Inc. Danbury, Conn., United States) was blended with medical air and oxygen to obtain the desired mixture, based on in vitro measurements. The delivery system allows stability of administered concentration of NO, with NO₂ levels always below the Occupational Safety and Health Administration (OSHA) threshold. Due to the COVID-19 hospital policy limiting the transfer of equipment between patients' rooms, it was not feasible to deploy more sensitive analytical methods (e.g. Sievers 280i Nitric Oxide Analyzer, GE Analytical Instruments, Boulder; CO and Cavity Attenuated Phase Shift [CAPS] NO₂ monitor (Aerodyne Research Inc, Billerica, Mass.)) to continuously measure the delivered NO and NO₂ concentrations during treatment. Before each administration, a bench test was performed to confirm the flows of air, oxygen, and NO gas necessary to obtain 160 ppm NO at a given fraction of inspired oxygen (FiO₂) and to assess the generated NO₂. The set flow of each gas was not changed during treatment.

To assess NO therapy's hemodynamic and respiratory effects, vital signs (respiratory rate, heart rate, and non-invasive blood pressure) were measured before treatment, 15 minutes after starting to breathe NO, and five minutes after each session. Peripheral oxygen saturation (SpO₂) was measured continuously from before treatment until five minutes after each session. The reaction between oxyhemoglobin and NO generates MetHb by oxidizing ferrous iron of the heme group to the ferric state. Since MetHb is unable to bind oxygen, a non-invasive transcutaneous MetHboximeter (Masimo rainbow SET, Irvine, Calif. 92618) was used to monitor MetHb levels during NO treatment continuously. It was considered that 5% MetHb as being the highest permitted level.

Rebound PH is a possible side effect upon discontinuation of inhaled NO. Patients were monitored for clinical and echocardiographic signs of rebound PH, including tachypnea, peripheral oxygen saturation (SpO₂)<80%, a decrease in mean arterial pressure (MAP) by >50 mmHg and an increased estimated right ventricular systolic pressure (RVSP) by >10%.

Data are presented as the median and interquartile range (IQR) for continuous variables and the number and percentage of instances for categorical variables. To evaluate the trend of a continuous variable over time (before, during, and after the treatment or during the hospitalization), a mixed effect model (R package [lme4] counting each patient as a random effect, R package [emmeans] for post hoc analysis) was used. To evaluate the trend of statistical significance was considered at a two-tailed P<0.05. All the analyses were conducted using R (R Core Team (2016). R: A language and environment for statistical computing. R Foundation for Statistical Computing, Vienna, Austria).

Between March and June 2020, 29 hospitalized patients were treated for COVID-19 with 160 ppm of inhaled NO gas twice daily. Study population characteristics are summarized in Table 9.

TABLE 9
Baseline Demographics and clinical characteristics.
Time to confirmed diagnosis = Time between onset
of symptoms and a confirmed diagnosis (positive SARS-CoV-2 test).
BMI = Body Mass Index; SpO₂ = Peripheral saturation
of oxygen; O₂ = Oxygen.

| | Baseline demographics and clinical | |
|---|---|---|
| | characteristics (n = 29) | |
| Age (years-old) | 50 | [41-60] |
| Gender, No. (%) Female | 13 | (44.8) |
| BMI (kg/m²) | 31 | [25-34] |
| Race, No. (%) | | |
| White | 27 | (93.1) |
| Black or African | 2 | (6.9) |
| American | | |
| Ethnicity, No. (%) | | |
| Hispanic or Latino | 22 | (75.9) |
| Not Hispanic or Latino | 7 | (24.1) |
| Comorbidities, No. (%) | | |
| Hypertension | 12 | (41.4) |
| Diabetes | 10 | (34.5) |
| Chronic Pulmonary | 3 | (10.3) |
| Disease | | |
| Time to confirmed diagnosis, | 6 | [3-10] |
| Days | | |
| Respiratory Rate, Breaths/min | 20 | [20-24] |
| SpO₂, % | 96 | [94-97] |
| O₂ requirement, No. (%) | 13 | (44.8) |
| O₂ requirement, L/min | 2 | [1.5-2.5] |
| Altered Chest Radiographs, | 24 | (82.8) |
| No. (%) | | |

The median time between the onset of COVID-19 symptoms and confirmed diagnosis by positive rt-PCR test was 6 [IQR 3-10] days. A total of 217 treatments were given to the 29 patients. The median number of treatments per patient was 6 [range 1-27] (see Table 10).

TABLE 10
Safety and feasibility. NO = nitric oxide. NO₂ =
nitrogen dioxide. FiO₂ = Fraction of inspired oxygen.
RVSP = Right Ventricular Systolic Pressure. Ppm =
parts per million. Min = minutes. Max = maximum value.

| | |
|---|---|
| Total number of | 217 |
| treatments, No. | |
| NO₂ inhaled, ppm | 1.1 [0.98-1.14], |
| | Max = 1.47 |
| FiO₂, % | 23 [21-30] |

Concerns

| Mask discomfort | | 3 | |
|---|---|---|---|
| Tingling of hands | | 1 | |
| bilaterally | | | |
| Nausea | | 1 | |
| | | | 5 minutes |
| | | | after |
| | Before | 15 minutes after | treatment |
| | Treatment | NO initiation | ends |
| Methemoglobin, % | 0.5 [0.2- | 1.8 [1.4- | 1.6 [1.2- |
| | 0.8] | 2.2] | 2.0] |
| | Max 2.3 | Max 4.7 | Max 3.5 |
| RVSP (mmHg) | 19.4 [15.4- | 17.9 [15.2- | 18.8 [16.6- |
| | 23.5] | 20.1] | 21.9] |

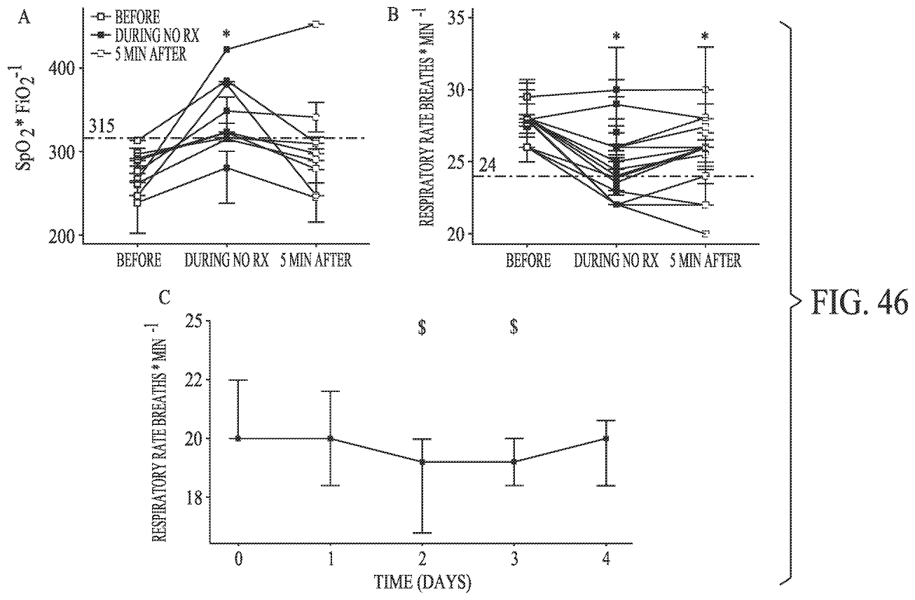

At the time of enrollment, 13 patients (45%) required supplemental oxygen (median 2 [IQR 1.5-3.5] L/min), and 24 patients (82%) had abnormal findings on CXR. Radiographic evaluation at the time of hospital admission showed in 16 patients (55%) bilateral, mid-to-lower-zone predominant and peripherally distributed lung opacities, which were considered classic for COVID-19. During the administration of NO, tachypneic patients had a decreased respiratory rate (see FIG. 46 panel A), demonstrating a role of NO gas in relieving respiratory distress. Several patients fell asleep and felt less distress. The ratio of SpO₂/FiO₂, a marker of systemic oxygenation, in the subgroup of patients who experienced pre-treatment hypoxemia (defined as an SpO₂/FiO₂<315), systemic oxygenation improved during NO gas administration (see FIG. 46 panel B). SpO₂(%)/FiO₂(%) improved by 49 [95% CI: 29-70] during treatment, and by 22 [95% CI: 1-42] after treatment in all patients with hypoxemia (SpO₂/FiO₂<315) before NO gas administration. The improved systemic oxygenation is likely due to improved ventilation to perfusion (V/Q) matching, due to selective pulmonary vasodilation by NO of ventilated lung regions or by bronchodilation. The RR was reduced by 2 [95% CI: 2-3] breaths/min during NO treatment (Rx) and remained reduced by 2 [95% CI: 1-3] breaths/min after treatment in all patients with tachypnea (RR at baseline >24 breaths/min) at the commencement of the administration.

During the first four days after randomization, the respiratory rate, starting from a median of 20 breaths/minute, decreased over time by 1 breath/minute each day of treatment. The largest difference was observed 48-72 hours after the initiation of NO therapy (see FIG. 46 panel C). The RR was reduced during hospitalization by 1 [95% CI: 0 to 1] breath/min for each day of treatment. At 48 h, the RR was lower by 3 [95% CI: 2-5] breaths/min. * P<0.05 vs Before. $ P<0.05 vs Day 0. The reduction in respiratory rate may indicate a resolution of respiratory symptoms possibly related to a lasting effect from multiple NO gas treatments. Mean arterial pressure and heart rate did not change during hospitalization. At the time of enrollment, patients had evidence of systemic inflammation with elevated plasma levels of Interleukin-6 (IL-6), C-reactive protein, ferritin, and D-dimers, while the median white blood cell counts were within the normal range. During hospitalization, reduced white blood cell count, C-reactive protein, and ferritin were not measured. An increase over time in platelet count within the normal range was observed. However, no definitive conclusions can be drawn related to the inhibition of intra pulmonary platelet aggregation by inhaled NO.

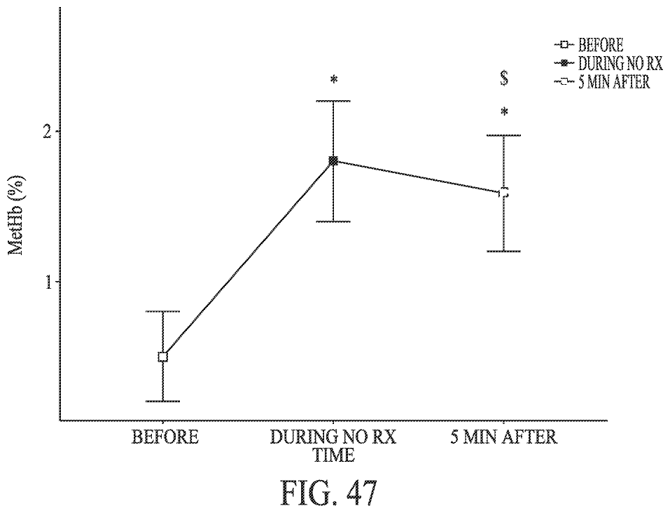

The maximum level of MetHb measured during NO treatment at 160 ppm was 4.7%; none of the treatments were terminated because of increased MetHb levels. MetHb levels rapidly decreased over 5 minutes in all patients after discontinuation of NO inhalation, suggesting normal red cell MetHb reductase activity in all patients (see Table 10 and FIG. 47). Transthoracic echocardiography (TTE) was performed in 14 patients receiving inhaled NO therapy. The estimated RVSP was normal in all patients before NO treatment (see Table 10). None of the patients developed clinical symptoms or echocardiographic signs of rebound PH after ending NO therapy. There were no hypotensive or hypoxemic episodes during NO inhalation. No device or system failures occurred during NO administration. However, three subjects reported discomfort due to the face mask, leading to discontinuation of their NO treatment. None of the patients developed AKI during hospitalization, suggesting that breathing 160 ppm NO gas for 30 minutes twice a day did not impair kidney function.

Only one patient out of 29 required intubation and mechanical ventilation due to progression of pulmonary disease. No deaths were observed among our population. Hospital LOS coincided with a clinical recovery time of a median of 6 days [IQR 4-8 days] (see Table 11). None of the patients subsequently required hospital readmission due to COVID-19 reactivation.

TABLE 11
Outcomes. MV = mechanical ventilation. Viral shedding identifies
viral clearance on rt-PCR from nasal or pharyngeal swab.

| | Outcomes | |
|---|---|---|
| Tracheal intubation and MV, | 1/29 | (3.4) |
| No. (%) | | |
| Mortality at 28 days, No. (%) | 0/29 | (0.0) |
| Time to clinical recovery, Days | 6 | [4-8] |
| Hospital length of stay, Days | 6 | [4-8] |
| Viral Shedding at 28 days, No. | 7/10 | (70) |
| (%) | | |
| Hospital readmission, No. (%) | 0/29 | (0.0) |

This study investigated the effectiveness and safety of inhaling 160 ppm NO gas twice daily for 30 minutes to reduce respiratory disease progression in hospitalized, non-intubated COVID-19 patients. Three significant findings emerge from this study. First, NO inhalation was associated with an acute reduction of respiratory rate in tachypneic spontaneously breathing COVID-19 patients. The reduction in respiratory rate was evident in patients with tachypnea at the beginning of NO treatment. Furthermore, the reduction of respiratory rate was sustained throughout hospitalization and until the time of discharge, suggesting a long-lasting beneficial effect of NO gas beyond selective pulmonary vasodilation, improved V/Q matching, and bronchodilation during treatment. Improved oxygenation was also observed in those patients who were hypoxemic before beginning each NO treatment. Second, the use of inhaled NO was associated with no hospital readmissions after discharge, this should be compared to a COVID-19-related readmission rate of 10-30% reported in the literature. Third, no adverse events occurred during NO inhalation. MetHb remained below the 5% safety limit in all patients. There were also no cases of AKI. These findings suggest that high-dose inhaled NO (160 ppm) can be safely administered to spontaneously breathing patients admitted with COVID-19.

The administration of NO to patients with COVID19-related pneumonia was associated with an acute reduction of respiratory rate and improved oxygenation. Bronchodilation due to NO may explain the acute decrease in the respiratory rate observed in our COVID-19 patients. In addition, patients receiving NO had a sustained decrease in respiratory rate throughout their hospital stay. Although no NO related systemic anti-inflammatory effects were measured, it is possible that intra-pulmonary anti-inflammatory and anti-thrombotic effects of repeated NO treatments may explain the beneficial effects of NO on lung function, in addition to the known improvement of oxygenation secondary to amelioration of V/Q matching.

The kinetics by which inhaled NO enters respiratory epithelial cells and produces antiviral effects are unknown. No readmissions were reported in the NO-treated patients. The observations in this example are encouraging when compared to the readmission rates reported in the United States and United Kingdom for patients admitted to hospitals with COVID-19, ranging from 10 to 30%. Of note, in a population of 33 patients admitted for COVID-19 at this institution, matched for age, gender, and severity, 6 subjects (19.4%) were observed to be readmitted within 28 days due to COVID-19 (data not shown). The decreased readmission rate in the NO-treated patients may have been due to the direct antiviral effect of inhaled NO.

Because of reduced assay availability early in the pandemic, only 10 patients underwent a follow-up test within 28 days from enrollment, and the results showed that 7 out of 10 treated and tested patients turned negative. Therefore, definitive results are unable to be provided regarding the effect of NO inhalation on SARS-CoV-2 clearance in patients with active COVID-19 pneumonia. In the present study, twice-daily administration of 160 ppm NO for 30 minutes in patients with mild COVID-19-associated pneumonia was found to be safe. This regimen produced a measurable beneficial effect on the respiratory system, without adverse events during the 217 treatment sessions. The echocardiographic and clinical evaluation did not show evidence of rebound PH. Pulse oximetric monitoring showed that MetHb levels did not rise above the 5% safety limit during or immediately following NO inhalation. In the patients with modest increases of MetHb, the level rapidly decreased within five minutes after completing NO therapy. These findings confirm previous case series regarding the safety of NO administration to healthy volunteers and pregnant COVID-19 patients. Nephrotoxicity has historically been a concern in patients with ARDS treated with NO. A meta analysis of data from four trials involving critically ill, mechanically-ventilated patients with septic ARDS (n=945) suggested an increased risk of AKI (relative risk 1.59, 95% CI 1.17 to 2.16). However, the explanation for the association between NO administration and the development of AKI is unknown. In contrast, a nephroprotective effect of NO treatment was observed in a separate meta-analysis, including 579 cardiac surgical patients. Notably, none of the patients receiving NO in this study developed AKI.

There are currently no pharmacologic interventions for COVID-19 that primarily target the respiratory system. NO gas therapy was observed to produce an acute improvement of systemic oxygenation in hypoxemic patients and reduced the respiratory rate. The rate reduction was both acute during NO therapy and sustained during the hospital stay. No re-hospitalization was observed among our population. The delivery of NO was found to be safe. These findings support the need for a randomized clinical trial to investigate the potential role of high-dose inhaled NO for the treatment of spontaneous breathing non-intubated COVID-19 patients.

### Example 8

Individuals with cystic fibrosis (CF) have persistent lung infections, necessitating the frequent use of antibiotics for pulmonary exacerbations. Some respiratory pathogens have intrinsic resistance to the currently available antibiotics, and any pathogen may acquire resistance over time, posing a challenge to CF care. Gaseous nitric oxide has been shown to have antimicrobial activity against a wide variety of microorganisms, including common CF pathogens, and offers a potential inhaled antimicrobial therapy. Here, the case of a 16-year-old female with CF is presented who experienced a precipitous decline in lung function over the prior year in conjunction with worsening antibiotic resistance of her primary pathogen, Burkholderia multivorans. She received 46 intermittent inhalations of 160 parts-per-million nitric oxide over a 28-day period. The gas was administered via a mechanical ventilator fitted with nitrogen dioxide scavenging chambers. High-dose inhaled nitric oxide was safe, well tolerated, and showed clinical benefit in an adolescent with cystic fibrosis and pulmonary colonization with Burkholderia multivorans.

Cystic fibrosis (CF) is a genetic disease characterized by recurrent lung infection and inflammation, which leads to progressive lung damage and respiratory insufficiency. Burkholderia multivorans (B. multivorans) belongs to a group of Gram-negative bacteria known as the Burkholderia cepacia (B. cepacia) complex. Bacteria from this group infect the lungs of only 2.4% of individuals with CF, but have inherent resistance to many common antibiotics, and colonization often leads to a decline in lung function. Nitric oxide (NO) plays a key role in host defense mechanisms in the lung and endogenous production of NO is decreased in CF airways. The US Food and Drug Administration (FDA) initially approved inhaled NO as treatment for pulmonary hypertension in newborns using 20 parts-per-million (ppm). Previous to this report, the use of high-dose inhaled NO in an individual with CF and pulmonary colonization with B. multivorans has not been described.

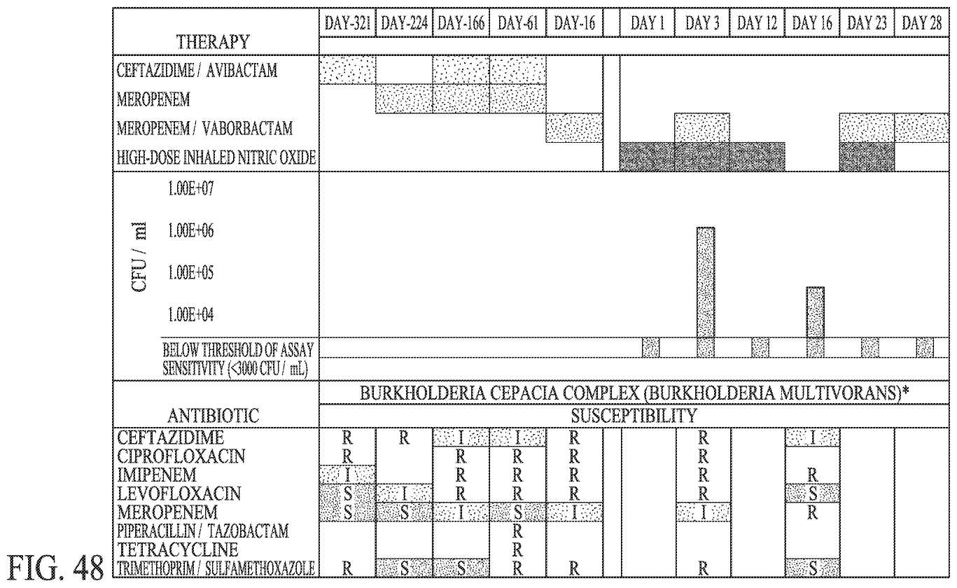

The patient is a 16-year-old female with CF (genotype: F508del/W1282X), CF-related diabetes requiring insulin, and CF-related liver disease after liver transplant, who experienced a precipitous decline in lung function over the prior year, requiring frequent-near monthly-hospital admissions for intravenous antibiotics targeting her primary respiratory pathogen, B. multivorans. The patient acquired this pathogen in her sputum six years before, and over time, it developed increasing antibiotic resistance (see FIG. 48). Notably, the patient required long-term immunosuppressive therapy following liver transplant 21 months before. Her frequency of hospital admission for pulmonary exacerbations remained unchanged for the first year after liver transplant but doubled in the succeeding 9 months. Her pulmonary exacerbations would only briefly respond to a single antibiotic, intravenous meropenem-vaborbactam. Furthermore, the patient's weight remained stagnant at the 10th percentile.

Female sex, body mass index <18 kg/m2, CF-related diabetes requiring insulin, B. cepacia complex infection, and frequent pulmonary exacerbations have all been identified as risk factors for mortality in individuals with CF and low lung function. Cystic fibrosis transmembrane conductance regulator (CFTR) modulators have been shown to increase lung function and decrease rates of pulmonary exacerbation in individuals with CF; however, the patient's genotype and history of liver transplantation precluded her from qualifying for any CFTR modulators available at the time or therapeutic trials, respectively. Lung transplantation was discussed though the patient's colonizing pathogen and adolescent age are controversially associated with poorer outcomes.

High-dose inhaled NO administration via a mechanical ventilator fitted with scavenging chambers was reviewed and approved under an institutional process called “Innovative Diagnostics and Therapeutics,” by which independent hospital leadership evaluate novel approaches to the care of individual patients. The patient assented, and her parents provided consent for this therapy.

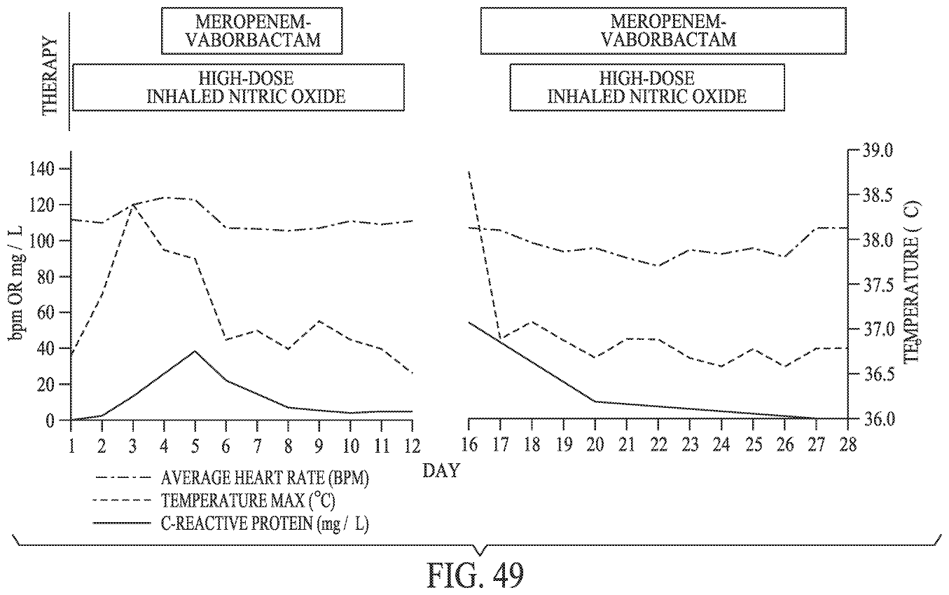

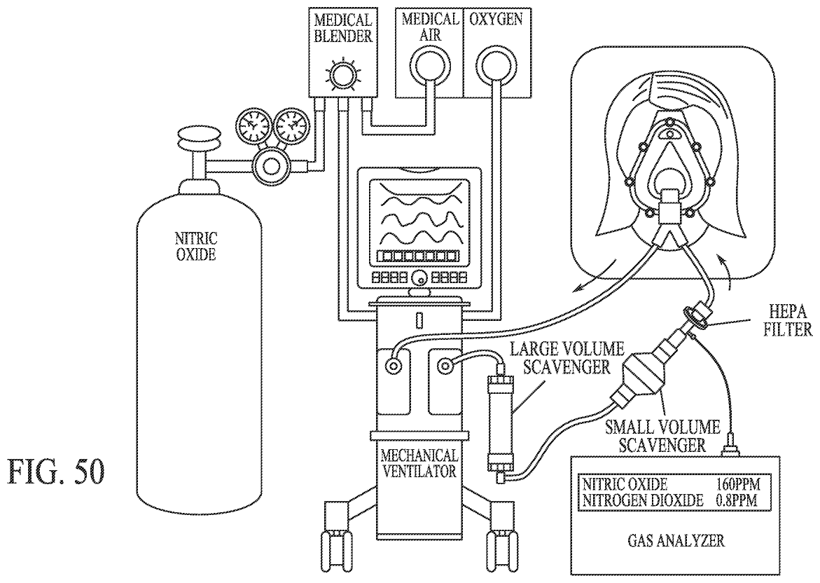

The patient received a total of 46 intermittent inhalations of 160 ppm NO over a 28-day period during two separate hospital admissions: days 1-12 and days 16-28 (see FIG. 49). During the first admission, inhaled NO was given over 30-minute intervals up to three times daily (27 total doses). In the second admission, the treatment interval was gradually increased to 60 minutes, two times daily (19 total doses), starting with 30-minute inhalations up to three times daily (9 doses), transitioned to 45-minute inhalations twice daily (2 doses), and ultimately 60-minute inhalations twice daily (8 doses). She concurrently received intravenous meropenem-vaborbactam (dose: 4 grams every 8 hours) on days 4-10 and 16-28. The NO gas source was commercially available as 850 ppm nitric oxide in nitrogen tanks that meet the Environmental Protection Agency (EPA) traceability protocol requirements (Airgas Inc., Radnor Township, Pennsylvania, USA). Oxygen, medical air, and NO were blended and introduced through a ventilator (Puritan Bennett 980 Series, Medtronic, Minneapolis, Minn., USA) (see FIG. 50). In FIG. 50, the nitric oxide gas source was an 850 ppm nitric oxide in nitrogen tank. This gas was mixed with medical air in a gas blender, and the resulting gas mixture was blended with oxygen in the mechanical ventilator. The blender and FiO₂ dial were both adjusted to achieve the targeted nitric oxide and oxygen concentrations. Large and small nitrogen dioxide scavengers were placed in series along the inspiratory limb. The gas mixture was delivered to the patient via a sealed facemask. Delivered gas was sampled just proximal to the patient, and nitric oxide and nitrogen dioxide concentrations were analyzed with a portable gas analyzer. A target level of 160 ppm inhaled NO and 0.21 FiO₂ were delivered via a tight-fitting noninvasive mask using a pressure support mode of 2cmH₂O driving pressure over a positive end-expiratory pressure of 5cmH₂O. Because the NO source of 850 ppm is mixed with nitrogen, the ventilator was set to a range of 0.25-0.26 FiO₂, in order to deliver an FiO₂ of 0.21 to the patient.

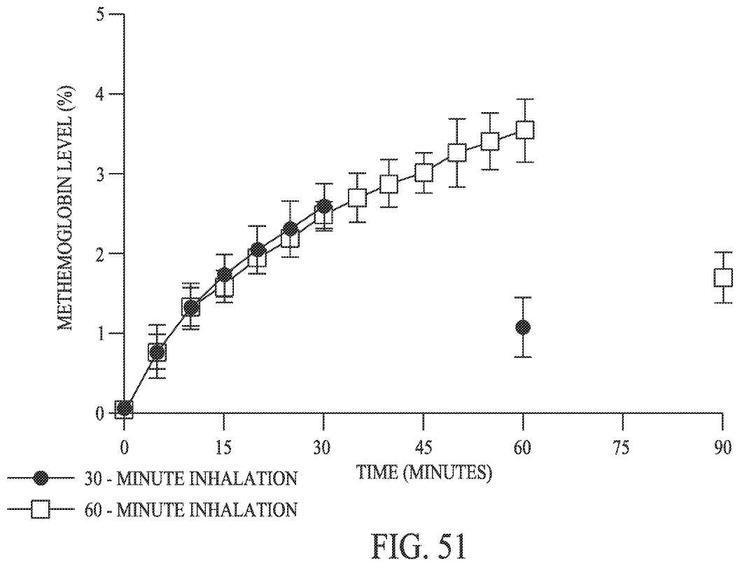

Nitric oxide and nitrogen dioxide (NO₂) analyzers were attached to a sampling port proximal to the facemask and provided real-time measurements. Nitrogen dioxide, a potentially harmful gas that develops when oxygen and NO are mixed, was scavenged with calcium hydroxide chambers (Spherasorb™, Intersurgical Ltd, Berkshire, UK), and levels remained less than 1.5 ppm in the circuit. Ambient NO and NO₂ levels were intermittently measured in the patient's room and consistently remained below EPA recommendations at less than 4 parts-per-billion (ppb) and 12 ppb, respectively. There were no serious adverse events, and inhalations were well tolerated. Echocardiograms were performed before, during, and after the first inhalation and demonstrated normal estimated pulmonary pressures. Heart rate, respiratory rate, oxygen saturation, and blood pressure were recorded at 5-minute intervals throughout inhalations and 30 minutes after cessation. The patient did not experience any hemodynamic instability or significant hypoxia with treatments. Nitric oxide oxidizes ferrous (Fe²⁺) hemoglobin into the ferric state (Fe³⁺), known as methemoglobin. Methemoglobin levels were continuously monitored during inhalations using a peripheral pulse co-oximeter (Masimo Rainbow Set™ Technology, Irvine, Calif.) and never surpassed the safety threshold of 5%. Specifically, at the end of the 30-minute and 60-minute inhalations, the highest recorded methemoglobin levels were 3.1% and 4.1%, respectively, and returned to baseline prior to subsequent treatments (see FIG. 51). In FIG. 51, the curves demonstrate a reproducible and expected rise in methemoglobin during therapy and reduction 30 minutes after cessation (note: data from 45-minute inhalations were omitted for clarity, but follow a similar rise, peak, and decrease 30 minutes post-inhalation). Additionally, the patient received 125 mg of ascorbic acid by mouth daily (on treatment days) to enhance methemoglobin reductase activity.

Sputum was collected prior to (days 1 and 16), midcycle (days 3 and 23), and on completion (days 12 and 28) of each NO cycle (see FIG. 48). Colony forming units (CFU) were calculated using sputum solubilized with Sputolysin® (MilleporeSigma) and plated on Burkholderia cepacia isolation agar. On days 1, 12, 23, and 28, B. multivorans grew from the patient's sputum, but the CFU count was low (see FIG. 48). Early in therapy (day 3), the sputum culture yielded highly antibiotic-resistant B. multivorans, consistent with months before; however, by the end of the first week of therapy (day 16), isolated B. multivorans demonstrated susceptibility to trimethoprim-sulfamethoxazole and levofloxacin and intermediate susceptibility to ceftazidime, a susceptibility pattern not seen for months.

Fever, white blood cell count, and C-reactive protein all decreased with therapy (see FIG. 49). The patient never manifested bacteremia or hemodynamic instability suggestive of cepacia syndrome—a dreaded complication of B. cepacia complex infection primarily linked to Burkholderia cenocepacia though also associated with B. multivorans. The patient's international normalized ratio (INR) remained consistently below 1.2, and she never demonstrated evidence of platelet dysfunction, hemoptysis, or other bleeding. Her lung function improved from her baseline. Specifically, on day 1—before starting high-dose inhaled NO therapy and after recently completing a 16-day course of intravenous meropenem-vaborbactam two days before—the patient's forced expiratory volume in 1 second (FEV1) and forced vital capacity (FVC) were 1.60 L (49% predicted) and 2.05 L (56% predicted), respectively. Both measurements improved at the end of the second admission to FEV1 1.72 L (52% predicted) and FVC 2.36 L (64% predicted). The patient's baseline weight in the prior 6 months remained at the 10th percentile, and her BMI was consistently below 18 kg/m². Her weight at the start of inhaled NO therapy was 48.4 kg (22nd percentile). It rose to a maximum of 50.3 kg (30th percentile) on day 16 but decreased to 48.9 kg (24th percentile) by day 24.

Here, the inpatient use and tolerability of high dose inhaled NO is described as an adjunct anti-infective targeting highly resistant B. multivorans in an adolescent with CF. There were no significant adverse events. An increase in the patient's pulmonary function and weight was observed relative to baseline.

Additionally, the antibiotic resistance pattern of B. multivorans improved, demonstrating susceptibility to trimethoprim-sulfamethoxazole and levofloxacin, where resistance to these antibiotics had previously been persistent. The change in the antibiotic resistance pattern after inhaled NO therapy is promising though challenging to interpret. Studies on the bactericidal mechanism of high-dose NO in Burkholderia bacteria are limited though given its proposed indiscriminate mechanism of membrane protein modification, DNA damage, and nitrosative and oxidative stress, and it is possible that high-dose NO exerted direct toxic effects on the bacteria contributing to a reduction in CFU and killing of more resistant isolates. An alternative hypothesis may be biofilm dispersal. Nitric oxide is thought to mediate dispersal by diffusing into biofilms and upregulating bacterial phosphodiesterases which inhibit or degrade the second messenger and biofilm regulator, cyclic-di-guanosine monophosphate (c-di-GMP). In this clinical case, it is possible that biofilms sheltered more antibiotic sensitive isolates of B. multivorans that were released into the planktonic state, and thus more susceptible to antibiotic-mediated killing after the initiation of high-dose inhaled NO.

Inhaled high-dose NO therapy, utilized as an anti-infective adjunct therapy, improved pathogen antibiotic resistance patterns and clinical outcomes in this patient. Mechanistic studies in vitro and larger randomized controlled clinical trials are needed to better understand the bactericidal efficacy and biofilm dispersal properties of high-dose NO on B. multivorans. This is believed to be the first report of high-dose inhaled NO therapy in an individual with CF and pulmonary colonization with B. multivorans. In follow-up, the patient continues to require frequent courses of antibiotics for pulmonary exacerbations though much less frequent inpatient admissions. During inpatient stays, high-dose inhaled NO therapy is continued to be offered as an adjunct therapy to intravenous antibiotics. The patient's genotype qualifies for the newly available, highly effective, triple combination CFTR modulator, elexacaftor-ivacaftortezacaftor (Vertex Pharmaceuticals), which has improved her lung function but has not yet altered her sputum microbiota. Recalcitrant infection with multidrug-resistant pathogens will likely continue to be a major problem in CF despite the advent of CFTR modulators, and high dose inhaled NO may help those who continue to struggle with difficult to treat respiratory pathogens.

### Example 9

In human hosts, SARS-CoV-2 causes a respiratory syndrome (named COVID-19) which can range from a mild involvement of the upper airways to a severe pneumonia with acute respiratory syndrome that requires mechanical ventilation in an intensive care unit (ICU). Hospital-associated transmission is an important route of spreading for the SARS-CoV-2 virus and healthcare providers are at the highest risk. As of February 2020, 1716, Chinese healthcare workers had confirmed SARS-CoV-2 infections and at least 6 died. A plan will be to randomize 470 healthcare providers scheduled to work with COVID 19 patients to receive nitric oxide gas administration (NO group, n=235) or no gas administration (control group, n=235). The primary endpoint of this study is the incidence of subjects with COVID-19 disease at 14 days from enrollment. Secondary endpoints are the proportion of healthcare providers who present a positive real time RT-PCR test for SARS-CoV-2 14 days after enrollment, the proportion of healthcare providers requiring quarantine, and the total number of quarantine days in the two groups.

The current COVID-19 pandemic started in December 2019 from Wuhan, China and rapidly diffused throughout the Asian continent, to Europe, and more recently to the United States. COVID-19 is predominantly a respiratory infection that spans from a mild involvement of the upper respiratory tract to a severe pneumonia leading to respiratory distress, shock, and death. The disease is commonly transmitted in hospital settings and healthcare providers have the highest risk of being infected. In February 2020, SARS-CoV-2 was confirmed in about 1,700 Chinese healthcare workers, and, on Apr. 4 2020, up to 189 Massachusetts General Hospital employees were infected by SARS-CoV-2. A safe and prophylactic therapy to reduce the instance of COVID-19 disease in healthcare workers would be of great benefit to clinical staff and society.

It is hypothesized that a high dose of exogenous inhaled NO is a virucidal agent in COVID-19 disease. Thus, it is hypothesized that the administration of high doses of nitric oxide will prevent the development of COVID-19 disease in healthcare providers exposed to SARS-CoV-2 positive patients.

This is a single center, randomized (1:1) controlled, parallel-arm clinical trial. It is proposed that this protocol be available to all interested centers with SARS-CoV-2 patients. In-hospital healthcare providers will be recruited. Candidates should be scheduled to work with confirmed SARS-CoV-2 patients for at least 3 days of the week. Exclusion criteria are proven previous SARS-CoV-2 infection and subsequent negative rt-PCR test, pregnancy, known hemoglobinopathies and known anemia. Inclusion and exclusion criteria are summarized in Table 12.

TABLE 12
inclusion and exclusion criteria. RT-PCR =
real time polymerase chain reaction.

Inclusion criteria
Age ≥18 years
Scheduled to work with SARS-CoV-2 RT-PCR
patients for at least 3 days in a week
Exclusion criteria
Proven previous SARS-CoV2 infection and
subsequent negative RT-PCR test
Pregnancy
Hemoglobinopathy
Anemia

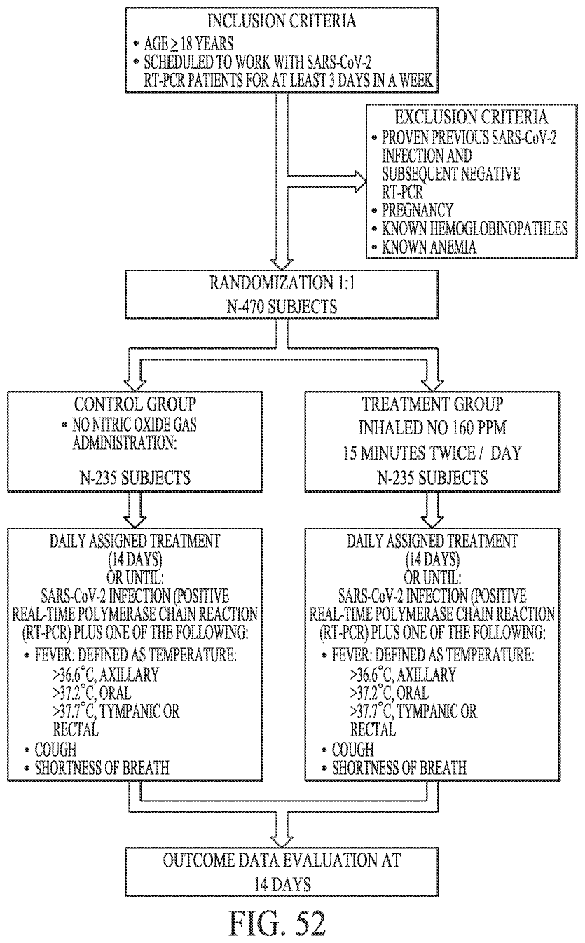

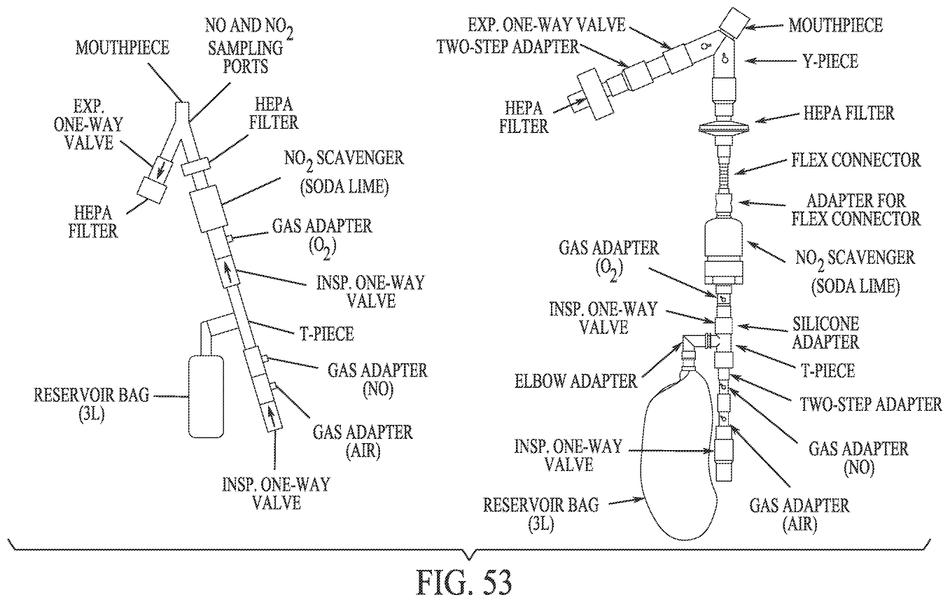

Eligible subjects will be randomized to receive nitric oxide administration (treatment group) or no gas administration (control group). NO will be delivered in 2 daily sessions (before and after the work shift) for 14 consecutive days. Each session will last 15 minutes, for a total of 30 minutes/day for each subject. See FIG. 52. NO gas will be administered through a mouthpiece connected to a breathing circuit (see FIG. 53). This circuit is composed by an inspiratory (with NO, air and oxygen inlet) and an expiratory limb. On the inspiratory limb there are (I) a 3 L reservoir bag and (II) a scavenger containing Soda Lime to minimize the NO₂ concentration administered to the subject. To avoid oxygen and NO mixing in the reservoir bag (leading to NO₂ formation) a one-way valve is positioned between nitric oxide and oxygen inlet. Inspiratory concentration of NO and NO₂ will be continuously monitored; NO₂ concentrations will be maintained below 2 ppm. Methemoglobin will also be continuously monitored non-invasively with a dedicated pulse oximeter system and will be maintained below 5%. NO delivery will be titrated in order to keep methemoglobin under 5%.

During the session, SpO₂ and heart rate will be continuously monitored by the study staff. Blood pressure will be measured non-invasively before and after the treatment. Values will be recorded in a dedicated paper data sheet, specifically: (I) before starting NO delivery, (II) at the end of the delivery, and (III) at 5 to 10 minutes after completion of the NO delivery to demonstrate a decrease of methemoglobin after cessation of gas delivery. Subjects assigned to the control group will not receive any gas therapy. Subjects will be monitored for 14 days in both groups. Breathing NO at 160 ppm for 30 minutes is safe. The theoretical risks of NO breathing include the following: pulmonary edema, methemoglobinemia, hypoxia, and hypotension.

In a corresponding recent study, there was no side effect or drug toxicity associated with the use of NO at 80 ppm for up to 24 hours. During nitric oxide gas administration, the study staff will continuously monitor the NO, NO₂, and oxygen concentrations in the inhaled gas. Peripheral oxygen saturation (SpO₂) will be continuously monitored. Methemoglobin levels will be assessed using a non-invasive continuous co-oximetry monitor. If the methemoglobin level rises above 5%, NO will be progressively reduced to reach a value <5%. Any study subject who experiences a side effect that is suspected to be related to the study drug will not be allowed to continue in the protocol.

No masking is going to be used in this study. Subjects and investigators will be aware of the study allocation. The primary endpoint of this study is the incidence of subjects with COVID-19 disease 14 days post-enrollment. COVID-19 is defined as a SARS-CoV-2 infection confirmed by a positive real-time polymerase chain reaction (RT-PCR) with fever (>36.6 C from the axillary site; or >37.2 C from the oral site; or >37.8 C from the rectal/tympanic site), cough, or shortness of breath. The secondary endpoints are the proportion of healthcare providers who present a positive real time RT-PCR test for SARS-CoV-2 14 days after enrollment, proportion of healthcare providers requiring quarantine, and total number of quarantine days in the two groups. Based on available data from Italy and China, it is predicted that a 15% incidence of SARS-CoV-2 infection among healthcare providers and it is assumed that the incidence will be reduced to 5% with inhalant NO cycles. Considering an alpha level of 0.05 and a power of 0.9, we determined a sample size of n1=207, N2=207, by a two-sided test. In order to account for possible dropouts, the sample size was increased by about 10% from 414 to a total of 470 subjects (235 each group). Estimated sample sizes were calculated using the Stata 14.1 software.

This study is targeted for a population of healthcare providers working in a hospital with COVID-19 patients. Upon admission of the first SARS-CoV-2 positive patient in a hospital unit, all the healthcare professionals that meet the inclusion and not the exclusion criteria become eligible for the study. The clinical staff of each unit will be adequately informed about the protocol details. No clinical care procedure will be interrupted or delayed due to the study procedure. Randomization will occur through a random allocation sequence generated by a computerized random generation program (REDCap). Parallel allocation to the treatment or control groups will occur with a 1:1 ratio. Randomization will not be stratified for pre-specified demographic conditions (e.g., sex, age). Results will be adjusted for such conditions in the analysis phase as specified in the statistical analysis section. Subjects will be randomly allocated to either the treatment group with NO administration or to the control group with no NO administration. Clinical information including medical history will be obtained from the participant interview. Collection of study variables will be managed by the outcome assessors using a dedicated subject's file on REDCap.

Outcome assessors, treatment providers, and the principal investigator will obtain unique usernames and passwords to transfer all data to a REDCap database dedicated to the study. Data will be analyzed following the intention to treat analysis principle. Demographic and clinical data will be presented as proportions for categorical outcomes and mean plus standard deviation or median plus interquartile range for continuous outcomes. Comparison between groups for the primary outcome will be made with the X2 test or the Fisher exact test for categorical variables. Secondary outcomes will be analyzed with the X2 test or the Fisher exact test for categorical variables and with the t-test or Wilcoxon rank-sum test for continuous variables after assessing the total number of quarantine days for normal distribution. A subgroup analysis will be performed using multiple linear regression, as well as logistic and cox regression models to adjust for age, pulmonary comorbidities, history of neoplastic disease, and the hour of exposition to SARS-CoV-2 positive patients in 14 days.

Data will be monitored by the principal investigator (PI) in collaboration with an independent Data and Safety Monitoring Board (DSMB). The PI and DSMB will monitor adverse events, evaluate data quality, and provide recommendations accordingly. The PI will monitor compliance to safety rules every 20 patients. Every violation will be reported to the DSMB. Before the beginning of the study, the DSMB will meet to decide safety rules and stopping Guidelines. The Data Safety Management Board (DSMB) will perform an interim analysis for superiority after the 25th patient. In case of a significant decreased infection rate in the treated subjects, the trial will be stopped. The signed informed consent will be kept in a secure place for at least 5 years after study completion.

Safety data includes levels of Methemoglobin, NO₂ levels, minor deviation from the protocol, and minor adverse reactions to NO gas administration. Other data to be reviewed includes the maintenance of patient confidentiality throughout the study. In accordance with PHRC policy on Adverse Event Reporting and Unanticipated Problems Involving Risks to Subjects, the Principal Investigator will report adverse events or other unanticipated problems to the DSMB and to the PHRC within 5 working days/7 calendar days of the date the investigator first becomes aware of the problem. Mild or moderate adverse events will be presented in progress reports at continuing reviews. The decision regarding altering or stopping the protocol will be performed by the principal investigator together with the Data Safety Management Board (DSMB). Protocol exit criteria will be: a. Acute worsening hypotension defined by a decrease in mean blood pressure of >20 mmHg not attributable to other causes such as the progression of the disease, hypovolemia, hemorrhage, sepsis, or acute heart failure. b. Sudden hypoxemia defined as a 5% reduction of peripheral oxygen saturation (SpO₂) from the basal value. c. Any life-threatening symptom potentially attributed to NO administration by the physician investigator.

### Example 10

High dose (greater than or equal to 80 parts-per-million [ppm]) inhaled nitric oxide (NO) has antimicrobial effects. A trial was designed to test the preventive effects of high dose NO on coronavirus disease (COVID-19) in healthcare providers working with COVID-19 patients. The study was interrupted prematurely due to the introduction of COVID-19 vaccines for healthcare professionals. Thus, present data on safety and feasibility of breathing 160 ppm NO using two different NO sources, namely pressurized nitrogen/NO cylinders (iNO) and electric NO generators (eNO). Nitric oxide gas was inhaled at 160 ppm in air for 15 minutes twice a day, before and after each work shift, over 14 days to healthcare providers. During NO administration vital signs were continuously monitored. Safety was assessed by measuring transcutaneous methemoglobinemia (SpMet) and the inhaled nitrogen dioxide (NO₂) concentration. 12 healthy healthcare professionals received 185 administrations of high dose NO (160 ppm) for 15 minutes twice daily. 171 doses were delivered by iNO and 14 doses by eNO. During NO administration SpMet increased similarly in both groups (p=0.82). Methemoglobin decreased in all subjects at five minutes after discontinuing NO administration. Inhaled NO₂ concentrations remained between 0.70 [0.63-0.79] and 0.75 [0.67-0.83] ppm in the iNO group and between 0.74 [0.68-0.78] and 0.88 [0.70-0.93] ppm in eNO group. During NO administration peripheral oxygen saturation and heart rate did not change. No adverse events occurred. Treatment with high dose inhaled NO (160 ppm) for 15 minutes twice a day using eNO is feasible and similarly safe when compared with iNO.

Nitric Oxide (NO) gas is approved by the United States Food and Drug Administration for the treatment of hypoxia associated with pulmonary hypertension in the newborn. In clinical practice, NO gas is widely used to reduce pulmonary artery pressure and to improve oxygenation in adult patients with acute respiratory distress syndrome (ARDS).

Nitric oxide gas is commonly administered with delivery systems that use, pressurized NO in nitrogen (NO/N₂) cylinders. Pressurized inhaled nitric oxide cylinders (denoted iNO) are widely available and have been used in more than half million patients worldwide. Despite being safe and reliable, the use of pressurized cylinder as the source of NO requires an extensive supply chain and trained personnel to deliver and manage the (NO/N₂) cylinders. Further, cylinder NO therapy can be expensive.

Electrical NO generators (eNO) have been proposed as an alternative source. These devices ionize air (nitrogen and oxygen) with a pulsed, high voltage electrical discharge leading to the generation of NO, nitrogen dioxide (NO₂) and metal micro particles (released by the electrodes during electrical discharge). A scavenger containing calcium hydroxide can reduce nitrogen dioxide (NO₂) levels below the safety threshold (less than 3 ppm for NO₂) while a high-efficiency 0.22 micron particulate air (HEPA) filter removes metal particles generated by electric discharge. These eNO devices can provide inhaled NO therapy without the need for bulky and expensive cylinders potentially making NO therapy widely available both inside and outside the hospital.

In an effort to evaluate the preventative effects of NO in COVID-19, a randomized clinical trial of healthcare workers was performed, with subjects receiving either iNO or eNO. This analysis aimed to evaluate the feasibility and safety of administering 160 ppm to spontaneously breathing healthy volunteers using iNO and eNO.

This analysis uses data from the randomized controlled trial of healthcare workers who were enrolled between March 2020 and August 2020. This study was reviewed and approved by the Institutional Review Board at Massachusetts General Hospital. Written informed consent was obtained from each subject prior to initiation of any study procedures. The trial was terminated early (Mar. 10, 2021) due a lack of enrollment after the approval of COVID-19 vaccines. Data from the enrolled participants who received nitric oxide were assessed for safety and feasibility of administration.

Enrolled subjects were adult (18 years) healthcare workers (physicians, nurses or respiratory therapists) working at Massachusetts General Hospital who were scheduled to work with SARS-CoV-2 positive patients at least three times a week (6 or more shifts in 14 days). Subjects were excluded if they previously had a positive SARS-CoV-2 reverse transcription polymerase chain reaction (RT-PCR) test, were pregnant or had a history of hemoglobinopathies or anemia.

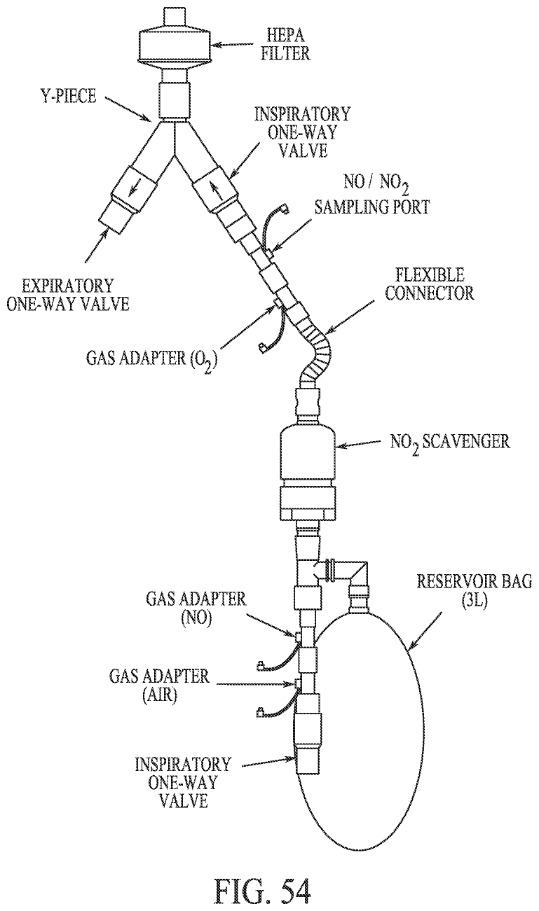

The study subjects received inhaled NO at 160 ppm for 15 minutes twice per day, before and after each work shift, over 14 days. To allow high concentrations of NO breathing, a facemask and apparatus that was previously designed and tested was utilized. The apparatus is composed of standard respiratory circuit connectors, a 3 L reservoir bag, a scavenger containing powdered calcium hydroxide, a 0.22 micron high-efficiency particulate air filter and a snug-fitting mask (see FIG. 54). Since high dose inhaled NO reacts with the circulating hemoglobin producing methemoglobin, transcutaneous methemoglobin (Masimo rainbow SET, Irvine, Calif. 92618) was monitored. Using the same device, peripheral oxygen saturation (SpO₂) and heart rate (HR) was evaluated. HR, SpO₂ and methemoglobinemia (SpMet) were collected before and at the end of NO administration. To continuously monitor the inspired fraction of oxygen (FiO₂) an oxygen analyzer (MiniOX® 1, Ohio Medical Corporation®, Gurnee, Ill. 60031) was used.

To avoid variation of NO gas concentration during the respiratory cycle, the reservoir gas flow was kept constantly at 15 L/min during NO treatments. Levels of NO and NO₂ were monitored through a sampling line connected to the inspiratory limb of the circuit. Inhaled NO was measured by chemiluminescence (Sievers 280i Nitric Oxide Analyzer, GE Analytical Instruments, Boulder Colo.) and Cavity Attenuated Phase Shift (CAPS) was used to monitor NO₂ levels (Aerodyne Research Inc, Billerica, Mass.). Additionally, NO-NO₂ tables were defined at 15 L/min air flow (Supplementary Table 13). To determine NO absorption and NO₂ production in the airway, exhaled NO and NO₂ concentrations were measured in one healthy subject during eNO administration.

TABLE 13
a table used to set nitric oxide (from a pressurized cylinder containing
850 ppm NO/N₂), air and oxygen flow necessary to reach
the desired concentration of 160 ppm.
NO = 160 ± 10 ppm
NO: Nitric oxide; O₂: oxygen; N₂; nitrogen.

| FiO₂ (%) | NO flow (L/min) | Air flow (L/min) | O₂ flow (L/min) |
|---|---|---|---|
| 21 | 4 | 15 | 1 |

Two different NO sources were studied, including a pressurized cylinder containing 850 ppm of NO/N₂ (150 A, Airgas, Radnor Township, Pa., content=4089 L at STP) and an electric NO generator. The electric NO generator (Portable NO generator, ODIC Inc. Littleton, Mass.) combines a gas pump, an NO generation chamber containing an iridium spark plug, an 18 g scavenger containing calcium hydroxide and a 0.22 micro meter HEPA filter. To obtain the desired NO concentration the generator was set with the following sparking parameters: air flow 2.5 L/min, sparking frequency 85 Hz and duty cycle 65%. The decision to use one source or another was due to material or personnel availability. Each subject can receive NO using both the sources ONO and eNO)

Data are reported as mean (standard deviation [SD]) or median (interquartile range [IQR]) for continuous variables and as frequencies and proportions for categorical variables. Normality was assessed using the Shapiro-Wilk test. To evaluate the trend of a continuous variable over time (before and after the treatment), a mixed effect model (R package [lme4] counting each patient as a random effect, R package [emmeans] for post hoc analysis) was used. Statistical significance was considered at a two-tailed P<0.05. All the analyses were conducted using R Core Team (2021).

NO was administered to 12 subjects, including six males and six females. Overall, the mean (SD) age was 43.3 (12.7) years with a body mass index of 28.9 (5.57) kg/m². Two subjects had a past medical history of systemic hypertension and diabetes mellitus, and one received chronic bronchodilator therapy for asthma.

Twelve subjects received a total of 185 NO gas administrations. iNO was used to administer 171 doses, and eNO was used for 14 doses. Each subject received, on average 15.4 NO administrations. All the study subject received NO using the iNO source. Three subjects received NO using both iNO and eNO. Air flow was maintained at 15 L/min in the reservoir for all treatments. An NO flow of 4 [0] L/min at 850 ppm in nitrogen was added when using pressurized cylinders, and 2.5 [0] L/min at 1180 ppm in air when using the electric NO generator. When an NO/N₂ pressurized cylinder was used, 1 L/min of supplemental oxygen was added to maintain the FiO₂ at 0.21.

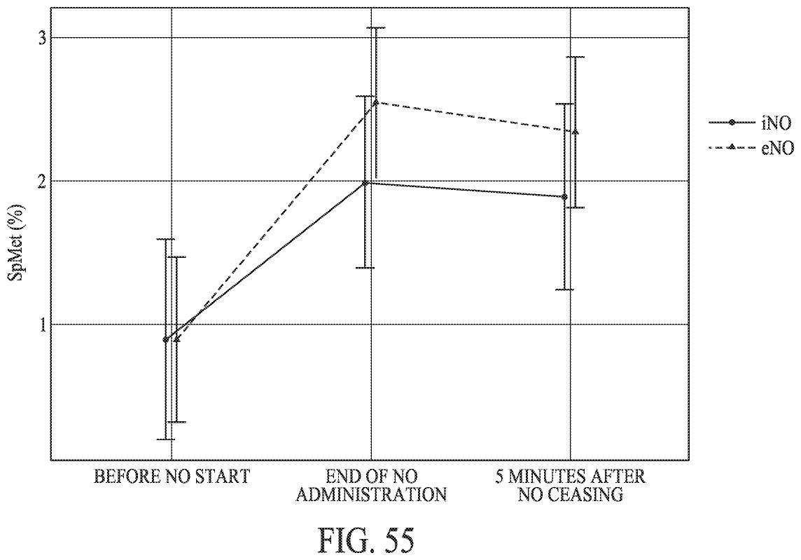

During the study gas administration SpMet increased in both groups from 0.90% (0.10) to 1.98% (0.11) with iNO (95% CI [−1.44; −0.70], P<0.001) and from 0.85% (0.17) to 1.89% (0.16) with eNO (95% CI [−1.64; −0.42], P<0.001). The increase of SpMet with eNO and iNO was not statistically different (95% CI [−0.29; 0.46], P=0.98; see FIG. 55). Five minutes after ceasing NO administration SpMet was decreased to 1.87% (0.11) (95% CI [−0.01; 0.22], P<0.098) and 1.81% (0.16) (95% CI [−0.32; 0.48], P=0.94) with iNO and eNO, respectively.

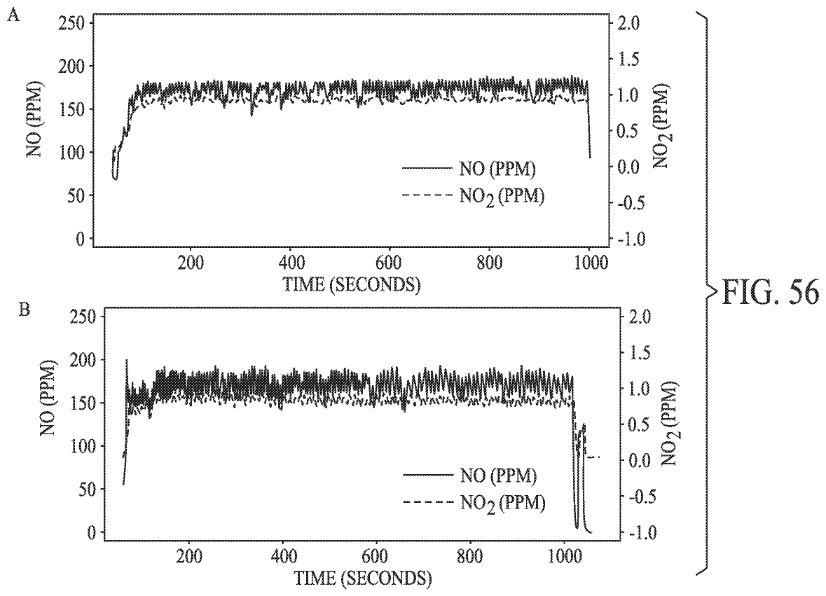

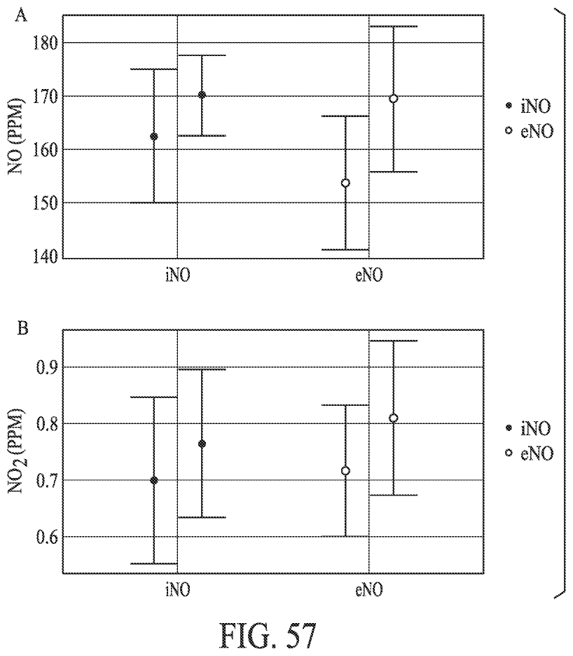

Inhaled NO and NO₂ concentrations were continuously monitored over 54 administrations, including 39 with iNO and 14 with eNO (see FIG. 56). During the study inhaled NO concentration median ranged between 164 [156-169] and 170 [165-175] ppm with iNO and between 153 [151-163] and 178 [158-180] ppm with eNO. NO₂ concentrations varied between 0.7 [0.63-0.79] and 0.75 [0.67-0.83] ppm with iNO and between 0.74 [0.68-0.78] and 0.88 [0.70-0.93] ppm with eNO (see FIG. 57). As shown, the intra-tidal variations of NO concentration are significantly higher with eNO 14.85 (10.60) ppm compared to iNO 8.53 (2.90) ppm (95% CI [−12.30; −0.35], P<0.38). This difference may be explained by the higher total fresh gas flow (air flow+NO flow+oxygen flow) with the iNO source 20 L/min as compared to eNO 17.5 L/min.

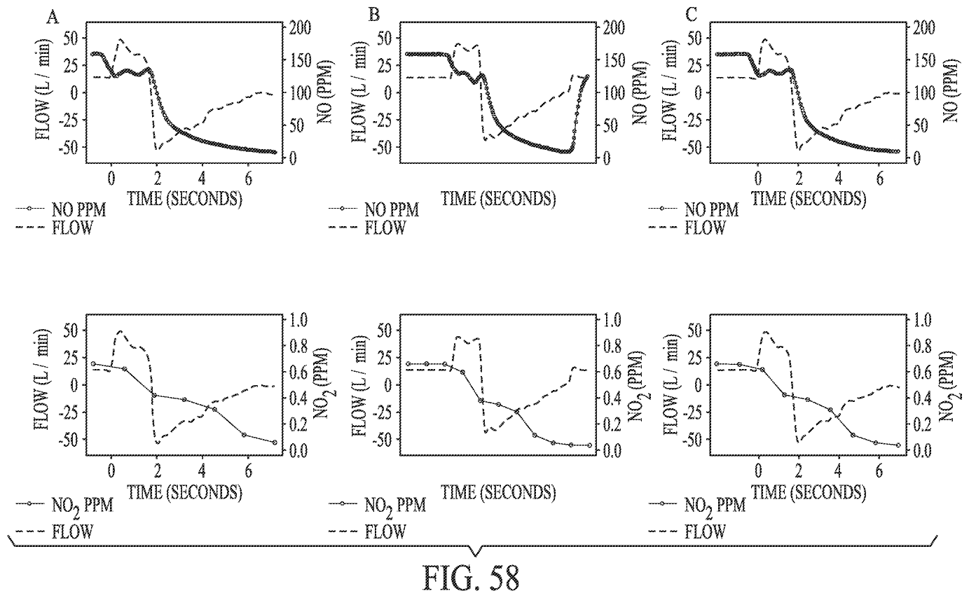

In one patient on eNO, the average inspired NO and NO₂ concentrations were 158.5 ppm and 0.68 ppm, respectively. At the end of exhalation (alveolar gas phase), NO concentration decreased to 11.8 ppm and NO₂ concentration was 0.27 ppm (see FIG. 58) suggesting that NO is bound to hemoglobin in the lungs with minimal NO₂ generation in airways.

SpO₂ decreased from 97% [97-98], before NO administration, to 96% [95-97] at the end of NO administration (95% CI [1.52; 2], P<0.001) with iNO. When eNO was used, SpO₂ remained unchanged (P=0.57). The observed 1-2% reduction of SpO₂ with iNO is most likely NO/N2 dilution of air.

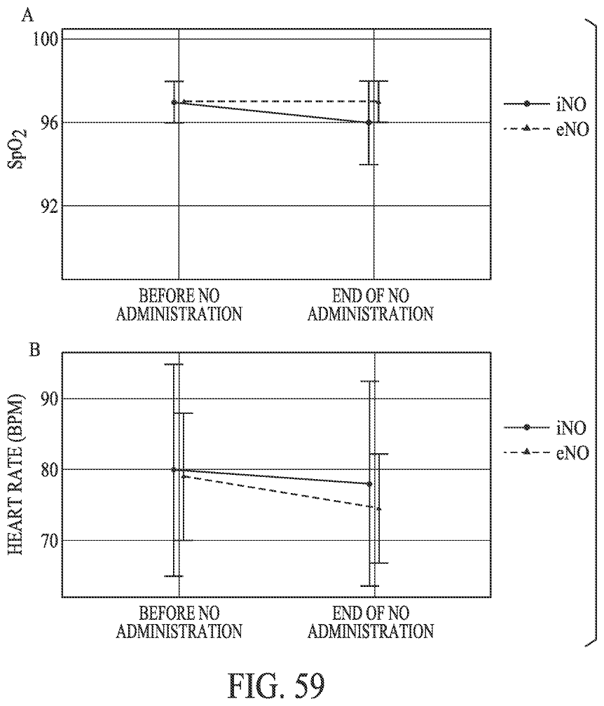

Heart rate was slightly reduced from 80 [72-87] beats-per minute (bpm) to 78 [70.5-85] bpm (95% CI [2; 3.5], P<0.001) with iNO and from 79 [73-82] bpm to 74.5 [70-77] bpm (95% CI [2.5; 8], P<0.001) with eNO. (see FIG. 59) During the administrations none of the study subjects reported any discomfort. No adverse events were noted.

Over 185 consecutive NO administrations, it was demonstrated that administering high dose NO (160 ppm) for 15 minutes using an electric NO generator is feasible and as safe compared to NO delivered from pressurized cylinder-based delivery systems. A stable NO concentration of 160 ppm was reached and maintained through the 15 minutes administration with both iNO and eNO. All NO administrations were well tolerated and without any adverse events. Volunteers were comfortable as demonstrated by their significant reductions of heart rate during the administrations. During the NO administrations SpMet rose in a similar fashion (with a percentage increase of 55%-64%) with both the NO sources suggesting a similar biological effect. Five minutes after the end of the administration SpMet decreased in both groups showing an adequate reduction of methemoglobin by the subjects' methemoglobin reductase. When administering NO at high levels, NO₂ concentration monitoring becomes imperative. Inhaled NO₂ concentration, despite being slightly higher with eNO, was below the Occupational Safety and Health Administration (OSHA) safety levels.

During the COVID-19 pandemic, six pregnant women were treated with severe or critical COVID-19 lung disease with high dose iNO (160-200 ppm) for 30 mins twice a day improving oxygenation and reducing respiratory rate. iNO produced symptomatic relief of shortness of breath and dyspnea in those patients. The main limitation of the widespread use of inhaled high dose NO is the need for dedicated personnel to manage bulky equipment and cylinders to patients. The electric NO generator that was used weighs 1.5 kg making its use easy when the availability of trained personnel or materials is lacking during the peak incidence of the ongoing COVID-19 pandemic or in low resource settings. The development of novel electric NO generators delivering high dose NO from air and that continuously monitor inhaled NO/NO₂ concentration and transcutaneous methemoglobin will make the use of eNO available for ambulatory and home use (particularly important for remote or low resource areas). The main limitation of this study is that the decision to use iNO or eNO was not due to a random allocation but depends on equipment availability consequently the two groups ONO and eNO) were very numerically unbalanced.

In this study it is demonstrated that it is feasible to administer high dose NO (160 ppm) using an electric NO generator. The increase in SpMet during NO gas delivery is comparable between iNO and eNO. Electric NO generation from air offers portability, usability and economy that may overcome the current limitations of cylinder-based NO delivery systems. The standard nitric oxide (NO) source is a pressurized cylinder containing nitric oxide balanced in nitrogen. In the last several years electric NO generators capable to generate NO from air using a pulsed electrical discharge have been developed. High dose NO was successfully administered to healthy volunteers using both pressurized cylinders and electric NO generators as a NO source. The delivery of high dose NO was feasible using both the sources. Methemoglobin increased in the same fashion in both groups. The delivered nitrogen dioxide levels were always below the prescribed safety levels.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the disclosure.

The use of the term “about” herein is defined as being plus or minus about 5% of the following value.

Various features and advantages of the disclosure are set forth in the following claims.

1. A nitric oxide generator, comprising:
an inlet arranged to receive a gas including nitrogen and oxygen;
an outlet;
a pair of electrodes arranged downstream of the inlet and configured to generate nitric oxide from the gas;
a pressure regulator configured to selectively adjust a pressure of the gas surrounding the electrodes;
an accumulator in communication with the pressure regulator, wherein the accumulator is configured to add volume to a flow path between the inlet and the outlet to store and maintain the pressure of the gas surrounding the electrodes;
a nitric oxide sensor arranged to measure a concentration of nitric oxide downstream of the electrodes; and
a controller in communication with the pair of electrodes, the pressure regulator, and the nitric oxide sensor, wherein the controller is configured to selectively instruct the pressure regulator to adjust the pressure of the gas surrounding the electrodes in response to the concentration of nitric oxide measured at the outlet by the nitric oxide sensor.

2. The nitric oxide generator of claim 1, further comprising a scavenger arranged upstream of the outlet and downstream of the electrodes.

3. The nitric oxide generator of claim 1, further comprising a filter arranged upstream of the outlet and downstream of the electrodes.

4. The nitric oxide generator of claim 1, wherein the controller is configured to instruct the pressure regulator to increase the pressure of the gas surrounding the electrodes to increase the concentration of nitric oxide at the outlet.

5. The nitric oxide generator of claim 1, wherein the controller is configured to instruct the pressure regulator to decrease the pressure of the gas surrounding the electrodes to decrease the concentration of nitric oxide at the outlet.

6. The nitric oxide generator of claim 1, wherein the controller is configured to selectively adjust a signal sent to the electrodes to provide an inhaled concentration of nitric oxide greater than or equal to about 150 ppm at the outlet.

7. The nitric oxide generator of claim 1, further comprising a methemoglobin sensor adapted to sense a methemoglobin level in a patient.

8. The nitric oxide generator of claim 7, wherein the controller is configured to monitor the methemoglobin level and instruct the pair of electrodes to decrease nitric oxide generation when the methemoglobin level reaches a predetermined threshold value.

9. A nitric oxide generator, comprising:
an inlet arranged to receive a gas including nitrogen and oxygen;
an outlet;
a pair of electrodes arranged downstream of the inlet and configured to generate nitric oxide from the gas;
a nitric oxide sensor arranged to measure a concentration of nitric oxide downstream of the electrodes; and
a controller in communication with the pair of electrodes and the nitric oxide sensor, wherein the controller is configured to selectively adjust a signal sent to the electrodes to provide an inhaled concentration of nitric oxide greater than or equal to about 150 ppm at the outlet.

10. The nitric oxide generator of claim 9, further comprising an accumulator in communication with the pressure regulator.

11. The nitric oxide generator of claim 10, wherein the accumulator is configured to add volume to a flow path between the inlet and the outlet to store and maintain the pressure of the gas surrounding the electrodes.

12. The nitric oxide generator of claim 9, further comprising a pressure regulator configured to selectively adjust a pressure of the gas surrounding the electrodes.

13. The nitric oxide generator of claim 12, wherein the controller is in communication with the pressure regulator and the controller is configured to selectively instruct the pressure regulator to adjust the pressure of the gas surrounding the electrodes in response to the concentration of nitric oxide measured at the nitric oxide sensor.

14. The nitric oxide generator of claim 13, wherein the controller is configured to instruct the pressure regulator to increase the pressure of the gas surrounding the electrodes to increase the concentration of nitric oxide at the outlet.

15. The nitric oxide generator of claim 13, wherein the controller is configured to instruct the pressure regulator to decrease the pressure of the gas surrounding the electrodes to decrease the concentration of nitric oxide at the outlet.

16. The nitric oxide generator of claim 9, wherein further comprising a methemoglobin sensor adapted to sense a methemoglobin level in a patient.

17. The nitric oxide generator of claim 9, wherein the controller is configured to monitor the methemoglobin level and instruct the pair of electrodes to decrease nitric oxide generation when the methemoglobin level reaches a predetermined threshold value.

18. The nitric oxide generator of claim 9, further comprising a scavenger arranged upstream of the outlet and downstream of the electrodes.

19. The nitric oxide generator of claim 9, further comprising a filter arranged upstream of the outlet and downstream of the scavenger.

20. A nitric oxide delivery system, comprising:
an inlet;
a first outlet;
a main conduit connected between the inlet and the first outlet;
a nitric oxide generator including a pair of electrodes configured to generate a predetermined inhaled concentration of nitric oxide;
a reservoir connected to the main conduit downstream of the nitric oxide gas source, wherein the reservoir is configured to add an amount of volume a flow path defined between the inlet and the first outlet;
a scavenger arranged between the reservoir and the first outlet;
a filter arranged between the reservoir and the first outlet;
an inspiratory valve arranged downstream of the reservoir and configured to allow gas flow only in a direction from the reservoir toward the first outlet;
an expiratory valve arranged downstream of the first outlet and configured to allow gas flow only in a direction from the first outlet to a second outlet.

21-68. (canceled)
